(19) United States
(12) Patent Application Publication
(10) Pub. No.: US 2026/0069711 A1
(43) Pub. Date: Mar. 12, 2026

(54) SYSTEMS, DEVICES AND METHODS FOR MODULAR FUNCTIONALIZED CELLULAR NANOSTRUCTURES FOR TARGETED PAYLOAD DELIVERY

(71) Applicant: THE REGENTS OF THE UNIVERSITY OF CALIFORNIA, Oakland, CA (US)

(72) Inventors: Liangfang Zhang, San Diego, CA (US); Ronnie H. Fang, San Diego, CA (US); Weiwei Gao, La Jolla, CA (US); Nishta Krishnan, La Jolla, CA (US); Yao Jiang, La Jolla, CA (US)

(21) Appl. No.: 18/830,468

(22) Filed: Sep. 10, 2024

(51) Int. Cl. A61K 47/69; A61K 45/06; A61K 47/64; B82Y 5/00

(52) U.S. Cl. CPC A61K 47/6929; A61K 45/06; A61K 47/64; A61K 47/6901; B82Y 5/00

(57) ABSTRACT

Disclosed are devices, systems, and methods of manufacture and use of modular functionalized cellular nanoparticles that can bind a wide range of ligands, payloads, and functional substances onto a nanoparticle surface. In various embodiments, a cell membrane coating on the nanoparticle is engineered to express a membrane anchor that can readily form a covalent bond with any functional moiety modified with an appropriate peptide tag sequence.

### TECHNICAL FIELD

This patent document relates to devices, systems, and methods that use nanoscale material technologies.

### BACKGROUND

Nanotechnology provides techniques or processes for fabricating structures, devices, and systems with features at a molecular or atomic scale, e.g., structures in a range of one to hundreds of nanometers in some applications. For example, nano-scale devices can be configured to sizes similar to some large molecules, e.g., biomolecules such as enzymes. Nano-sized materials used to create a nanostructure, nanodevice, or a nanosystem can exhibit various unique properties, e.g., including optical, electrical, and/or mechanical properties, that are not present in the same materials at larger dimensions and such unique properties can be exploited for a wide range of applications.

### SUMMARY

Disclosed are devices, systems, and methods of manufacture and use of modular functionalized cellular nanoparticles (CNPs) that encompass a wide range of ligands onto the nanoparticle surface. In some embodiments, for example, a nanoparticle device includes a cell membrane coating on a nanoparticle (e.g., nanosphere, nanorod, nanodisc, etc.) that is engineered to express a surface-bound membrane anchor (e.g., protein) that can readily attach any functional soluble ligand modified with a tag sequence (e.g., peptide or protein tag) that conjugates with the surface-bound membrane anchor, where the functional soluble ligand is able to bind to specific receptors on a targeted cell, thereby modularizing the nanoparticle device to target any cell. In some embodiments, for example, a modular functionalized nanoparticle device includes a nanosphere having a coating derived from a cellular membrane that has been engineered to express SpyCatcher as the surface-bound membrane anchor proteins, which provides a base for conjugating with an array of SpyTag-modified soluble ligands selected to bind to receptor(s) of a target cell, thereby providing a modular, scalable, functionalized nanoparticle platform for a plethora of applications. In some embodiments, for example, a nanoparticle device includes a nanodisc structure having the engineered cell membrane coating expressing a plurality of SpyCatcher, anchor proteins conjugated with SpyTag-labeled soluble ligands.

In some aspects, a modular functionalized nanoparticle includes a nanoparticle core; a cell membrane coating on the nanoparticle core; and a molecular binding complex natively bound to the cell membrane coating, the molecular binding complex comprising an anchor compound integrated with the cell membrane coating, and a tag molecule having a specific binding affinity to the anchor compound and bound to the anchor compound, wherein the tag molecule is configured to couple a functional substance to the molecular binding complex so as to facilitate one or more of targeting, attachment, or payload delivery by the modular functionalized nanoparticle on a target cell.

In some aspects, a method for fabricating a modular functionalized nanoparticle includes forming a cell membrane coated nanoparticle, wherein the cell membrane coated nanoparticle comprises a nanoparticle core and a cell membrane coating on the nanoparticle core having an anchor compound natively bound to the cell membrane coating; forming a modular molecular linkage complex, wherein the modular molecular linkage complex comprises (i) a tag molecule having a specific binding affinity to the anchor compound, and (ii) a functional substance coupled to the tag molecule; and producing the modular functionalized nanoparticle by functionalizing the cell membrane coated nanoparticle with the modular molecular linkage complex.

The subject matter described in this patent document can be implemented in specific ways that provide one or more of the following features.

### BRIEF DESCRIPTION OF THE DRAWINGS

FIG. 1A shows a diagram of an example embodiment of a modular functionalized nanoparticle, in accordance with the disclosed technology.

FIG. 1B shows a diagram illustrating exemplary genetically engineered modular functionalized cellular nanoparticles (CNPs) and an example embodiment of a method for fabricating and functionalizing the cellular membrane nanoparticles via SpyCatcher-expressing cells and SpyTag-labeled ligands, in accordance with the disclosed technology.

FIGS. 1C-1I show example characterization data of the example implementations of the exemplary processes and of the modular functionalized CNPs depicted in FIG. 1A.

FIGS. 2A-2K show data plots and fluorescent images depicting functional characterization of SpyTag-labeled ligands from example implementations of the disclosed technology.

FIG. 3 shows a diagram of an example embodiment of a method for fabricating a modular functionalized nanoparticle, in accordance with the present technology.

FIGS. 4A-4C show example embodiments of optional processes of the method for fabricating a modular functionalized nanoparticle shown in FIG. 3.

FIGS. 5-8 show data plots and images of example characterization results of SpyTag-labeled ligands and conjugation with SpyCatcher and targeted cell surface receptors from example implementations of the disclosed technology.

FIGS. 9A-9L show data plots and images from example implementations of nanoparticle synthesis and functionalization techniques in accordance with the disclosed technology.

FIGS. 10-15 show data plots and images of example characterization results of fabrication of modular, scalable CNPs, in accordance with the disclosed technology.

FIGS. 16-18D show data plots and images of example characterization results of cell membrane functionalization on modular, scalable CNPs, in accordance with the disclosed technology.

FIG. 19A-19L show data plots and images from example implementations demonstrating functional characterization of modularly functionalized nanoparticles, in accordance with the present technology.

FIGS. 20-27 show data plots and images of example in vitro characterization results of modular functionalization on cell membrane-coated CNPs and target cell binding, in accordance with the present technology.

FIG. 28A-28M show images and data plots from example implementations demonstrating in vivo tumor targeting, therapeutic efficacy, and safety of the exemplary modularly functionalized nanoparticles, in accordance with the present technology.

FIGS. 29-33 show images and data plots of example in vivo characterization results of modular functionalization on cell membrane-coated CNPs and target cell binding, in accordance with the present technology.

FIG. 34 shows a schematic representation of the example engineered constructs of the example implementations.

FIG. 35A shows a diagram depicting an example embodiment of a modular functionalized of cellular nanostructure for targeted tumor delivery, in accordance with the present technology.

FIG. 35B shows a diagram depicting an example embodiment of a method for modular functionalization of cellular nanodisc (CND) for targeted tumor delivery, in accordance with the present technology.

FIGS. 36A-36H show data images and data plots depicting an example fabrication implementation of example embodiments of the CNDs and characterization of the fabricated CNDs.

FIGS. 37A-37E show data images and data plots depicting an example in vitro characterization of example embodiments of the CNDs.

FIGS. 38A-38D show data plots and data images depicting an example in vivo characterization of example embodiments of the CNDs.

FIGS. 39A-39E show data plots depicting example results of example implementations to evaluate therapeutic efficacy and in vivo safety of example embodiments of the CNDs.

### DETAILED DESCRIPTION

In recent years, cellular nanoparticles (CNPs) have shown significant promise for the diagnosis, treatment, and prevention of disease. By coating synthetic nanoparticulate cores with cell membranes, the resulting CNPs display an array of surface markers that enable them to recapitulate natural cellular interactions and be used for a range of biomedical applications.

For example, red blood cell nanoparticles inherit “markers of self” that promote immune evasion, resulting in significantly improved circulation times. Likewise, platforms based on platelets, white blood cells, cancer cells, and bacteria, among others, possess unique properties that have allowed them to excel in various situations. The functionality of CNPs can been explored through lipid insertion, chemical conjugation, metabolic engineering, and genetic engineering. Through modifications like these, it is possible to design next-generation CNP platforms with altered biointerfacing capabilities. For instance, genetic editing has been a robust and flexible approach for expressing engineered ligand proteins on the cellular membrane, which are then used to coat nanoparticles and provide augmented function, including for immune modulation, disease targeting, and endosomal escape.

While manipulating the surface expression of membrane-bound proteins can be reasonably straightforward, anchoring soluble proteins or other target payloads onto the cell membrane without abolishing the native structure-function is substantially challenging. The use of soluble protein ligands greatly increases the number of intra-and inter-cellular targets that can be bound for innumerable applications, yet this functionality is limited by the technical challenges of fusing and anchoring a soluble protein ligand (not natively membrane-bound) onto a given substrate. This can be achieved in some instances by anchoring the soluble protein ligand onto the cell surface by fusing it with the transmembrane domain of another membrane-bound protein. However, these resulting fusion proteins must be optimized on a case-by-case basis and are frequently prone to misfolding, reduced function due to steric hindrance, and low expression levels. For example, to produce a genetically-engineered cellular nanoparticle for a particular biomedical application, it can typically involve several months (e.g., three months on average) to devise, prepare and produce, and validate the genetically-modified cellular nanoparticle for the particular application. As such, engineering a single membrane-anchored soluble protein ligand for expression on a cell surface through conventional means entails a prohibitive amount of financial cost and time, limiting the ability to create a functionalized cell-membrane-based coating for a CNP in any given application and thus thwarts the advancement and usefulness of CNPs for the plethora of biomedical applications to which they could be applied.

There have been new strategies to decouple the expression of the transmembrane surface-bound anchor and the soluble ligand or payload in attempts to provide modular approaches for functionalization. Avidin-biotin binding and click chemistry have been used to link soluble proteins or other targeted payload molecules to nanoparticle surfaces. However, these applications face several limitations. For instance, click chemistry reactions can be challenging in live-cell applications due to the generally higher cost of bio-compatible reagents. Additionally, incorporating non-canonical, click-chemistry-functionalized amino acids into proteins requires expensive and labor-intensive genetic engineering. Often, low expression levels of these non-canonical amino acids within the expressed proteins further complicate the process. Similarly, avidin-biotin binding applications are limited by issues such as biotin tags causing aggregation and changes in the solubility of engineered proteins, time-and energy-intensive post-processing of the expressed proteins, and the high cost of recombinant biotin ligase enzyme, which can inhibit scalable production. Therefore, these have not been shown to be effective modular functionalization strategies that could enable the development of nanomedicine platforms against viruses, bacteria, malarial infections, and cancers. What is needed is a scalable, on-demand functionalized cell membrane-coated nanoparticle system which can be modularly adapted for addressing the needs across diverse biomedical applications.

Disclosed are devices, systems, and methods of manufacture and use of modular functionalized CNPs that are able to incorporate a wide range of soluble ligands or functionalized substances onto the nanoparticle surface. In various embodiments, a cell membrane coating on the nanoparticle is engineered to express a membrane-bound anchor (e.g., anchor protein) that can readily attach any substance, such as a target ligand, payload, or any functional moiety, that is modified with a tag (e.g., peptide or protein tag) that correspondingly binds to the membrane-bound anchor. In some embodiments, a modular functionalized nanoparticle in accordance with the present technology includes a nanoparticle core, a cell membrane coating on the nanoparticle core, and a molecular binding complex natively bound to the cell membrane coating, where the molecular binding complex includes an anchor compound (e.g., anchor protein) integrated with a lipid bilayer of the cell membrane coating, and a tag molecule (e.g., peptide tag or protein tag) that has a binding affinity to the anchor compound, and where the tag molecule of the molecular binding complex is able to bind a substance (e.g., a target ligand having a binding affinity to a receptor of a target cell, or a payload substance to deliver for uptake by a target cell), so as to facilitate targeting, attachment, and/or other function by the modular functionalized nanoparticle on a target cell. In some embodiments, for example, the molecular binding complex is formed through an iso-peptide covalent bond between the anchor compound and the tag molecule; whereas, in some embodiments, for example, the molecular binding complex is formed through a coiled coil peptide interaction. In some embodiments, for example, the molecular binding complex includes a catcher/tag protein-ligand binding system; whereas in some embodiments, for example, the molecular binding complex includes a leucine zipper binding system.

Some example embodiments of the disclosed devices, systems, and methods for a modular functionalized CNP platform include a genetically engineered cell membrane expressing SpyCatcher or other Catcher variants as the membrane-bound anchor protein conjugated with a SpyTag-modified soluble ligand or other Tag-modified ligands selected to bind to a receptor of a target cell, thereby providing a modular, scalable, functionalized nanoparticle platform for a variety of biomedical applications. Overall, all variants of the Tag/Catcher ligand-protein binding system provide an irreversible conjugation of recombinant proteins involving a peptide (referred to as a “Tag”) that reacts with a larger protein (referred to as a “Catcher”) to form an intermolecular covalent isopeptide bond between the pair with high specificity. The reactions involving these Tag/Catcher pairs proceed under mild, biologically-friendly reaction conditions and are fully self-catalyzed.

For example, in some embodiments, after coating the nanoparticle core with the genetically-engineered modified membrane expressing SpyCatcher or any similar Catcher protein, the resulting base nanoformulation can be functionalized with an array of complementary Tag-labeled ligands. The genetically-engineered cells expressing SpyCatcher or any similar Catcher proteins are able to be produced in large amounts relatively quickly because the stable cell line does not require modification for different desired applications or targets and the Catcher proteins do not require extensive post-processing steps after the cell membrane is coated onto the nanoparticle. The exemplary SpyTag/SpyCatcher ligand-protein binding system (also referred to as “SpyCatcher system”) provides an irreversible conjugation of recombinant proteins involving a 13 amino acid peptide (referred to as “SpyTag”) that reacts with a 12.3 kDa protein (referred to as “SpyCatcher”) to form an intermolecular covalent isopeptide bond between the pair. The SpyTag-SpyCatcher binding pair may be used in the present technology for conjugating proteins onto nanoparticle surfaces. Using this system, a wide range of soluble ligands can be fused with the SpyTag oligopeptide, enabling them to be covalently attached to a SpyCatcher-functionalized substrate through the spontaneous formation of an isopeptide bond. The soluble ligands fused with the SpyTag oligopeptide are able to be produced in large amounts relatively quickly because these recombinant soluble ligands with SpyTag do not require extensive post-processing steps after extraction and purification to confer functionality. For example, in the case of recombinant production of the SpyTag-modified ligands, there is no requirement to purify out unreacted components or to remove harmful solvents.

Some example embodiments of the disclosed devices, systems, and methods for a modular functionalized CNP platform, in accordance with the present technology, include a genetically engineered cell membrane expressing SnoopCatcher as the membrane-bound anchor protein conjugated with a SnoopTag-modified soluble ligand selected to bind to a receptor of a target cell, thereby providing a modular, scalable, functionalized nanoparticle platform for a variety of biomedical applications. The exemplary embodiments of the SnoopCatcher/SnoopTag-type modular functionalized CNP platform is operable to function similar to and in parallel with the exemplary embodiments of the SpyCatcher/SpyTag-type modular functionalized CNP platform disclosed herein, for example, as the SnoopTag/SnoopCatcher is a fully orthogonal ligand/protein pair developed from the RrgA protein of Streptococcus pneumoniae that has no cross-reactivity with SpyTag/SpyCatcher system and forms an isopeptide bond between a Lys-Asn instead of Lys-Asp found in SpyTag/SpyCatcher system. Also, for example, the SnoopCatcher/SnoopTag-type modular functionalized cellular nanoparticles are able to be fabricated in a manner similar to the exemplary SpyCatcher/SpyTag-type modular functionalized CNP platform, where, for example, after coating the nanoparticle core with the genetically-engineered modified membrane expressing SnoopCatcher, the resulting base nanoformulation can be functionalized with an array of SnoopTag-labeled ligands.

Some example embodiments of the disclosed devices, systems, and methods for a modular functionalized CNP platform, in accordance with the present technology, include a genetically engineered cell membrane expressing DogCatcher as the membrane-bound anchor protein conjugated with a DogTag-modified soluble ligand selected to bind to a receptor of a target cell, thereby providing a modular, scalable, functionalized nanoparticle platform for a variety of biomedical applications. The exemplary embodiments of the DogCatcher/DogTag-type modular functionalized CNP platform is operable to function similar to and in parallel with the exemplary embodiments of the SpyCatcher/SpyTag-type modular functionalized CNP platform disclosed herein. For example, the DogCatcher/DogTag protein-ligand system is derived from the same domain from RrgA as the SnoopCatcher/SnoopTag protein-ligand system, but is engineered for optimal reaction kinetics by incorporating a proline-based loop, which reduces flexibility. Also, for example, the DogCatcher/DogTag-type modular functionalized cellular nanoparticles are able to be fabricated in a manner similar to the exemplary DogCatcher/DogTag-type modular functionalized CNP platform, where, for example, after coating the nanoparticle core with the genetically-engineered modified membrane expressing DogCatcher, the resulting base nanoformulation can be functionalized with an array of DogTag-labeled ligands.

Some example embodiments of the disclosed devices, systems, and methods for a modular functionalized CNP platform, in accordance with the present technology, include a genetically engineered cell membrane expressing a protein with a coiled-coil motif as the membrane-bound anchor protein conjugated with a coiled-coil modified soluble ligand selected to bind to a receptor of a target cell, thereby providing a modular, scalable, functionalized nanoparticle platform for a variety of biomedical applications. The exemplary embodiments of the coiled-coil-type modular functionalized CNP platform, which may comprise a leucine zipper coiled-coil motif, has similar advantages and may operate in parallel with the exemplary embodiments of the SpyCatcher/SpyTag-type modular functionalized CNP platform disclosed herein. For example, while the coiled-coil protein-ligand system is characterized by the alpha-helical dimers and stabilized by hydrophobic interactions, it shares the advantages of the Catcher/Tag system in that it is genetically encoded and forms molecular binding complex natively bound to the cell membrane coating, without the need of post-processing to confer functionality to the binding complex and wherein the molecular binding complex is capable of self-assembling. Also, the coiled-coil modular functionalized cellular nanoparticles are able to be fabricated in a manner similar to the exemplary Catcher/Tag-type modular functionalized CNP platform, where, after coating the nanoparticle core with the genetically-engineered modified membrane expressing coiled-coil anchor protein, the resulting base nanoformulation can be functionalized with an array of coiled-coil-labeled ligands without the need for additional enzymes, expensive reagents, or harsh solvents.

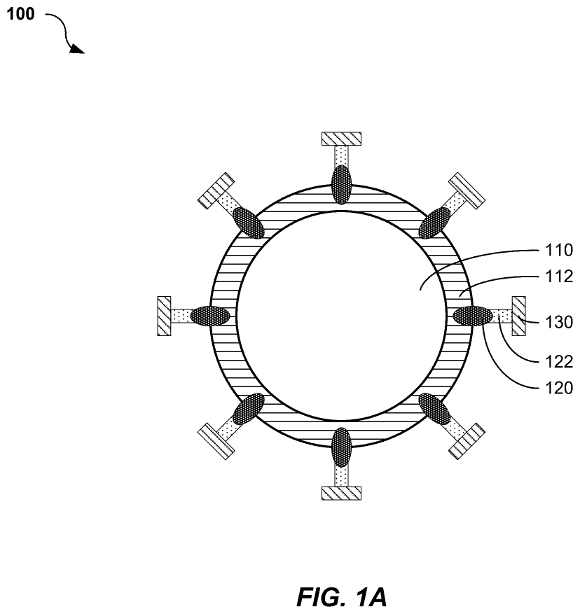

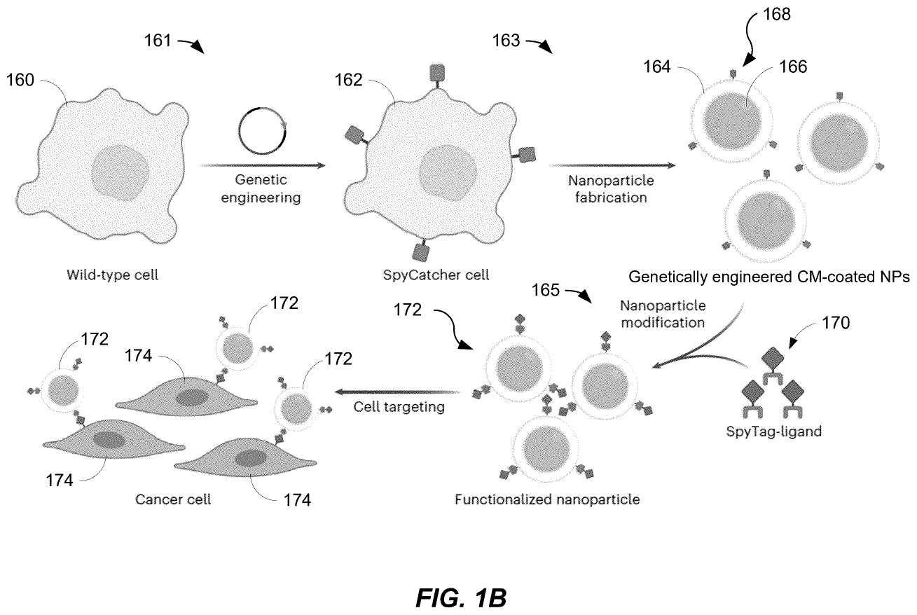

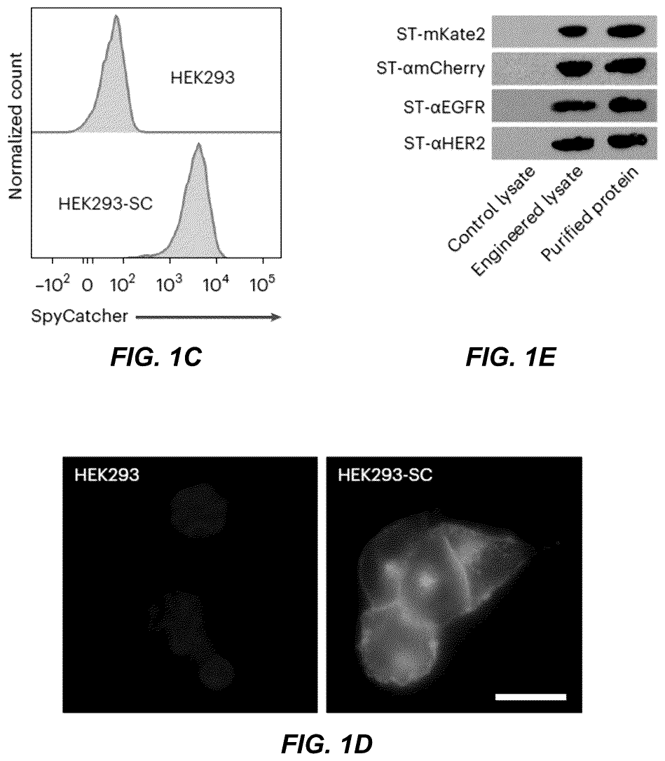

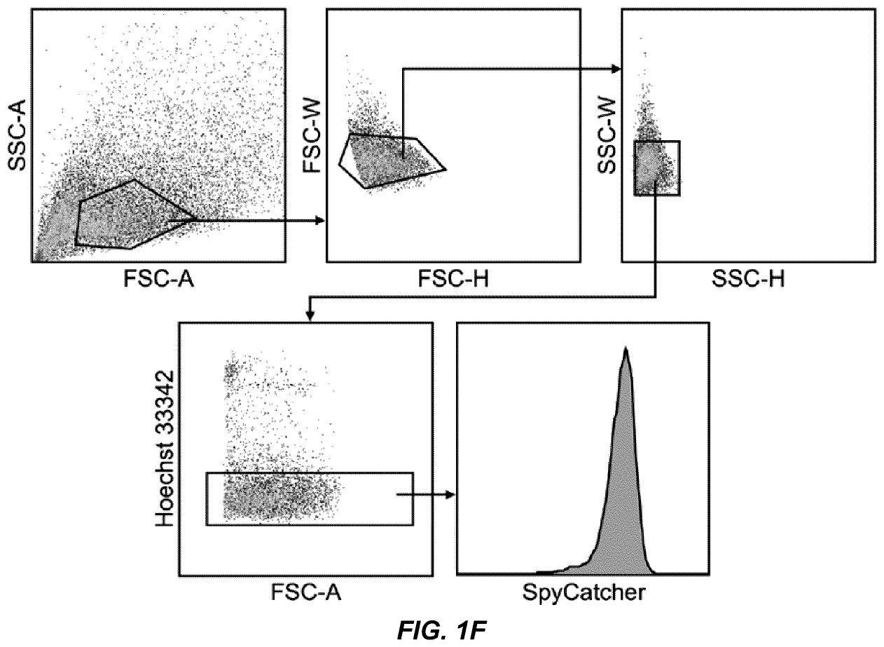

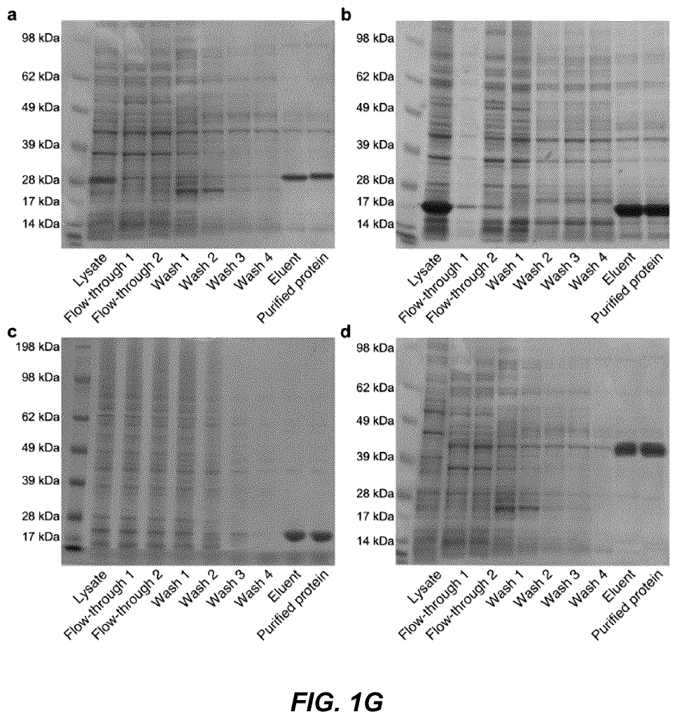

FIG. 1A shows a diagram of an example embodiment of a modular functionalized nanoparticle 100, in accordance with the present technology. The modular functionalized nanoparticle 100 includes a nanoparticle core 110. The modular functionalized nanoparticle 100 includes a cell membrane coating 112 on the nanoparticle core 110. The modular functionalized nanoparticle 100 includes a surface-bound anchor 120 (e.g., an anchor protein) that is natively bound to the cell membrane coating 112. The modular functionalized nanoparticle 100 includes a tag 122 (e.g., peptide tag or protein tag) having a binding affinity to a region of the surface-bound anchor 120 exposed from the cell membrane coating 112. For instance, the binding affinity between the tag 122 and the surface-bound anchor 120 (e.g., anchor protein) is exclusive, in which the tag 122 and surface-bound anchor 120 have a binding affinity just for each other that excludes off-target binding of other substances, i.e., no other moiety can bind to the surface-bound anchor 120. The modular functionalized nanoparticle 100 can include a target functionalization substance 130 coupled to the tag 122. In some embodiments, for example, the target functionalization substance 130 includes a ligand that has a binding affinity to a receptor of a target cell (not shown) for enabling attachment or uptake of the modular functionalized nanoparticle 100 to or by the target cell; whereas, in some embodiments, for example, the target functionalization substance 130 includes a payload (e.g., molecular payload) that is to be delivered to the target cell.

In some embodiments of the modular functionalized nanoparticle 100, for example, the nanoparticle core 110 includes a nanosphere, a nanodisc, a nanorod, or a nanocone. In some embodiments, for example, the nanoparticle core 110 includes a polymeric nanoparticle; an inorganic nanoparticle, such as a gold nanoparticle, silver nanoparticle, a copper nanoparticle, or other; a ceramic nanoparticle such as an iron oxide nanoparticle, or other oxide, carbide, carbonate, or phosphate based nanoparticle; a carbon nanoparticle comprising a carbon material including but not limited to diamond, carbon black, graphene, graphite, carbon nanotubes, or other; a lipid nanoparticle; a protein nanoparticle, including a protein material that includes but is not limited to collagen, keratin, elastin, or other; and/or a hydrogel nanoparticle. In some embodiments, for example, the nanoparticle core 110 is configured to have at least one dimension (e.g., diameter, length, height, etc.) in a range of 30 nm to 1 μm. For example, some embodiments of the exemplary nanosphere, nanodisc, nanorod, nanocone, or other type of nanoparticle core 110 can be configured to have a smallest dimension of about 30 nm to facilitate formation of the cell membrane coating 112 on the outer surface of the nanoparticle core 110, where the cell membrane coating 112 can have a thickness of about 10 nm. In some embodiments, for example, the nanoparticle core 110 is configured to have at least one dimension in a range of 80 nm to 120 nm, e.g., such as a diameter of 80 nm to 120 nm, or a length or height of 80 nm to 120 nm.

In some embodiments of the modular functionalized nanoparticle 100, for example, the surface-bound anchor 120 includes a catcher protein (e.g., a SpyCatcher protein, a SnoopCatcher protein, and/or a DogCatcher protein), and the tag 122 includes a corresponding or pairing peptide tag (e.g., a SpyTag peptide tag, a SnoopTag peptide tag, and/or a DogTag peptide tag, respectively). For example, the binding interaction between the surface-bound anchor 120 and the tag 122 can be covalent (e.g., in the case of tag/catcher ligand-protein system, such as SpyTag/SpyCatcher, SnoopTag/SnoopCatcher, and DogTag/DogCatcher) or based on physical interactions (e.g., such as hydrophobic interactions or hydrogen bonding). In some embodiments, for example, the tag 122 is bound to the surface-bound anchor 120 (e.g., anchor protein) through an iso-peptide covalent bond; whereas, in some embodiments, for example, the tag 122 is bound to the surface-bound anchor 120 (e.g., anchor protein) through a coiled coil peptide interaction.

In some embodiments of the modular functionalized nanoparticle 100, for example, the cell membrane coating 112 is derived from a cell membrane of a cell genetically-engineered to express the example anchor protein (i.e., example embodiment of the surface-bound anchor 120) such that it is bound to the cell membrane, which can be extracted from the cell to create the cell membrane coating 112 with the example anchor protein already bound (e.g., integrated in the lipid bilayer of the cell membrane of the cell membrane coating 112). In some embodiments of the modular functionalized nanoparticle 100, for example, the surface-bound anchor 120 (e.g., anchor protein) and the tag 122 (e.g., peptide or protein tag) together form a molecular binding complex is configured to readily attach any functional material such as a soluble ligand, a molecular payload, or other, i.e., any embodiment of the target functionalization substance 130. For example, the modular functionalized nanoparticle 100 is designed to be modular and versatile to virtually any application because the functionalization substance 130 can be configured with the tag 122 of the exemplary molecular binding complex, enabling the base cellular nanoparticle with the application-tailored tag of the molecular binding complex to be suitable for whatever application the functionalization substance 130 can be used for. For example, in some implementations, the functionalization substance 130 can simply be synthesized, expressed, or ligated to include the tag 122 as part of it or to chemically conjugate a binding sequence that corresponds to a tag sequence of the tag 122. Example synthesis, ligation or chemical conjugation methods to incorporate the tag sequence with a functionalization substance may comprise, but are not limited to, disulfide bonding, an amide bond, bio-orthogonal reactions, photo-crosslinking, or other technique. In some embodiments of the modular functionalized nanoparticle 100, for example, the tag 122 (e.g., peptide or protein tag) and the target functionalization substance 130 (e.g., ligand, payload, etc.) together form a modular linkage complex that is conjugated to the surface-bound anchor 120 (e.g., anchor protein).

In some embodiments of the modular functionalized nanoparticle 100, for example the modular functionalized nanoparticle 100 includes a payload, such as a molecular payload. For example, the nanoparticle 100 with the payload is able to be uptaken by the target cell based on the attachment or uptake of the modular functionalized nanoparticle 100 to or by the target cell, thereby providing targeted delivery of payload to the target cell by the modular functionalized nanoparticle 100. In some embodiments, for example, the molecular payload includes a drug, a prodrug, a nucleic acid substance, a protein substance, and/or a lipid substance. For example, an exemplary nucleic acid substance can include, but is not limited to, one or more of a nucleotide, an oligonucleotide, an oligonucleotide-based aptamer, a deoxyribonucleic acid (DNA) or portion thereof, or a ribonucleic acid (RNA) or portion thereof. For example, an exemplary protein substance can include, but is not limited to, one or more of an amino acid, a peptide, a peptide-based aptamer, an enzyme, an antibody, or a hormone. For example, an exemplary lipid substance can include, but is not limited to, one includes one or more of a liposome, a glyceride, a fatty acid, a steroid, or a phospholipid. In some embodiments, for example, the payload can be incorporated inside of the nanoparticle core 110. In such examples using a small molecule payload, the small molecule payload may diffuse out of the modular functionalized nanoparticle 100 once the modular functionalized nanoparticle 100 binds to the target cell or tissues. In some embodiments, for example, the payload can be attached to the surface of the cell-membrane coating 112 of the modular functionalized nanoparticle 100, e.g., in addition to the attachment of the surface-bound anchor 120 and the tag 122 (e.g., a molecular binding complex). For instance, the payload can be attached to the modular functionalized nanoparticle 100 by physical conjugation to the cell-membrane coating 112. Whereas, in some embodiments, for example, the payload can be attached to the modular functionalized nanoparticle 100 by using an orthogonal anchor/tag system like the surface-bound anchor 120/tag 122 molecular binding complex, discussed above. As an illustrative example, a biological drug payload, e.g., including protein payload and/or mRNA payload, can be delivered to the cytosol directly through an endosomal escape mechanism.

The disclosed modular functionalized cellular nanostructures can be implemented in a variety of ways to achieve technical advantages for various applications. For example, an exemplary CNP platform in accordance with the present technology can be readily modified with soluble ligands in a modular manner. Using the same base formulation, for example, a variety of components can be attached onto the nanoparticle surface to introduce new functionalities. Modular functionalized CNPs in accordance with the present technology exhibit exceptional binding to cancer cells expressing the corresponding cognate receptor both in vitro and in vivo.

### Example Embodiments and Implementations of Modular Functionalized Cellular Nanostructures Through Genetic Engineering

Some embodiments of the disclosed modular functionalized cell membrane-coated nanostructure technology include a modular CNP platform by genetically engineering the cell membrane to express SpyCatcher as an anchor. Also disclosed are methods for manufacturing the exemplary modular CNP functionalized with a SpyTag/SpyCatcher ligand-protein binding system. For example, in some embodiments, a fabrication method includes coating a modified membrane onto a nanoparticle core, and functionalizing the resulting base nanoformulation with SpyTag-labeled ligands (FIG. 1B).

Example implementations demonstrating the modularity of the disclosed modular functionalized cell membrane-coated nanostructure technology used multiple (e.g., three) different SpyTag-labeled targeting molecules. The example implementations included in vitro and in vivo experimental results showing that each formulation demonstrates affinity towards cells overexpressing the corresponding receptor. For example, using docetaxel as a model drug, it is demonstrated that the enhanced targeting ability significantly increases cytotoxic activity against target cell lines. Also, for example, in a murine tumor model, robust targeting and growth suppression are achieved. The example implementations demonstrates that the disclosed modular design principles can advantageously benefit the CNP platform, which can streamline the introduction of useful functionalities without the need for time-intensive engineering.

Example Embodiments and Implementations of Modular Functionalized CNPs with a SpyTag/SpyCatcher Ligand-Protein Binding System: SpyCatcher-expressing Cells and SpyTag-Labeled Ligands

FIG. 1B shows a diagram illustrating an example method for engineering and characterizing an exemplary modular functionalized CNPs via SpyCatcher-expressing cells and SpyTag-labeled ligands, in accordance with the disclosed technology. The diagram of FIG. 1B shows a process 161 where wild-type cells 160 are engineered to express surface-bound SpyCatcher, which is capable of forming an isopeptide bond with SpyTag. The diagram of FIG. 1B shows a process 163 where the generically-engineered cellular membrane 164 from the SpyCatcher-expressing cells 162 is coated onto nanoparticle cores 166, forming genetically engineered cell membrane-coated nanoparticles 168 (e.g., genetically engineered with SpyCatcher protein in this embodiment, for example). The diagram of FIG. 1B shows a process 165 where the resulting genetically engineered cell membrane-coated nanoparticles can be modularly functionalized with SpyTag-labeled ligands 170 for enhanced functionality, thereby forming modular functionalized, genetically engineered cell membrane-coated nanoparticles 172 (e.g., modular CNPs functionalized with a SpyTag/SpyCatcher ligand-protein binding system (modular SC/ST-functionalized CNPs) in this embodiment, for example). The diagram of FIG. 1B shows a process 167 that employes the exemplary modular SC/ST-functionalized CNPs 172 for cell targeting of multiple target biomarkers in cancer cells 174.

FIGS. 1C-1I show example characterization data from example implementations of the processes 161, 163, 165, and/or 167 and the exemplary modular functionalized CNPs depicted in FIG. 1B. FIG. 1C shows a data plot depicting a flow cytometric analysis of SpyCatcher expression on wild-type HEK293 and SpyCatcher-expressing HEK293 (HEK293-SC) cells. FIG. 1D shows fluorescent images depicting visualization of SpyCatcher expression on wild-type HEK293 and HEK293-SC cells, where Blue: nuclei (DAPI), green: SpyCatcher (sfGFP); and scale bar: 20 μm. FIG. 1E shows western blots probing for SpyTag-labeled ligands in control cell lysate, engineered cell lysate, and purified protein product.

For the example embodiments of modular CNPs functionalized with a SpyTag/SpyCatcher ligand-protein binding system (modular SC/ST-functionalized CNPs), the SpyCatcher003-SpyTag003 binding pair was selected, e.g., due to its ability to rapidly and efficiently form covalent bonds. In the example implementations, the surface of wild-type HEK293 cells was engineered to express SpyCatcher by viral transduction. A transferrin transmembrane domain was used to anchor the protein, which was also fused with super-folder green fluorescent protein (sfGFP) as a reporter. Engineered cells displaying a high density of SpyCatcher were sorted by flow cytometry, followed by monoclonal selection to establish a stable cell line (denoted HEK293-SC). Strong surface expression on HEK293-SC was confirmed by flow cytometry using a fluorescent antibody targeting the SpyCatcher construct (FIG. 1C and FIG. 1F). This was corroborated using fluorescence microscopy to visualize sfGFP signal (FIG. 1D). FIG. 1F shows data plots depicting an example gating strategy used to confirm SpyCatcher surface expression, where cells were gated out from debris, and doublet discrimination was performed using forward scattering (FSC) and side scattering (SSC) plots. Live cells were identified prior to analysis for SpyCatcher expression.

The following soluble ligands were modified with SpyTag: the fluorescent protein mKate2, a designed ankyrin repeat protein (DARPin) targeting mCherry (denoted αmCherry), an affibody targeting epidermal growth factor receptor (EGFR) (denoted αEGFR), and a single-chain variable fragment (scFv) targeting human epidermal growth factor receptor 2 (HER2) (denoted αHER2). Plasmids encoding for each of the constructs were introduced into the appropriate bacterial or mammalian expression system, after which each SpyTag-labeled protein was isolated via an attached histidine tag (His-tag) by Ni-nitriloacetic chromatography. Production and purification of the fusion proteins (denoted ST-mKate2, ST-αmCherry, ST-αEGFR, and ST-αHER2) were optimized using sodium dodecyl sulfate polyacrylamide gel electrophoresis (FIG. 1G) and confirmed using western blotting analysis (FIG. 1E and FIG. 1H). To study the function of αmCherry, an engineered HEK293T cell line expressing mCherry on its surface (denoted HEK293T-mCherry) was developed; SKOV3 ovarian cancer cells (EGFR⁺ and HER2⁺), MDA-MB-453 breast cancer cells (EGFR⁻), and MDA-MB-231 breast cancer cells (HER2⁻) were selected to evaluate the function of αEGFR and αHER2 (FIG. 1I). FIG. 1G shows data plots depicting the purification of SpyTag-labeled ligands, e.g., SDS-PAGE protein bands for ST-mKate2 (a), ST-αmCherry (b), ST-αEGFR (c), and ST-αHER2 (d) in various samples collected during the production process. FIG. 1H shows data plots depicting expression of SpyTag-labeled ligands, e.g., western blots probing for ST-mKate2 (a), ST-αmCherry (b), ST-αEGFR (c), and ST-αHER2 (d) in control cell lysate, engineered cell lysate, and purified protein product. FIG. 1I shows data plots depicting confirmation of biomarker expression on target cell lines. For instance, FIG. 1I shows a gating strategy for confirmation of cell biomarker expression, where cells were gated out from debris, and doublet discrimination was performed using an SSC plot; live cells were identified prior to analysis for mCherry, EGFR, or HER2 expression; and, in panels (b-d), expression of mCherry on wild-type HEK293T and HEK293T-mCherry cells (b), EGFR on MDA-MB-453 and SKOV3 cells (c), and HER2 on MDA-MB-231 and SKOV3 cells (d).

After successful production of the SpyTag-labeled ligands, their ability to conjugate with SpyCatcher and target the appropriate cell surface receptors was evaluated.

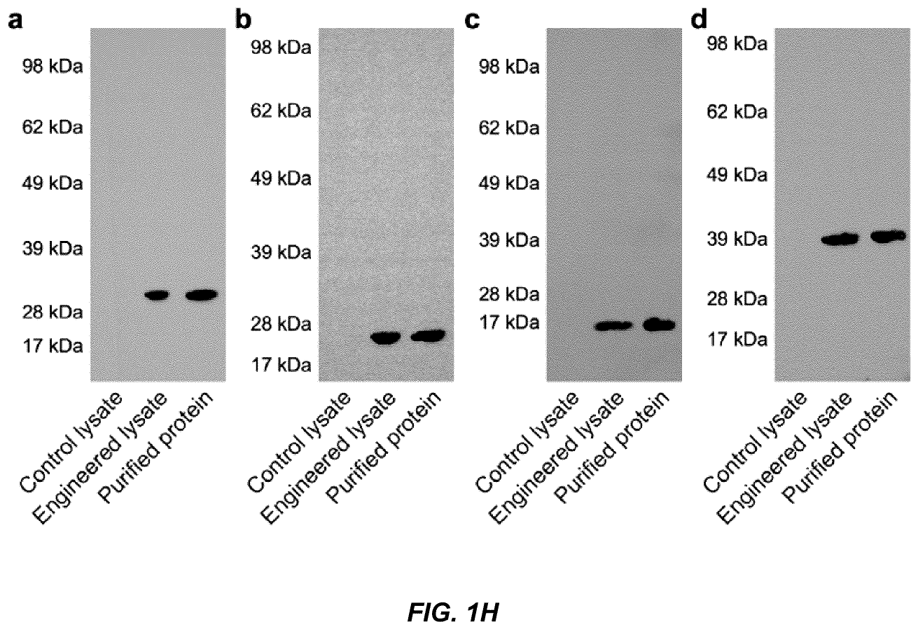

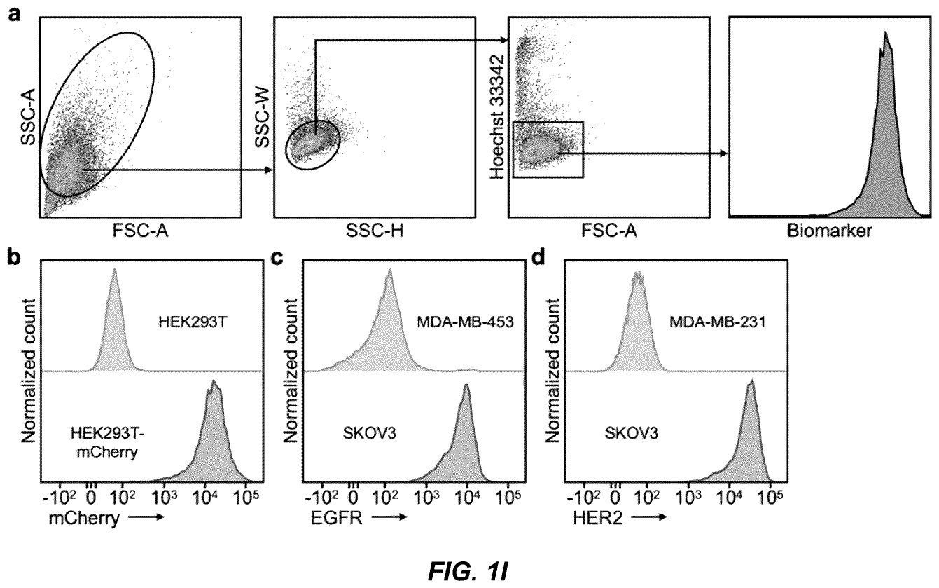

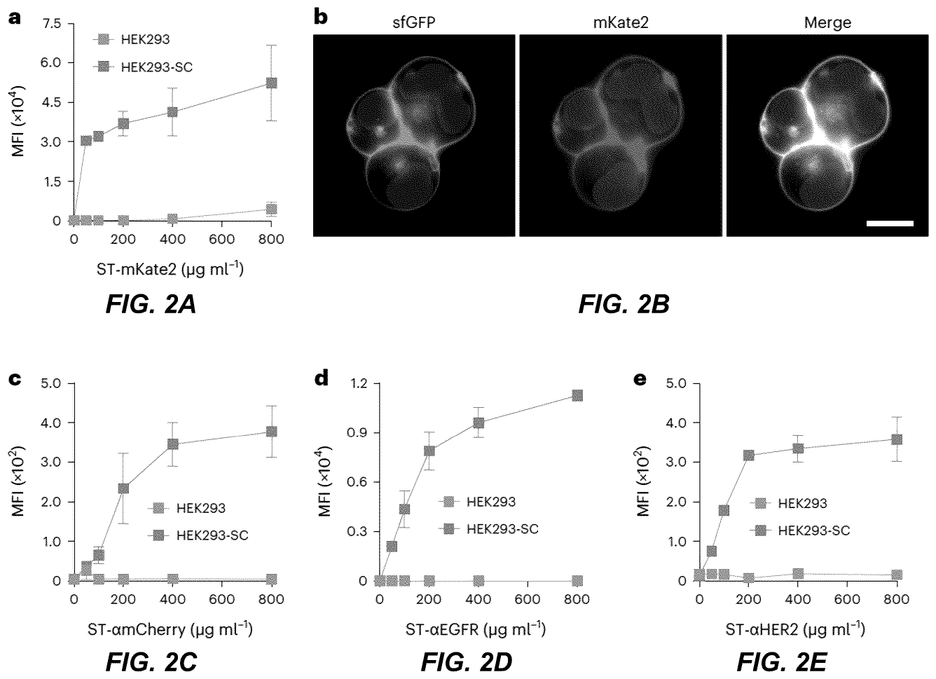

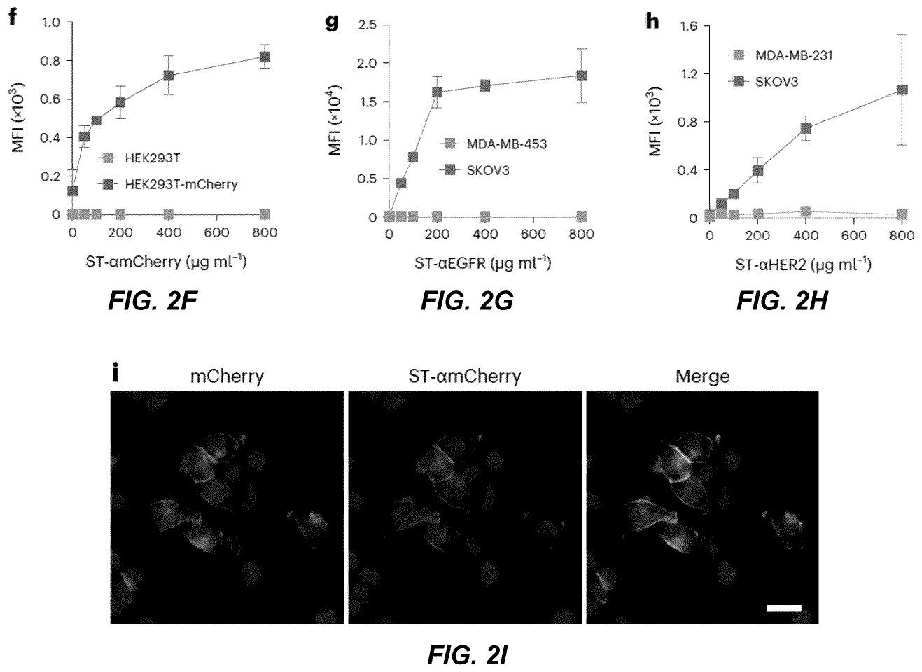

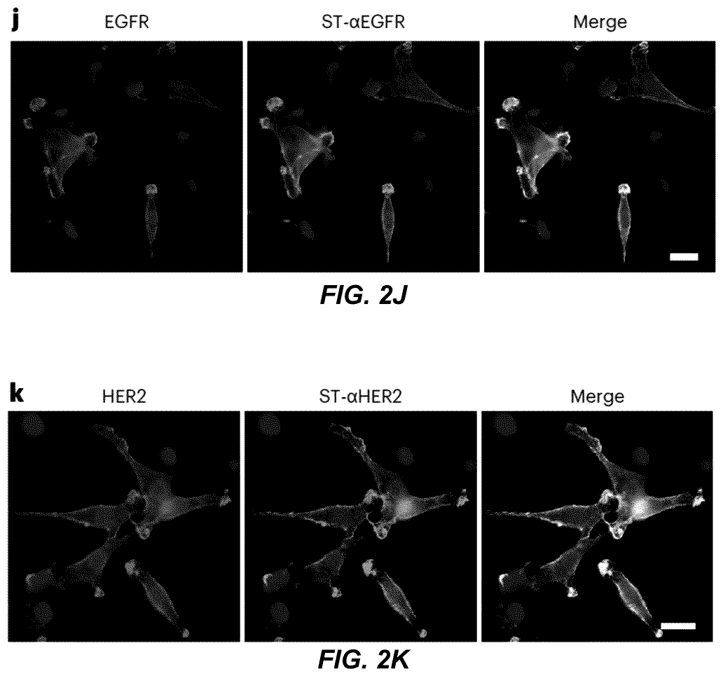

FIGS. 2A-2K show data plots and fluorescent images depicting functional characterization of SpyTag-labeled ligands from example implementations of the disclosed technology. FIG. 2A shows a data plot depicting dose-dependent binding of ST-mKate2 with wild-type HEK293 or HEK293-SC cells (n=3 biological replicates, mean±SD); MFI: mean fluorescence intensity. FIG. 2A shows fluorescent images showing a live cell fluorescent visualization of ST-mKate2 after binding with HEK293-SC cells; blue: nuclei (DAPI); green: SpyCatcher (sfGFP); magenta: mKate2; scale bar: 10 μm. FIGS. 2C-2E show data plots depicting dose-dependent binding of ST-αmCherry (FIG. 2C), ST-αEGFR (FIG. 2D), and ST-αHER2 (FIG. 2E) with wild-type HEK293 or HEK293-SC cells (n=3 biological replicates, mean±SD). FIGS. 2F-2H show data plots depicting dose-dependent binding of ST-αmCherry to wild-type HEK293T or HEK293T-mCherry cells (FIG. 2F), ST-αEGFR to MDA-MB-453 (EGFR−) or SKOV3 (EGFR+) cells (FIG. 2G), and ST-αHER2 to MDA-MB-231 (HER2−) or SKOV3 (HER2+) cells (FIG. 2H) (n=3 biological replicates, mean±SD). FIGS. 21-2J show fluorescent images showing fluorescent visualization of ST-αmCherry (FIG. 2I), ST-αEGFR (FIG. 2J), and ST-αHER2 (FIG. 2K) binding with mixed cultures of HEK293T+HEK293T-mCherry cells (FIG. 2I), MDA-MB-453+SKOV3 cells (FIG. 2J), and MDA-MB-231+SKOV3 cells (FIG. 2K), respectively; blue: nuclei (DAPI); scale bars: 20 μm.

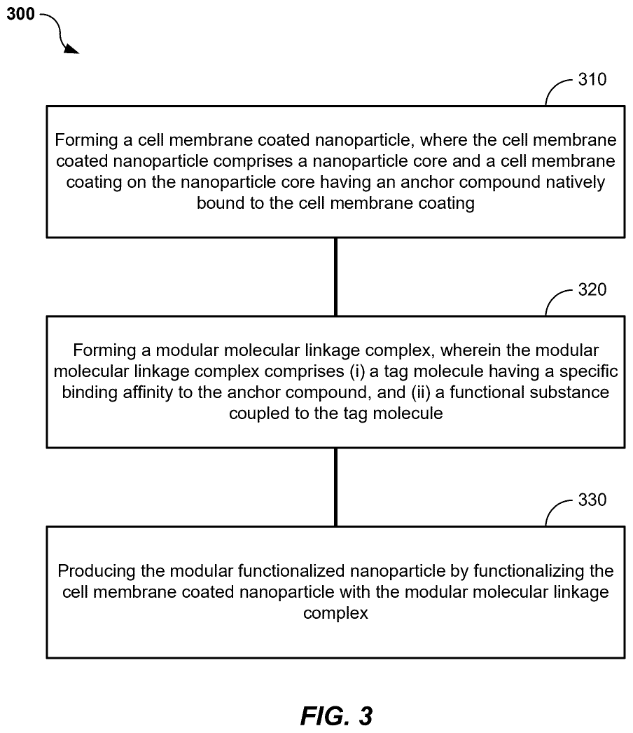

FIG. 3 shows a diagram of an example embodiment of a method 300 for fabricating a modular functionalized nanoparticle, in accordance with the present technology. The method 300 includes a process 310 to form a cell membrane coated nanoparticle, where the cell membrane coated nanoparticle comprises a nanoparticle core and a cell membrane coating on the nanoparticle core having an anchor compound natively bound to the cell membrane coating. The method 300 includes a process 320 to form a modular molecular linkage complex, wherein the modular molecular linkage complex comprises (i) a tag molecule having a specific binding affinity to the anchor compound, and (ii) a functional substance coupled to the tag molecule. The method 300 includes a process 330 to produce the modular functionalized nanoparticle by functionalizing the cell membrane coated nanoparticle with the modular molecular linkage complex. Example embodiments of the method 300 include the method disclosed in connection with FIG. 1B, i.e., example embodiments of a method for engineering and characterizing an exemplary modular functionalized CNPs via SpyCatcher-expressing cells and SpyTag-labeled ligands. Also, example embodiments of the method 300 can include fabrication of modular functionalized nanoparticles including other catcher/tag protein-ligand binding systems, such as SnoopCatcher/SnoopTag and/or DogCatcher/DogTag, or other; and example embodiments of the method 300 can include fabrication of modular functionalized nanoparticles including other coiled-coil peptide binding systems, e.g., including but not limited to the produced modular functionalized nanoparticles comprising anchor compound includes a protein with a leucine zipper motif, and the tag molecule includes a protein or peptide having a corresponding leucine zipper motif.

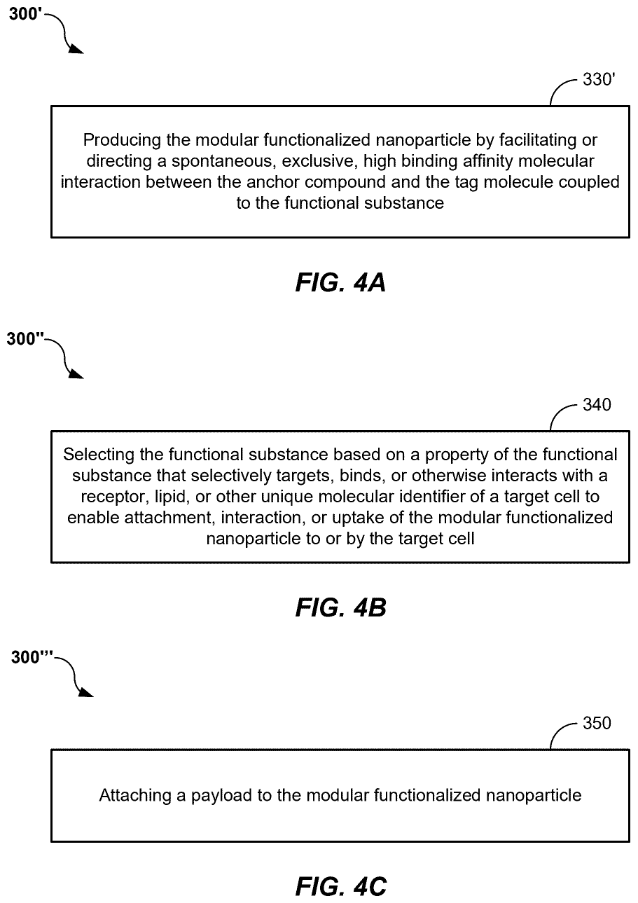

FIGS. 4A-4C show example processes of the example method 300 shown in FIG. 3. FIG. 4A shows a diagram depicting an example embodiment of the process 310 of the method 300, labeled as process 310′ of method 300′, in which the process 310′ includes producing the modular functionalized nanoparticle by facilitating or directing a spontaneous, exclusive, high binding affinity molecular interaction between the anchor compound and the tag molecule coupled to the functional substance. FIG. 4B shows a diagram depicting an example, optional process of the method 300, labeled as process 340 of method 300″, in which the (optional) process 340 includes selecting the functional substance based on a property of the functional substance that selectively targets, binds, or otherwise interacts with a receptor, lipid, or other unique molecular identifier of a target cell to enable attachment, interaction, or uptake of the modular functionalized nanoparticle to or by the target cell. FIG. 4C shows a diagram depicting an example, optional process of the method 300, labeled as process 350 of method 300′″, in which the (optional) process 350 includes attaching a payload to the modular functionalized nanoparticle.

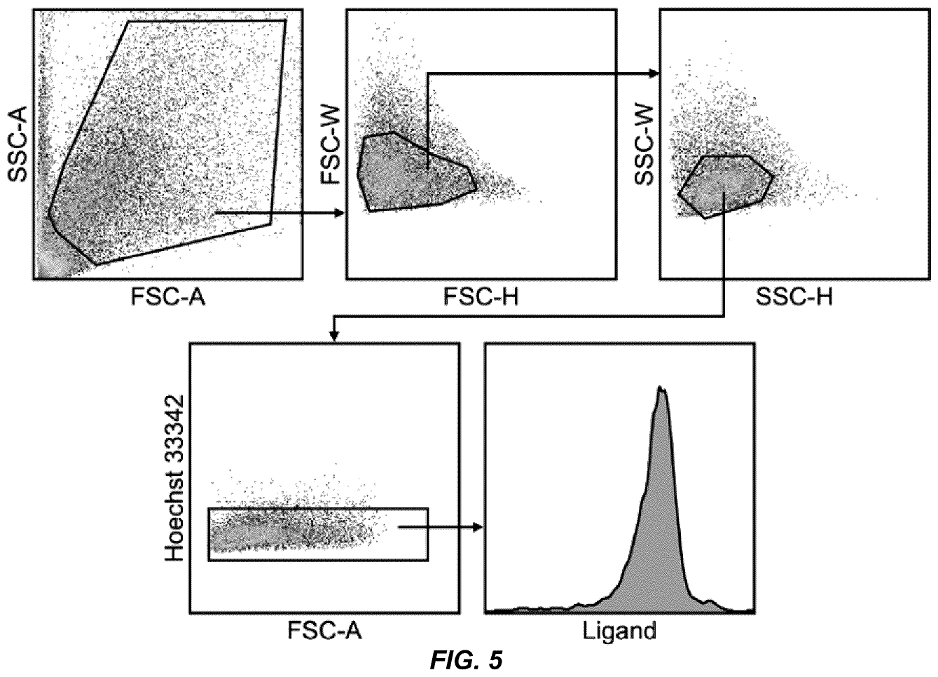

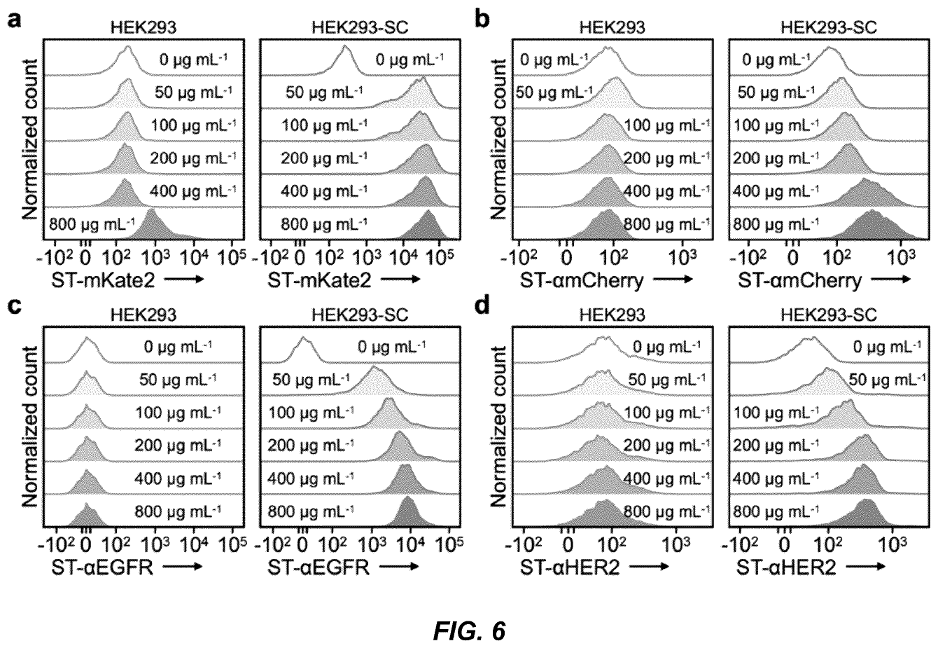

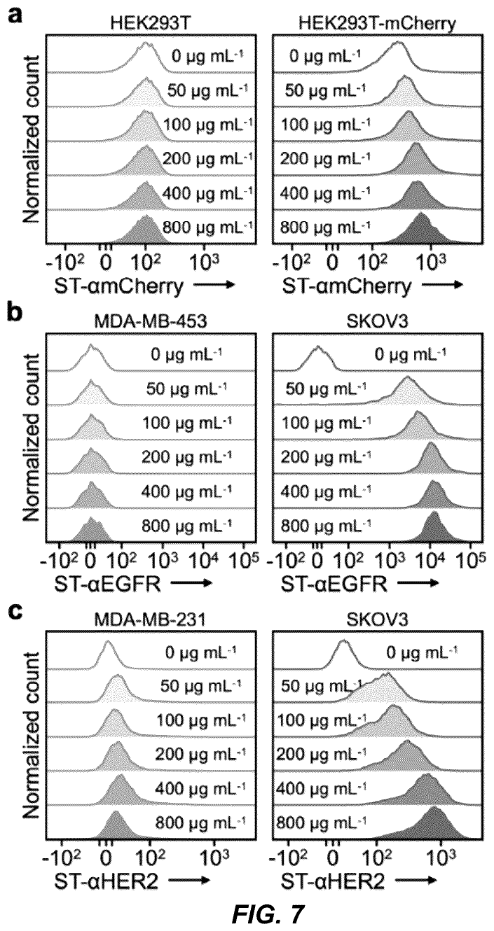

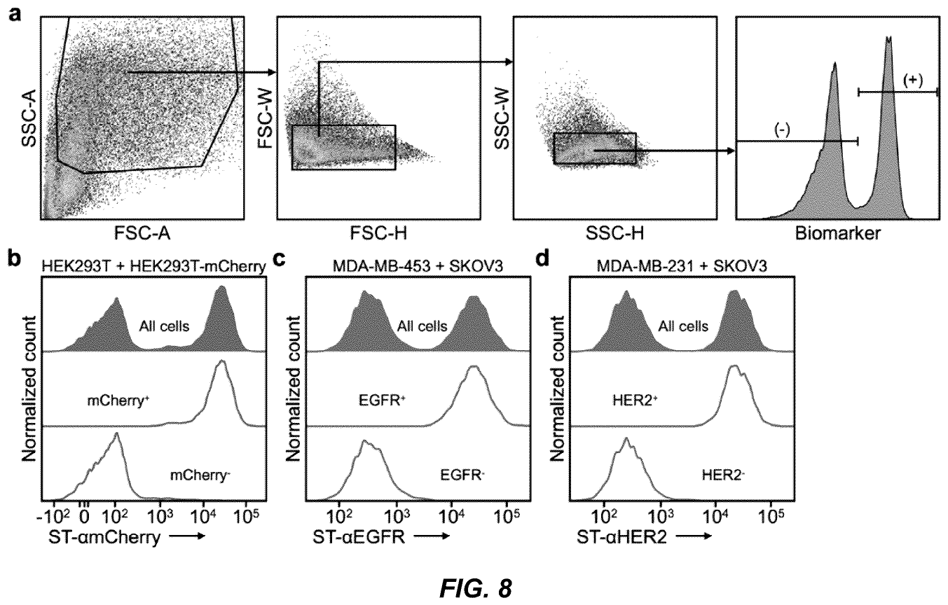

FIGS. 5-8 show data plots and images of example characterization results of SpyTag-labeled ligands and conjugation with SpyCatcher and targeted cell surface receptors. FIG. 5 shows data plots depicting an example gating strategy for functional characterization of SpyTag-labeled ligands, where cells were gated out from debris, and doublet discrimination was performed using FSC and SSC plots, and where live cells were identified prior to analysis for SpyTag-labeled ligand binding or targeting. FIG. 6 shows data plots depicting in vitro binding of SpyTag-labeled ligands with SpyCatcher-expressing cells, i.e., representative flow histograms of dose-dependent binding of ST-mKate2 (a), ST-αmCherry (b), ST-αEGFR (c), and ST-αHER2 (d) with wild-type HEK293 or HEK293-SC cells. FIG. 7 shows data plots depicting in vitro binding in vitro binding of SpyTag-labeled ligands with target cells, i.e., representative flow histograms of dose-dependent targeting of ST-αmCherry to wild-type HEK293T or HEK293T-mCherry cells (a), ST-αEGFR to MDA-MB-453 (EGFR−) or SKOV3 (EGFR+) cells (b), and ST-αHER2 to MDA-MB-231 (HER2−) or SKOV3 (HER2+) cells (c). FIG. 8 shows data plots depicting in vitro binding of SpyTag-labeled ligands in cell co-cultures. FIG. 8 (panel a) shows an example gating strategy for distinguishing positive biomarker expression in co-cultures, which cells were first gated out from debris, and then doublet discrimination was performed based on FSC and SSC plots. The resulting cell population was divided based on mCherry, EGFR, or HER2 expression prior to assessing the targeting of ST-αmCherry, ST-αEGFR, or ST-αHER2. FIG. 8 (panels b-d) show representative flow histograms for ST-αmCherry binding to various cell populations in an HEK293T and HEK293T-mCherry co-culture (b), ST-αEGFR binding to various cell populations in an MDA-MB-453 (EGFR−) and SKOV3 (EGFR+) co-culture (c), and ST-αHER2 binding to various cell populations in an MDA-MB-231 (HER2−) and SKOV3 (HER2+) co-culture (d).

To confirm the function of SpyTag, ST-mKate2 was incubated at increasing concentrations with HEK293-SC (FIG. 2A and FIG. 5 and FIG. 6A). After washing away unbound ligands, dose-dependent binding was observed when analyzing the cells by flow cytometry. Strong mKate2 fluorescence was observed after incubation with HEK293-SC (FIG. 2B). Most of the signal was visualized on the periphery of the cells and colocalized with the sfGFP reporter, supporting that the SpyTag-labeled ligands were being complexed with surface-bound SpyCatcher. Similar dose-dependent binding was observed for the other fusion proteins (FIG. 2C-2E and FIGS. 6B-6D). To evaluate the functionality of the targeting constructs, each SpyTag-labeled ligand was incubated with cells expressing its cognate receptor (FIG. 2F-2H and FIG. 7). ST-αmCherry efficiently bound to HEK293T-mCherry cells but not to wild-type HEK293T cells. ST-αEGFR and ST-αHER2 both bound to SKOV3 cells in a dose-dependent manner, but not to MDA-MB-453 and MDA-MB-231 cells, respectively. To further validate targeting specificity, each SpyTag-labeled ligand was incubated with a heterogeneous population of cells both positive and negative for the cognate receptor (FIG. 2I-2K and FIG. 8). In each case, the ligand bound near exclusively to the appropriate target cell.

### Example Synthesis and Characterization of Modular CNPs

Having successfully confirmed the functionality of all engineered ligands, example implementations were conducted that demonstrate the modular modification of scalable, functionalized CNPs.

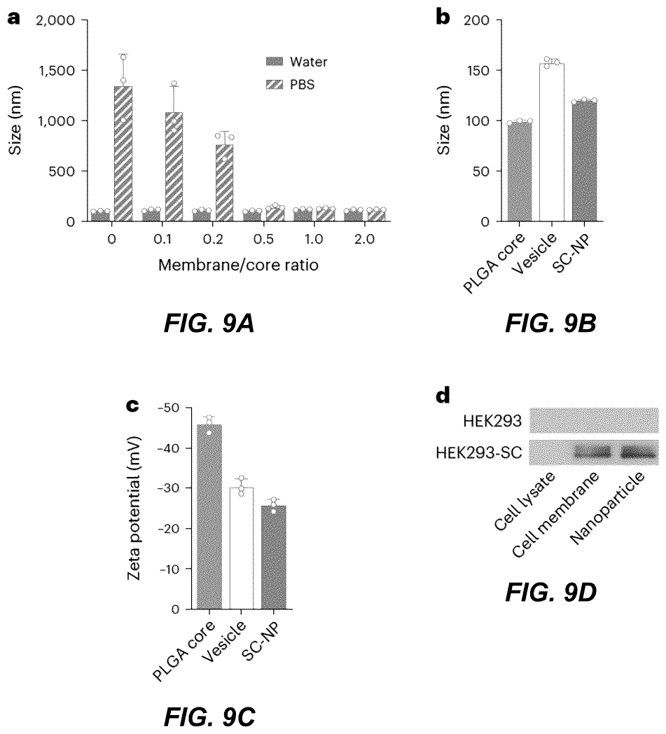

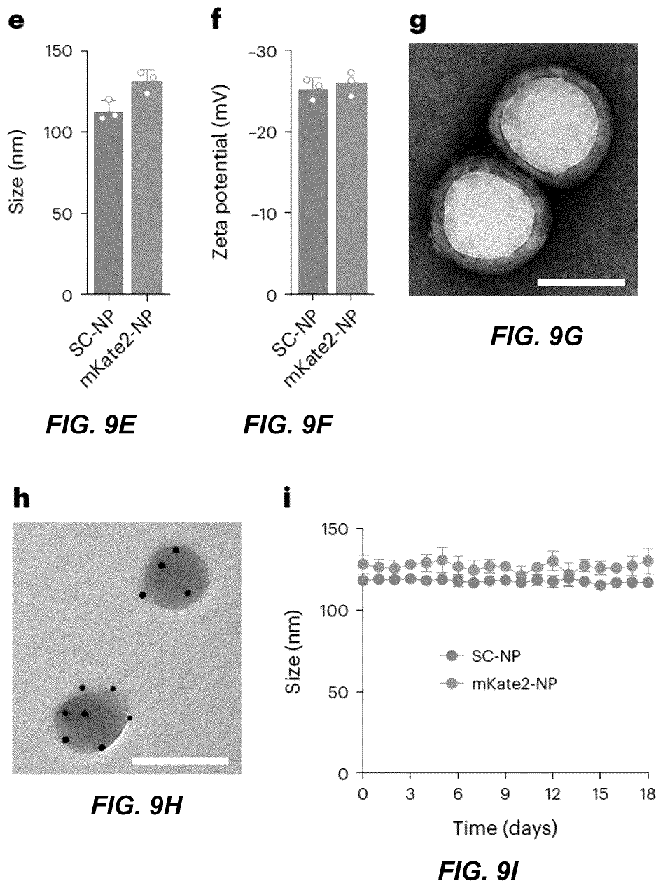

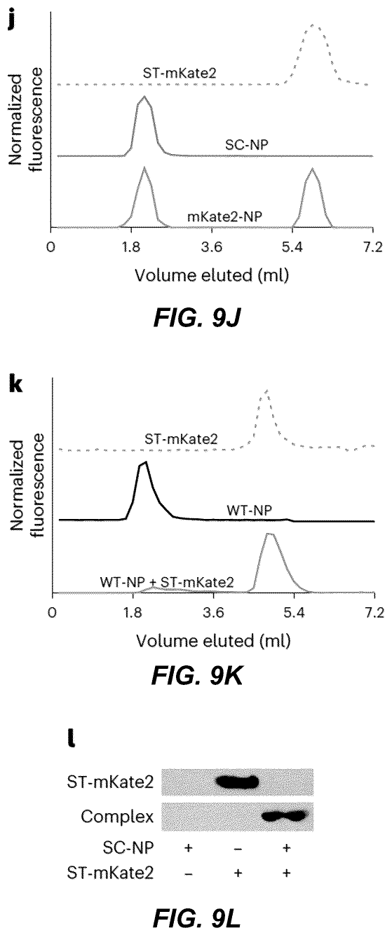

FIG. 9A-9L show data plots and images from example implementations of nanoparticle synthesis and functionalization techniques in accordance with the present technology. FIG. 9A shows a data plot depicting size data of example SC-NPs fabricated at varying membrane protein to PLGA core weight ratios in water or PBS (n=3 technical replicates, mean+SD). FIGS. 9B and 9C show data plots depicting the hydrodynamic diameter (FIG. 9B) and surface zeta potential (FIG. 9C) of example bare PLGA cores, HEK293-SC membrane vesicles, and SC-NPs (n=3 technical replicates, mean+SD). FIG. 9D shows an image of western blots probing for SpyCatcher on cell lysate, cell membrane, and membrane-coated nanoparticles derived from wild-type HEK293 or HEK293-SC cells. FIGS. 9E and 9F show data plots depict the hydrodynamic diameter (FIG. 9E) and surface zeta potential (FIG. 9F) of SC-NPs and mKate2-NPs (n=3 technical replicates, mean+SD). FIG. 9G shows a transmission electron micrograph (TEM) image of mKate2-NPs negatively stained with uranyl acetate; scale bar: 100 nm. FIG. 9H shows an immunogold TEM image of mKate2-NPs probing for ST-mKate2; scale bar: 100 nm. FIG. 9I shows a data plot depicting the size of example SC-NPs and mKate2-NPs in an isotonic sucrose solution over 18 days (n=3 technical replicates, mean+SD). FIG. 9J shows a data plot of fluorescence intensity data of fractions collected from the size exclusion chromatography of ST-mKate2 (mKate2), SC-NPs (DiR), and mKate2-NPs (mKate2) prior to purification. FIG. 9K shows a data plot of fluorescence intensity data of fractions collected from the size exclusion chromatography of ST-mKate2 (mKate2), WT-NPs (DiR), and WT-NPs+ST-mKate2 (mKate2). FIG. 9L shows an image of western blots probing for ST-mKate2 or SpyCatcher-ST-mKate2 complex in samples containing various combinations of SC-NPs and ST-mKate2.

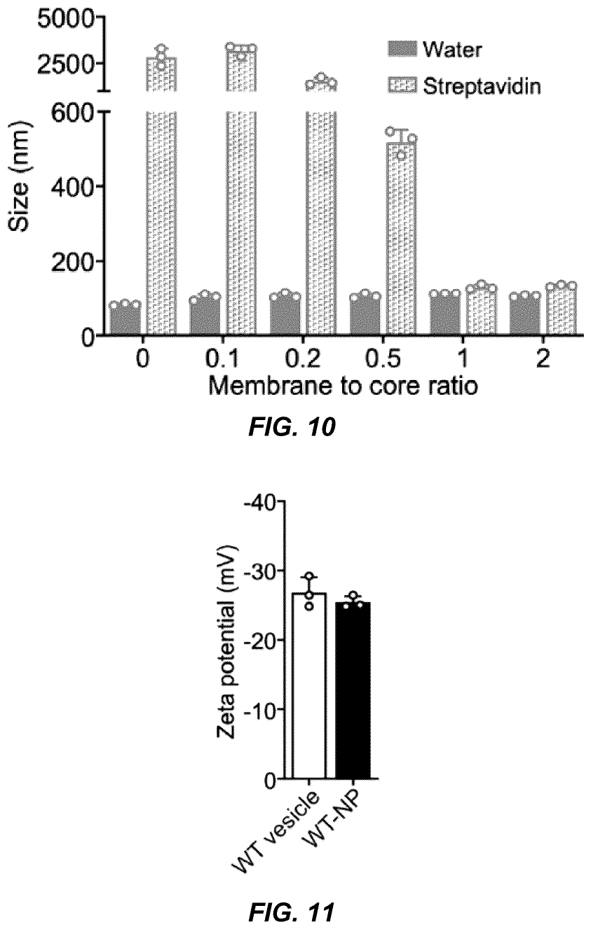

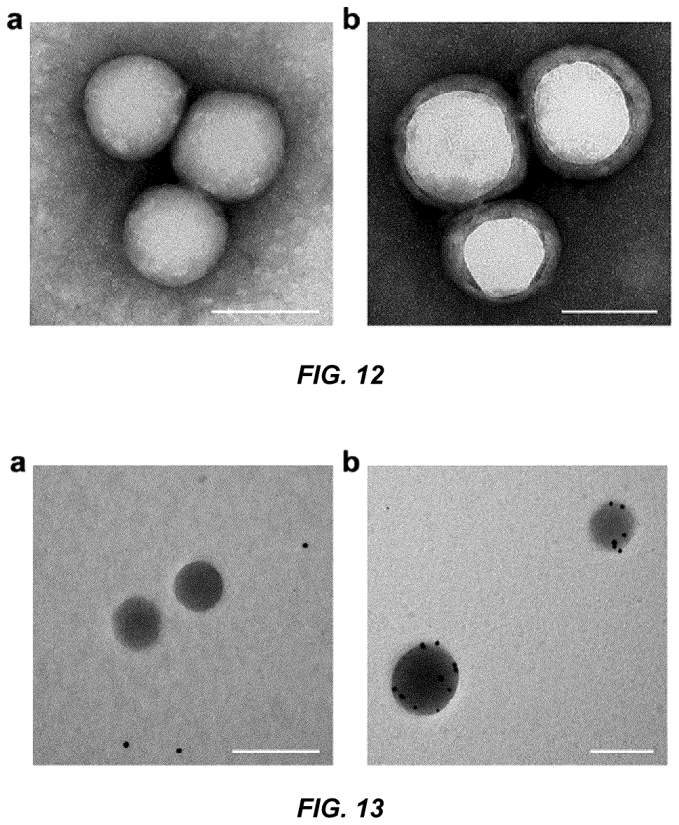

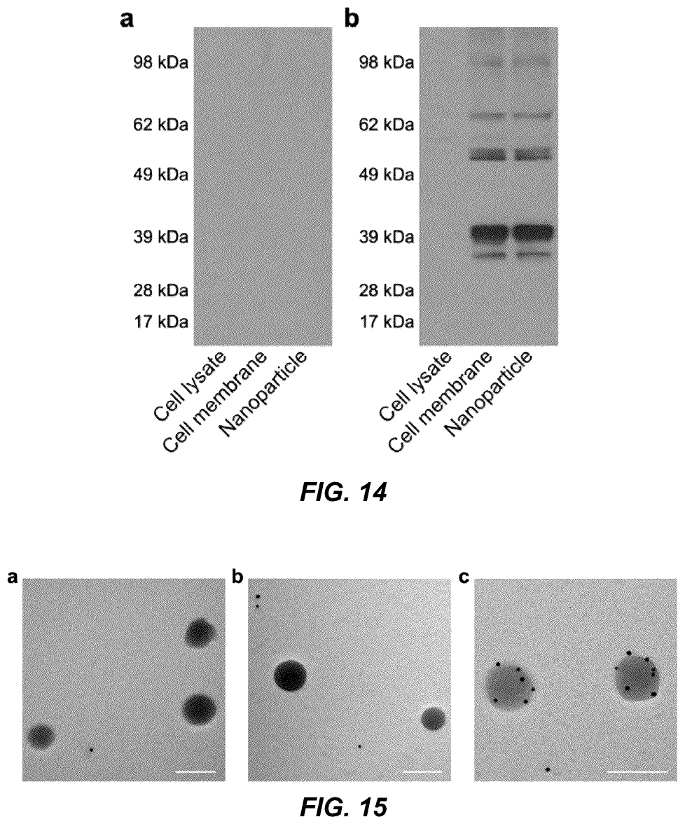

FIGS. 10-15 show data plots and images of example characterization results of fabrication of modular, scalable CNPs. FIG. 10 shows a data plot of the confirmation of membrane coverage on CNPs, i.e., the size of biotinylated PLGA cores when coated with HEK293-SC membrane at varying protein to PLGA core weight ratios before and after incubation with streptavidin (n=3, mean+SD). FIG. 11 shows a data plot of the zeta potential of WT-NPs. Surface zeta potential of wild-type HEK293 membrane vesicles (WT vesicles) and WT-NPs (n=3, mean+SD). FIG. 12 shows TEM images of bare PLGA cores (a) and SC-NP morphology (b), i.e., negatively stained with uranyl acetate; scale bars: 100 nm. FIG. 13 shows images showing confirmation of right-side-out membrane coating, where immunogold TEM images depict bare PLGA cores (a) and SC-NPs (b) probing for an extracellular region of Na+/K+−ATPase; scale bars: 100 nm. FIG. 14 shows data confirming SpyCatcher retention, i.e., western blots probing for SpyCatcher in cell lysate, cell membrane, and membrane-coated nanoparticles derived from wild-type HEK293 (a) or HEK293-SC (b) cells. FIG. 15 shows data confirming SpyCatcher presentation, i.e., immunogold TEM images of bare PLGA cores (a), WT-NPs (b), and SC-NPs (c) probing for the SpyCatcher construct; scale bars: 100 nm.

In the example implementations, the plasma membrane from HEK293-SC cells was isolated and then coated onto the surface of poly(lactic-co-glycolic acid) (PLGA) nanoparticle cores by sonication, thus yielding the base SpyCatcher-functionalized nanoparticles (denoted SC-NPs). SC-NP synthesis was optimized by varying the amount of membrane to PLGA cores; beginning at a 1:2 protein to polymer weight ratio, the membrane coverage was sufficient to prevent nanoparticle aggregation in phosphate buffered saline (PBS) due to charge screening (FIG. 9A). This coating ratio was used to fabricate SC-NPs in the subsequent studies. In some embodiments, for example, the protein to polymer weight ratio can be in a range of about 1:8, or about 1:7, or about 1:6, or about 1:5, or about 1:4, or about 1:3, or about 1:2, or about 1:1. Whereas, in some embodiments, for example, the protein to polymer weight ratio can be in a range of about 8:1, or about 7:1, or about 6:1, or about 5:1, or about 4:1, or about 3:1, or about 2:1, or about 1:1. The completeness of the coating was confirmed using biotinylated PLGA cores, which can be crosslinked in the presence of streptavidin (FIG. 10). Using dynamic light scattering, SC-NPs were measured to be approximately 120 nm, a slight increase from the PLGA cores (FIG. 9B). The surface zeta potential of SC-NPs was similar to that of HEK293-SC membrane vesicles, further suggesting successful membrane coating (FIG. 9C). The zeta potential of SC-NPs was also similar to that of nanoparticles coated with wild-type HEK293 membrane (denoted WT-NPs), indicating that the introduction of SpyCatcher did not have an impact on surface charge (FIG. 11). Due to the complex nature of cell membrane, its net negative charge does not preclude successful coating around a negatively charged core. For visualization, SC-NPs were subjected to transmission electron microscopy (TEM), which revealed a core-shell structure that is characteristic of membrane-coated nanoparticles and not present on bare PLGA cores (FIG. 12). Immunogold TEM probing for the extracellular region of Na⁺/K⁺-ATPase, a plasma membrane marker, demonstrated that the membrane was coated in a right-side-out orientation (FIG. 13). Western blotting was used to confirm the presence of SpyCatcher on the final SC-NP formulation (FIG. 9D and FIG. 14). The proper presentation of SpyCatcher on SC-NPs was further corroborated by immunogold TEM (FIG. 15).

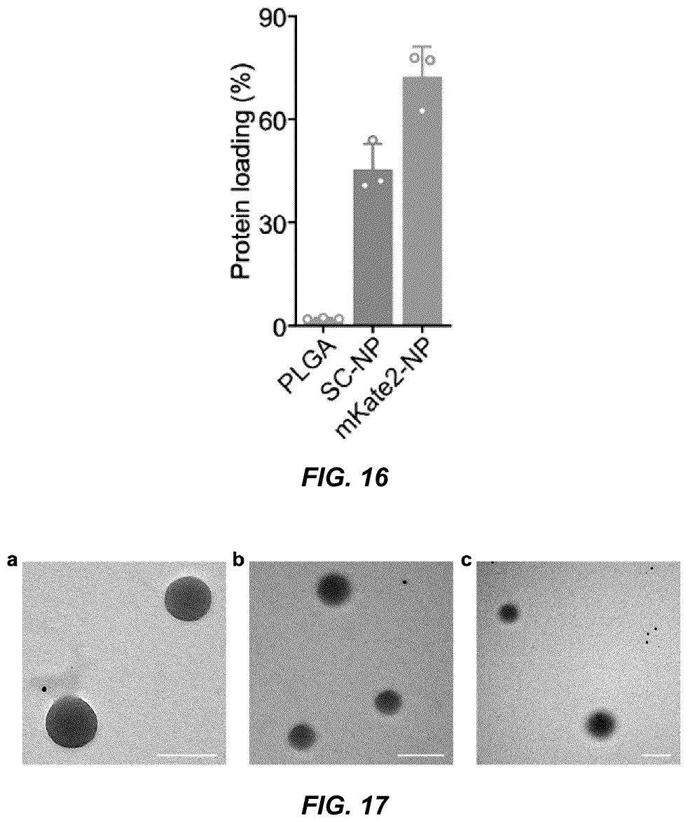

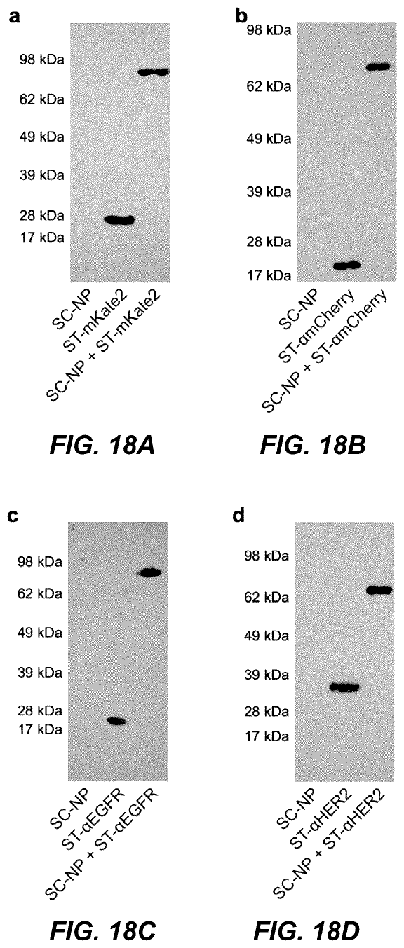

FIGS. 16-18D show data plots and images of example characterization results of cell membrane functionalization on modular, scalable CNPs. FIG. 16 shows example data of a quantification of membrane functionalization onto nanoparticles, depicting the quantification of protein on bare PLGA cores, SC-NPs, and mKate2-NPs relative to PLGA weight (n=3, mean+SD). FIG. 17 shows images demonstrating controls for the confirmation of nanoparticle functionalization with SpyTag-labeled ligands, i.e., immunogold TEM images of bare PLGA cores (a), WT-NPs (b), and SC-NPs (c) probing for ST-mKate2; scale bars: 100 nm. FIGS. 18A-18D show images of example results for covalent linking of SpyTag-labeled ligands to SC-NPs, including western blots probing for the complexation of ST-mKate2 (a), ST-αmCherry (b), ST-αEGFR (c), and ST-αHER2 (d) with SC-NPs.

ST-mKate2 was selected to preliminarily demonstrate the modular functionalization of SC-NPs. To synthesize mKate2-conjugated nanoparticles (denoted mKate2-NPs), SC-NPs were incubated with the soluble protein, followed by purification using size exclusion chromatography (SEC). After functionalization, nanoparticle size increased slightly, while the zeta potential remained similar (FIGS. 9E and 9F). The protein loading also increased on mKate2-NPs versus SC-NPs (FIG. 16). TEM imaging revealed no major changes in morphology (FIG. 9G), and immunogold staining was used to confirm the proper presentation of ST-mKate2 on the nanoparticle surface (FIG. 9H and FIG. 17). Both SC-NPs and mKate2-NPs were stable in isotonic sucrose over 18 days (FIG. 91). To confirm the binding between ST-mKate2 with SC-NPs, a mixture of the two components following co-incubation was subjected to SEC. When measuring mKate2 fluorescence, two distinct peaks were observed. The first peak eluted at the same time as SC-NPs, which were labeled with a far-red fluorescent dye, providing evidence for the successful formation of mKate2-NPs; the second peak corresponded with excess ST-mKate2 (FIG. 9J). In contrast, when ST-mKate2 was incubated with WT-NPs, there was only one distinct peak when measuring mKate2 fluorescence, corresponding to free ST-mKate2 (FIG. 9K). Irreversible binding of the ligand to SC-NPs was assessed by western blotting under denaturing conditions (FIG. 9L and FIG. 18A). When probing for the His-tag on the ST-mKate2 construct, only a lower molecular weight band was observed when no SC-NPs were present; however, when ST-mKate2 was incubated with SC-NPs, a higher molecular weight band appeared with a concomitant decrease in the intensity of the lower band, indicating that ST-mKate2 had been covalently conjugated to SpyCatcher. Similar results were observed for each of the other SpyTag-labeled ligands (FIGS. 18B-18D).

### In Vitro Cancer Cell Targeting

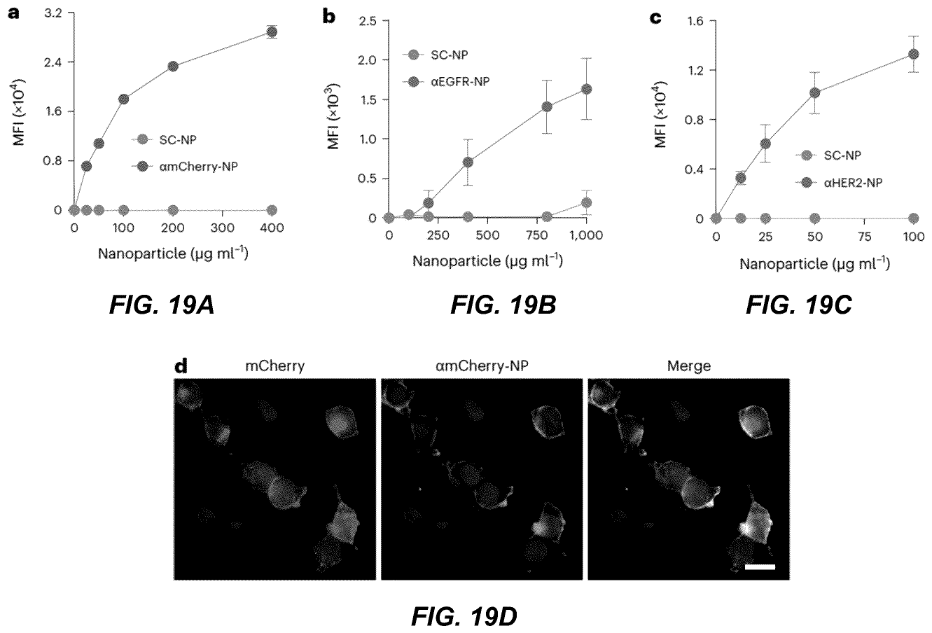

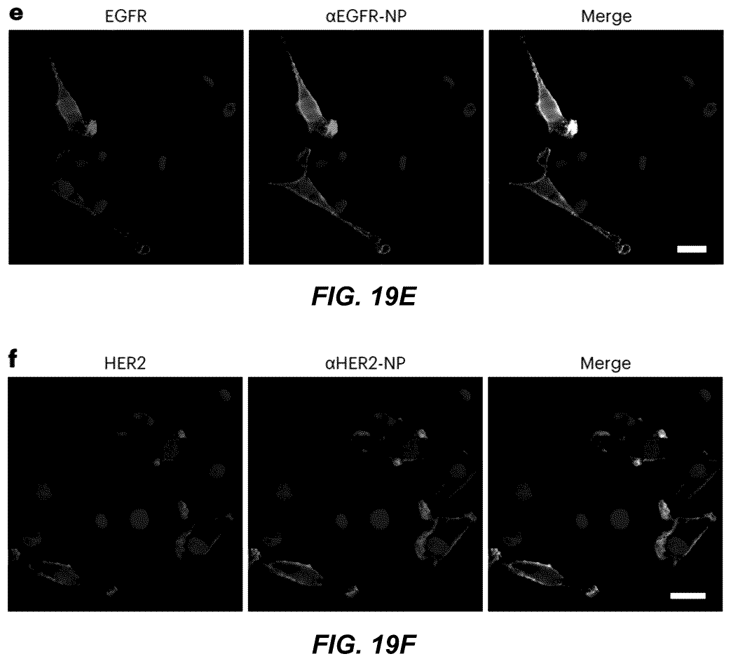

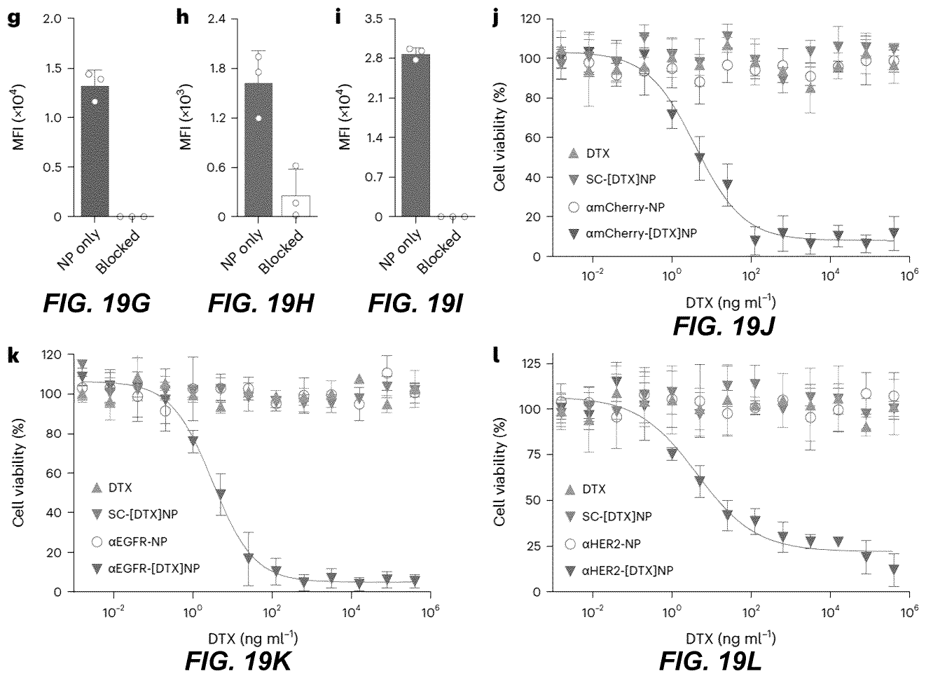

FIG. 19A-19L show data plots and images from example implementations demonstrating functional characterization of modularly functionalized nanoparticles, in accordance with the present technology. FIGS. 19A-19C show data plots depicting dose-dependent binding of αmCherry-NPs to HEK293T-mCherry cells (a), αEGFR-NPs to SKOV3 cells (b), and αHER2-NPs to SKOV3 cells (c) (n=3 biological replicates, mean±SD); non-targeted SC-NPs were used as controls. MFI: mean fluorescence intensity. FIGS. 19D-19F show fluorescent images depicting fluorescent visualization of αmCherry-NP (FIG. 19D), αEGFR-NP (FIG. 19E), and αHER2-NP (FIG. 19F) binding with mixed cultures of HEK293T+HEK293T-mCherry cells (FIG. 19D), MDA-MB-453+SKOV3 cells (FIG. 19E), and MDA-MB-231+SKOV3 cells (FIG. 19F), respectively; where blue: nuclei (DAPI); and scale bars: 20 μm. FIGS. 19G-19I show data plots depicting the binding of αmCherry-NPs to HEK293T-mCherry cells (FIG. 19G), αEGFR-NPs to SKOV3 cells (FIG. 19H), and αHER2-NPs to SKOV3 cells (FIG. 191) in the presence of the corresponding free ligand as a blocking agent (n=3 biological replicates, mean+SD). FIGS. 19J-19L show data plots depicting dose-dependent cytotoxicity of αmCherry-[DTX]NPs against HEK293T-mCherry cells (FIG. 19J), αEGFR-[DTX]NPs against SKOV3 cells (FIG. 19K), and αHER2-[DTX]NPs against SKOV3 cells (FIG. 19L) measured at 72 h after 15 min of co-incubation (n=3 biological replicates, mean±SD); free DTX, non-targeted SC-[DTX]NPs, and the respective unloaded nanoparticles (αmCherry-NPs, αEGFR-NPs, and αHER2-NPs) were used as controls.

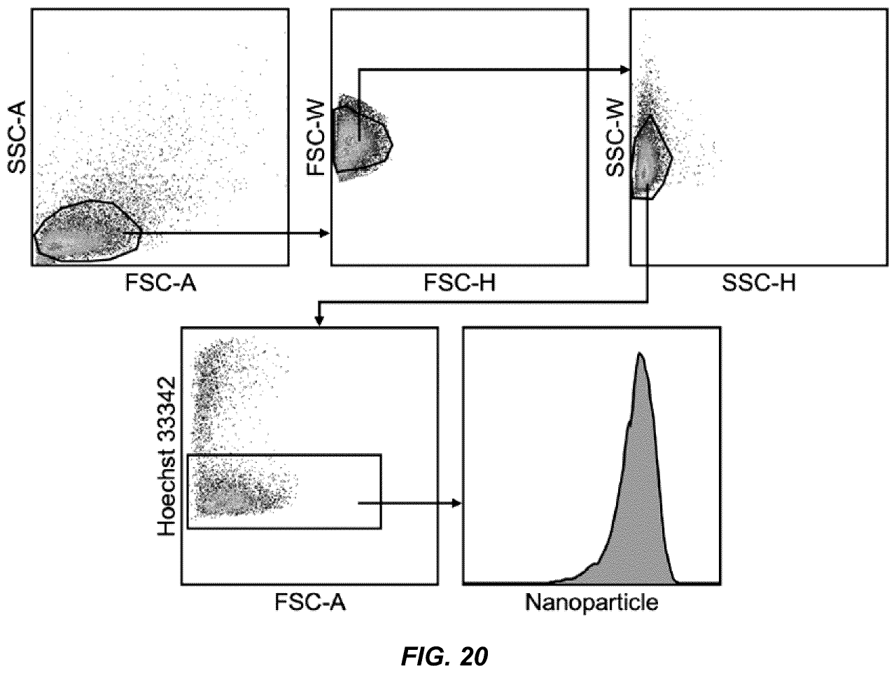

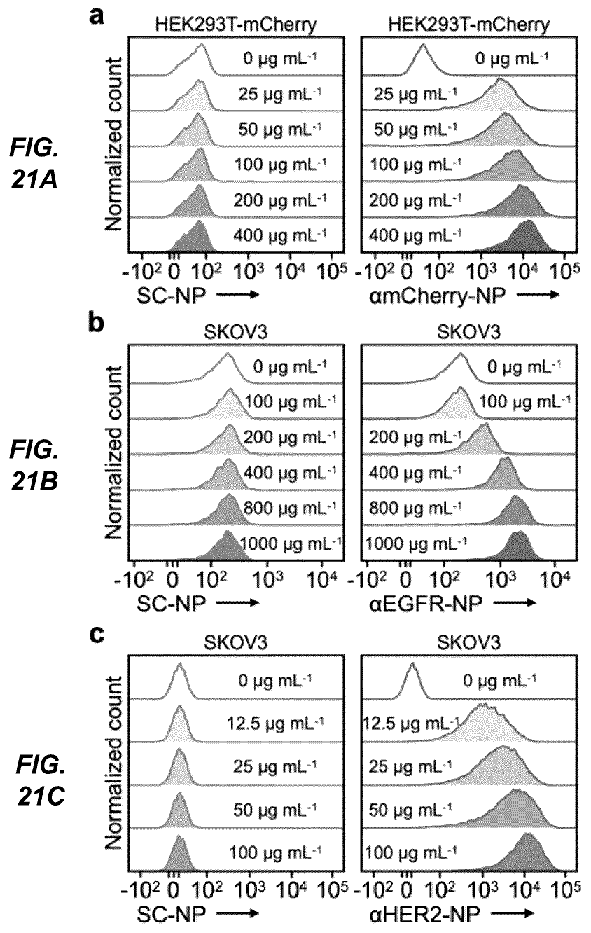

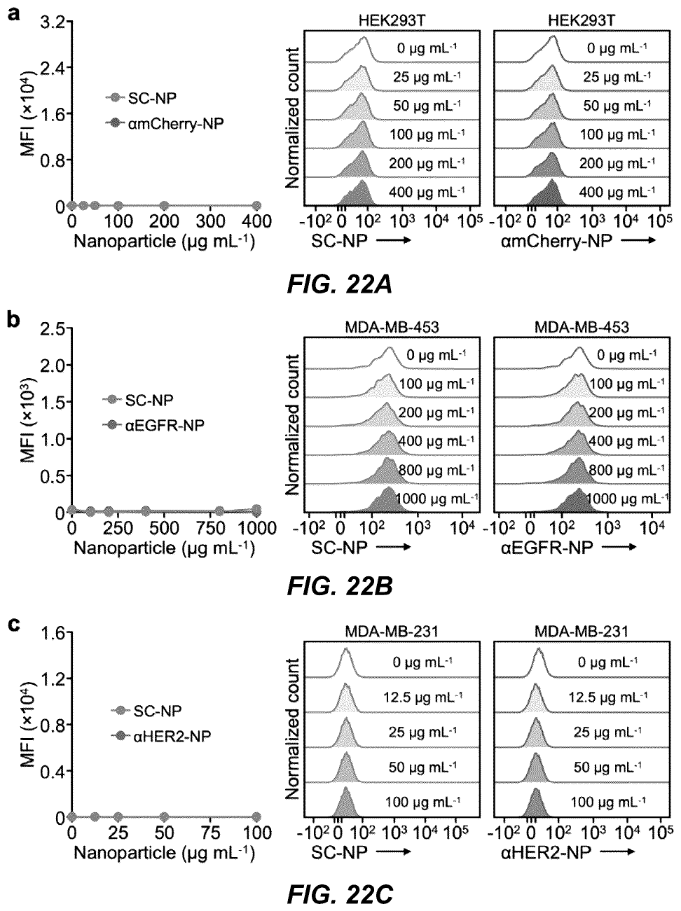

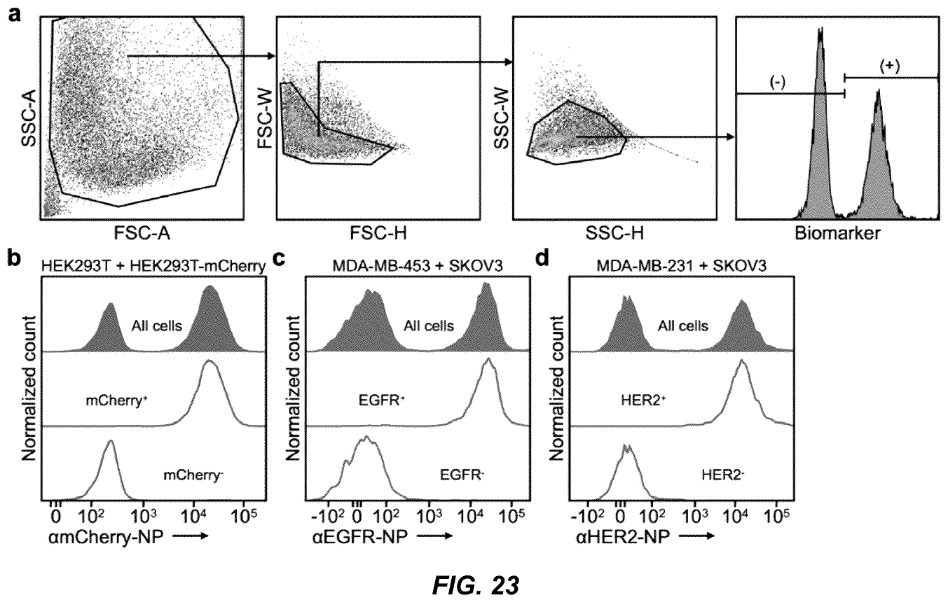

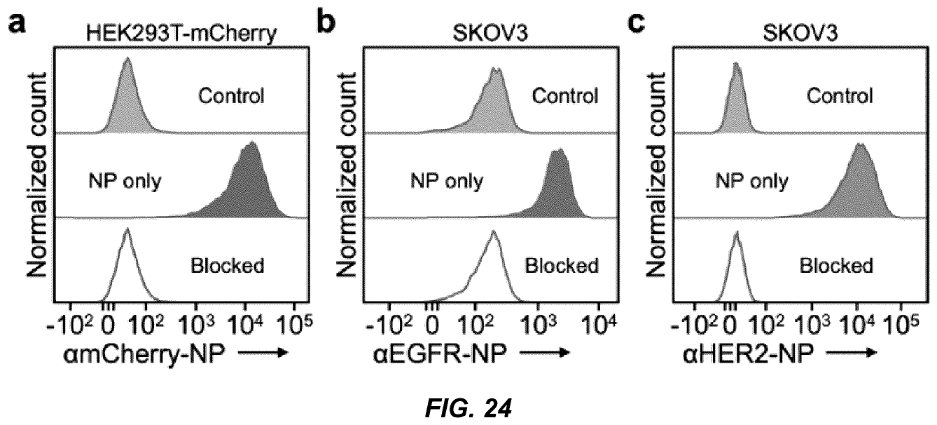

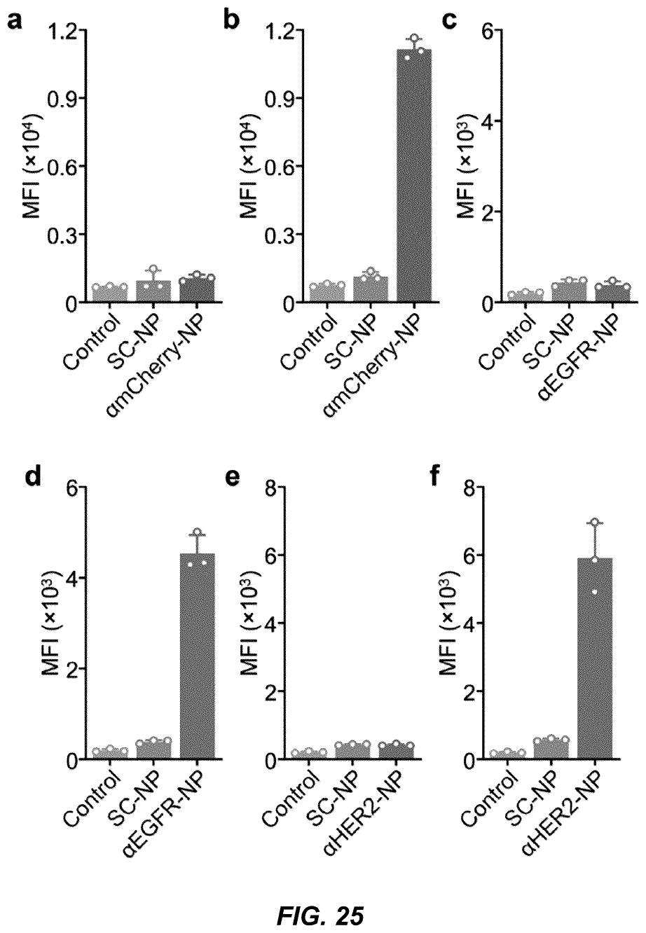

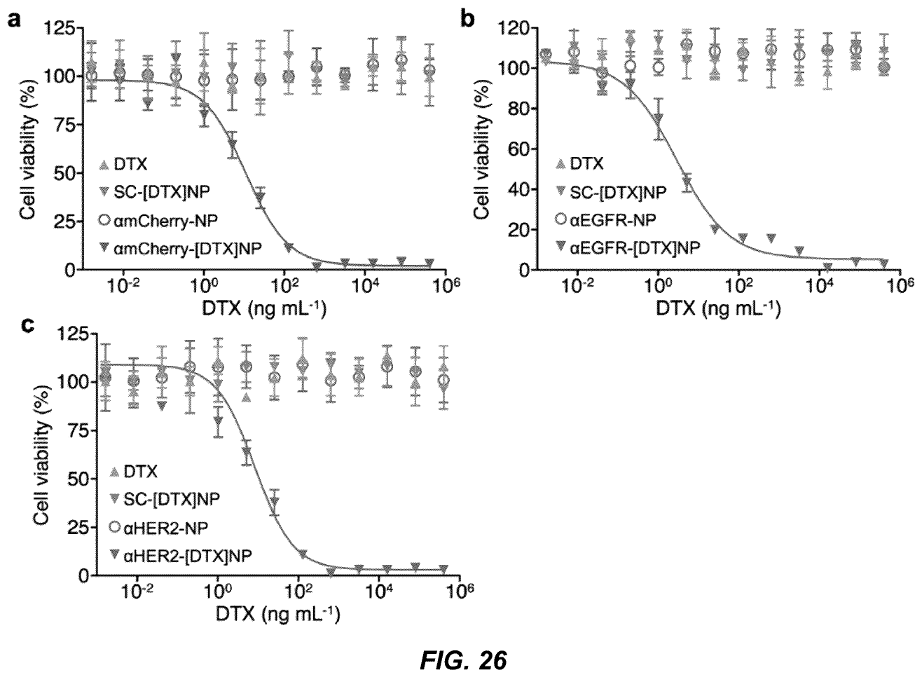

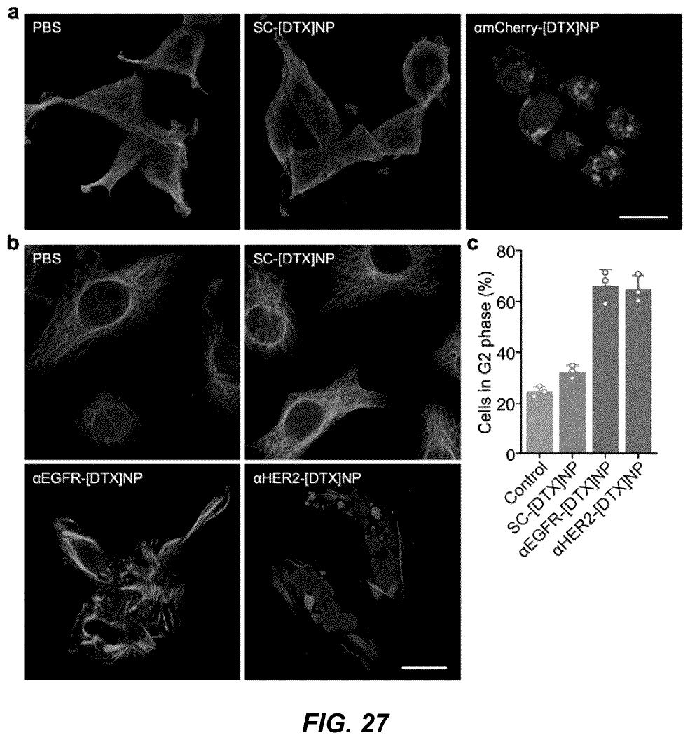

FIGS. 20-27 show data plots and images of example in vitro characterization results of modular functionalization on cell membrane-coated CNPs and target cell binding, in accordance with the present technology. FIG. 20 shows data plots depicting a gating strategy for detection of nanoparticle targeting, where cells were gated out from debris, and doublet discrimination was performed using FSC and SSC plots; and where live cells were identified prior to analysis for nanoparticle binding. FIGS. 21A-21C show data plots depicting the in vitro binding of modularly functionalized nanoparticles with target cells, i.e., representative flow histograms of dose-dependent targeting of αmCherry-NPs to HEK293T-mCherry cells (FIG. 21A), αEGFR-NPs to SKOV3 cells (FIG. 21B), and αHER2-NPs to SKOV3 cells (FIG. 21CA), where non-targeted SC-NPs were used as controls. FIGS. 22A-22C show data plots depicting the in vitro binding of modularly functionalized nanoparticles with control cells, i.e., dose-dependent binding of αmCherry-NPs to wild-type HEK293T cells (FIG. 22A), αEGFR-NPs to MDA-MB-453 cells (FIG. 22B), and αHER2-NPs to MDA-MB-231 cells (FIG. 22C) (n=3, mean±SD), where non-targeted SC-NPs were used as controls, and where MFI: mean fluorescence intensity. FIG. 23 shows data plots depicting the in vitro binding of modularly functionalized nanoparticles in cell co-cultures. Panel (a) of FIG. 23 shows a gating strategy for distinguishing positive biomarker expression in co-cultures, where cells were first gated out from debris, and then doublet discrimination was performed based on FSC and SSC plots, and where the resulting cell population was divided based on mCherry, EGFR, or HER2 expression prior to assessing the targeting of αmCherry-NPs, αEGFR-NPs, or αHER2-NPs. Panels (b)-(d) of FIG. 23 shows representative flow histograms for αmCherry-NP binding to various cell populations in an HEK293T and HEK293T-mCherry co-culture (b), αEGFR-NP binding to various cell populations in an MDA-MB-453 (EGFR−) and SKOV3 (EGFR+) co-culture (c), and αHER2-NP binding to various cell populations in an MDA-MB-231 (HER2-) and SKOV3 (HER2+) co-culture (d). FIG. 24 shows data plots depicting the targeting effect of modularly functionalized nanoparticles in the presence of blocking, i.e., binding of αmCherry-NPs to HEK293T-mCherry cells (a), αEGFR-NPs to SKOV3 cells (b), and αHER2-NPs to SKOV3 cells (c) in the presence of the corresponding free ligand as a blocking agent. FIG. 25 shows data plots depicting internalization of modularly functionalized nanoparticles by target cells, i.e., internalization of αmCherry-NPs by wild-type HEK293T (a) or HEK293T-mCherry (b) cells, αEGFR-NPs by MDA-MB-453 (EGFR-) (c) or SKOV3 (EGFR+) (d) cells, and αHER2-NPs by MDA-MB-231 (HER2-) (e) or SKOV3 (HER2+) (f) cells (n=3, mean+SD); where non-targeted SC-NPs were used as controls; and where MFI: mean fluorescence intensity. FIG. 26 shows data plots depicting the confirmation of cytotoxicity using a lactate dehydrogenase assay, i.e., dose-dependent cytotoxicity of αmCherry-[DTX]NPs against HEK293T-mCherry cells (a), αEGFR-[DTX]NPs against SKOV3 cells (b), and αHER2-[DTX]NPs against SKOV3 cells (c) measured at 72 h after 15 min of co-incubation (n=3, mean±SD); free DTX, non-targeted SC-[DTX]NPs, and the respective unloaded nanoparticles (αmCherry-NPs, αEGFR-NPs, and αHER2-NPs) were used as controls. FIG. 27 shows images and a data plot demonstrating the effect of drug-loaded modularly functionalized nanoparticles on microtubule structure and mitotic arrest. Panel (a) of FIG. 27 shows fluorescent images depicting fluorescent visualization of HEK293T-mCherry cells after treatment with PBS, SC-[DTX]NPs, or αmCherry-[DTX]NPs; blue: nuclei (DAPI), green: α-tubulin, magenta: nanoparticle (DiD); scale bar: 20 μm. Panel (b) of FIG. 27 shows fluorescent images depicting fluorescent visualization of SKOV3 cells (EGFR+ and HER2+) after treatment with PBS, SC-[DTX]NPs, αEGFR-[DTX]NPs, and αHER2-[DTX]NPs; blue: nuclei (DAPI), green: a-tubulin, magenta: nanoparticle (DiD); scale bar: 20 μm. Panel (c) shows a data plot depicting the percentage of SKOV3 cells in the G2 phase of the cell cycle after treatment with SC-[DTX]NPs, αEGFR-[DTX]NPs, and αHER2-[DTX]NPs (n=3, mean+SD).

SC-NPs were incubated with ST-αmCherry, ST-αEGFR, and ST-αHER2 to generate the corresponding targeted nanoformulations (denoted αmCherry-NPs, αEGFR-NPs, and αHER2-NPs), followed by incubation with cells expressing the cognate receptors (FIG. 20). First, αmCherry-NPs were incubated with HEK293T-mCherry cells, and dose-dependent targeting was observed using flow cytometry (FIG. 19A and FIG. 21A). At the concentrations that were evaluated, αmCherry-NPs exhibited no interactions with wild-type HEK293T (FIG. 22A). Similarly, both αEGFR-NPs and αHER2-NPs were efficient at targeting SKOV3 cells positive for both EGFR and HER2, whereas SC-NPs showed very limited interaction with the cells (FIGS. 4B, 4C and FIGS. 21B, 21C). In contrast, αEGFR-NPs did not show any affinity towards MDA-MB-453 cells that were EGFR, and αHER2-NPs were unable to target MDA-MB-231 cells that were HER2-(FIGS. 22B and 22C). Targeting specificity was confirmed in co-culture assays where the modularly functionalized nanoparticles were incubated with a mixture of cells both positive and negative for their cognate receptor (FIG. 19D-19F and) . Each nanoformulation preferentially bound to the appropriate target cell as verified by both fluorescent imaging and flow cytometry. The binding of each functionalized nanoformulation was also evaluated in the presence of the corresponding free soluble ligand as a blocking agent (FIG. 19G-19I and) . In the absence of blocking, all targeted nanoparticles interacted efficiently with cells expressing the appropriate receptor, whereas binding was completely abrogated with blocking. It was confirmed that the nanoparticles could be internalized by the target cells after binding (FIG. 25).

To evaluate the potential of the modular CNP formulations for cancer treatment, docetaxel (DTX) was selected as a model chemotherapeutic. The drug was encapsulated into the self-assembled PLGA cores via hydrophobic interactions. The resulting DTX-loaded SC-NPs (denoted SC-[DTX]NPs) were then functionalized with ST-αmCherry, ST-αEGFR, or ST-αHER2 to yield the corresponding targeted nanoformulations (denoted αmCherry-[DTX]NPs, αEGFR-[DTX]NPs, or αHER2-[DTX]NPs). Each targeted nanoparticle was then incubated with cancer cells expressing the cognate receptor for 15 min, after which any unbound nanoparticles were washed away. Following another 3 days of incubation, the cytotoxicity of each formulation was measured (FIG. 19J-19L). αmCherry-[DTX]NPs were highly effective against HEK293T-mCherry cells, exhibiting a half maximal inhibitory concentration (IC₅₀) of 4.0 ng mL⁻¹. Similarly, both αEGFR-[DTX]NPs and αHER2-[DTX]NPs were potent against SKOV3 cells, displaying IC₅₀ values of 3.2 ng mL⁻¹ and 4.3 ng mL⁻¹, respectively. These example results were confirmed using an orthogonal assay quantifying cell death (FIG. 26). Notably, neither free DTX nor the non-targeted SC-[DTX]NP formulation was cytotoxic under the same experimental conditions, highlighting the strong targeting effect of the CNPs after modular functionalization. No toxicity was observed from any of the targeted nanoparticles without DTX. The activity of the DTX on the target cells was validated by fluorescently visualizing microtubule disruption, as well as by assessing mitotic arrest (FIG. 27).

### In Vivo Tumor Targeting, Treatment Efficacy, and Safety

The performance of the modularly functionalized CNPs was next evaluated in vivo using tumor xenografts.

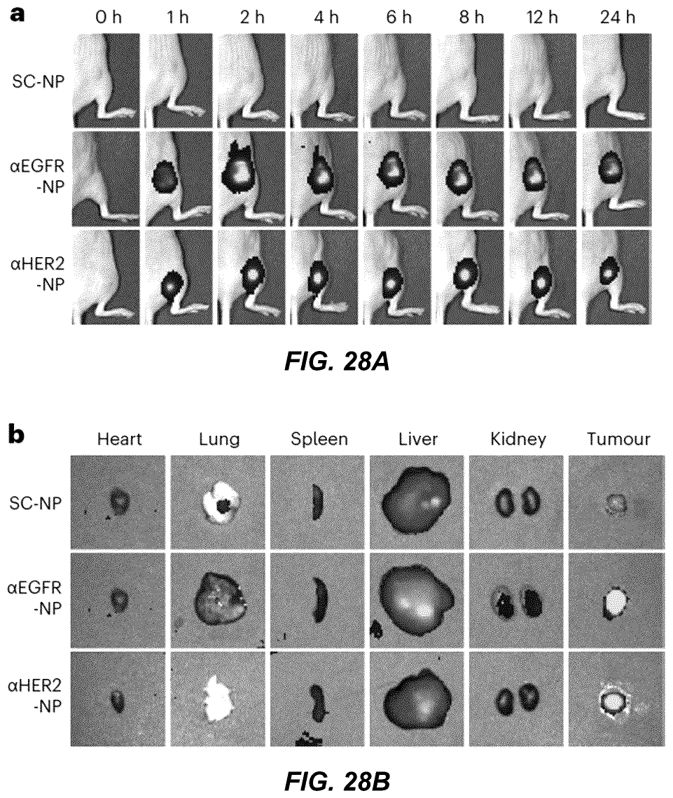

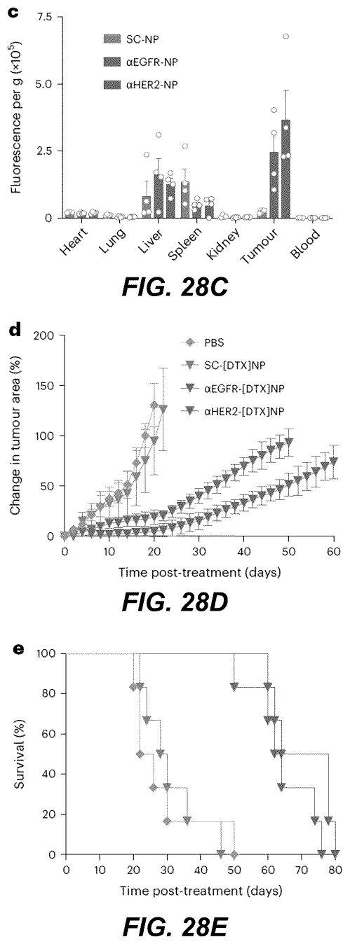

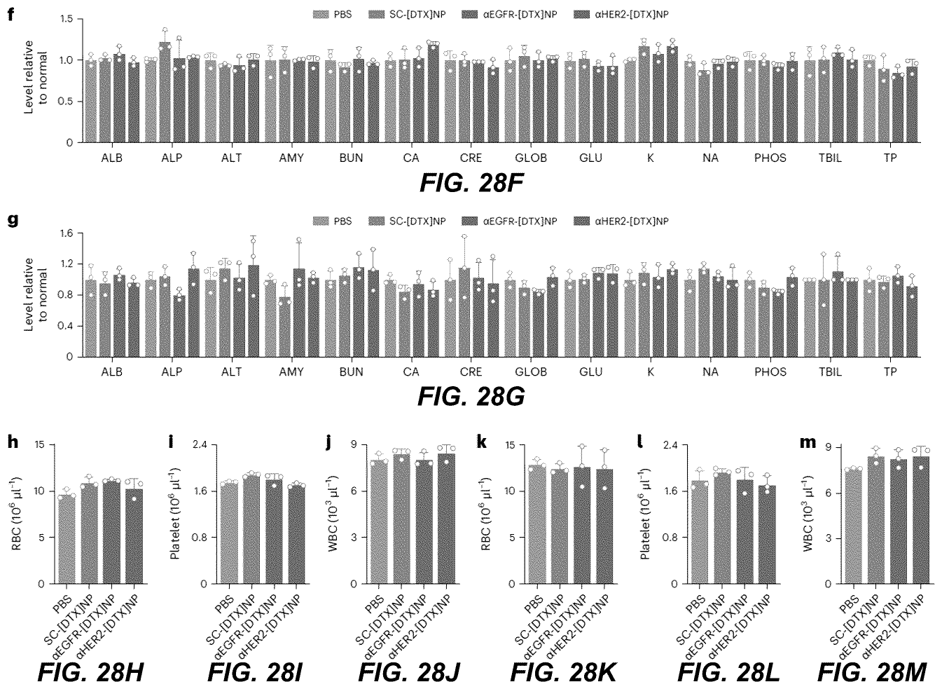

FIG. 28A-28M show images and data plots from example implementations demonstrating in vivo tumor targeting, therapeutic efficacy, and safety of the exemplary modularly functionalized nanoparticles, in accordance with the present technology. FIG. 28A shows an image panel depicting representative live fluorescent imaging of tumors at various timepoints after intravenous administration of SC-NPs, αEGFR-NPs, and αHER2-NPs. FIG. 28B shows an image panel depicting representative ex vivo fluorescent imaging of tumors and major organs collected 24 h after intravenous administration of SC-NPs, αEGFR-NPs, and αHER2-NPs. FIG. 28C shows a data plot depicting weight-normalized fluorescence of tumors and major organs collected 24 h after intravenous administration of SC-NPs, αEGFR-NPs, and αHER2-NPs (n=4 biological replicates, mean+SEM). FIG. 28D shows a data plot depicting growth kinetics of SKOV3 tumors treated intravenously with PBS, SC-[DTX]NPs, αEGFR-[DTX]NPs, and αHER2-[DTX]NPs (n=6 biological replicates, mean+SD). FIG. 28E shows a data plot depicting the survival of mice in FIG. 28D over time (n=6 biological replicates). FIGS. 28F and 28G show data plots depicting serum biochemistry of immunocompetent mice on day 1 (FIG. 28F) and day 10 (FIG. 28G) after intravenous administration of PBS, SC-[DTX]NPs, αEGFR-[DTX]NPs, and αHER2-[DTX]NPs; where injections were performed on days 0, 3, 6, and 9 (n=3 biological replicates, mean+SD); and where the following abbreviations are: ALB: albumin, ALP: alkaline phosphatase, ALT: alanine transaminase, AMY: amylase, BUN blood urea nitrogen, CA: calcium, CRE: creatinine, GLOB: globulin (calculated), GLU: glucose, K: potassium, NA: sodium, PHOS: phosphorus, TBIL: total bilirubin, TP: total protein. FIG. 28H-28M show data plots depicting red blood cell (RBC) (FIGS. 28H, 28K), platelet (FIGS. 281, 28L), and total white blood cell (WBC) (FIGS. 28J, 28M) counts of immunocompetent mice on day 1 (FIGS. 28H-28J) and day 10 (FIGS. 28K-28M) after intravenous administration with PBS, SC-[DTX]NPs, αEGFR-[DTX]NPs, and αHER2-[DTX]NPs; where injections were performed on days 0, 3, 6, and 9 (n=3 biological replicates, mean+SD).

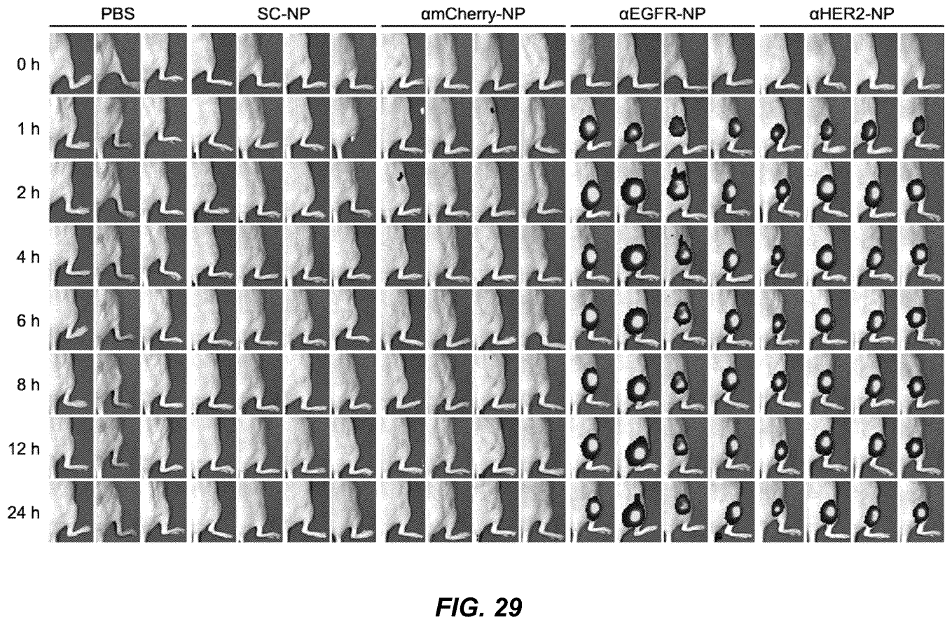

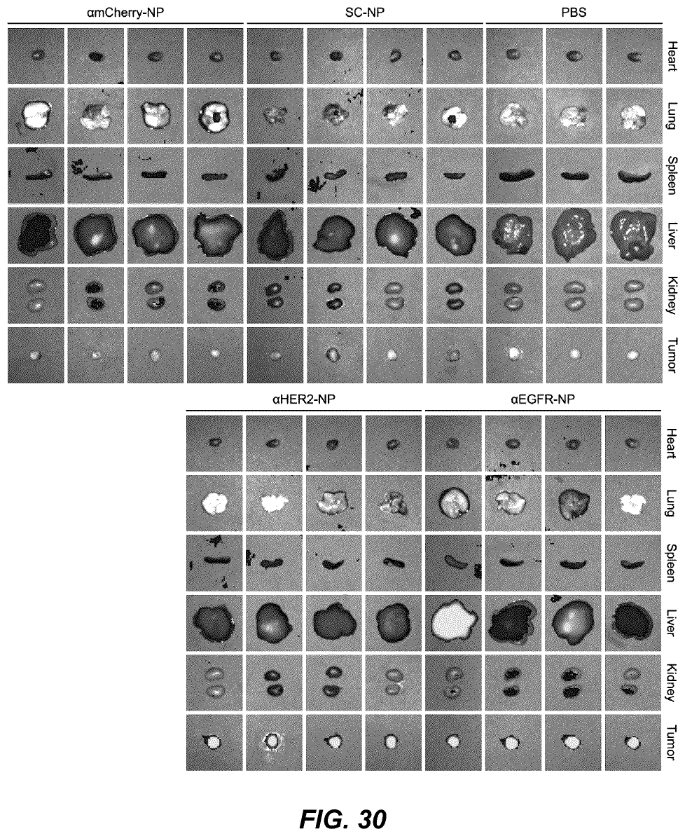

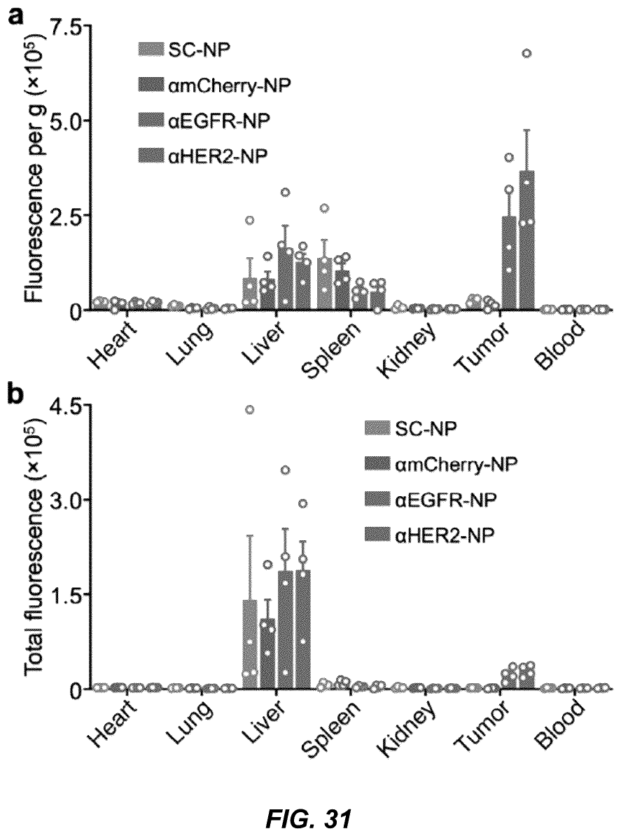

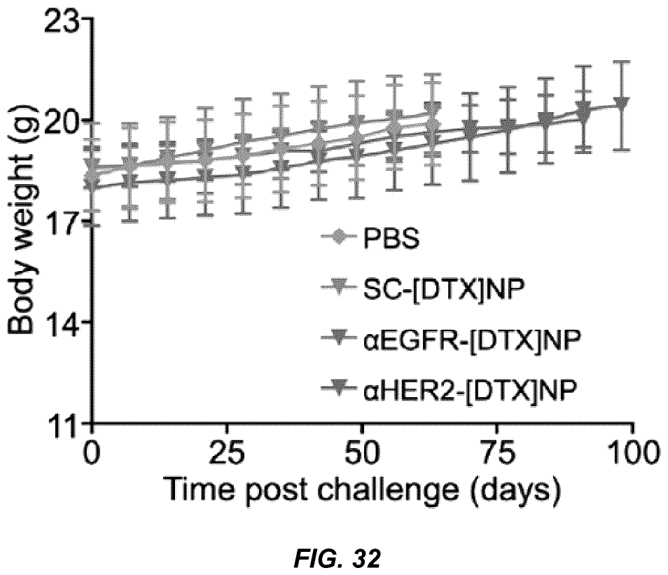

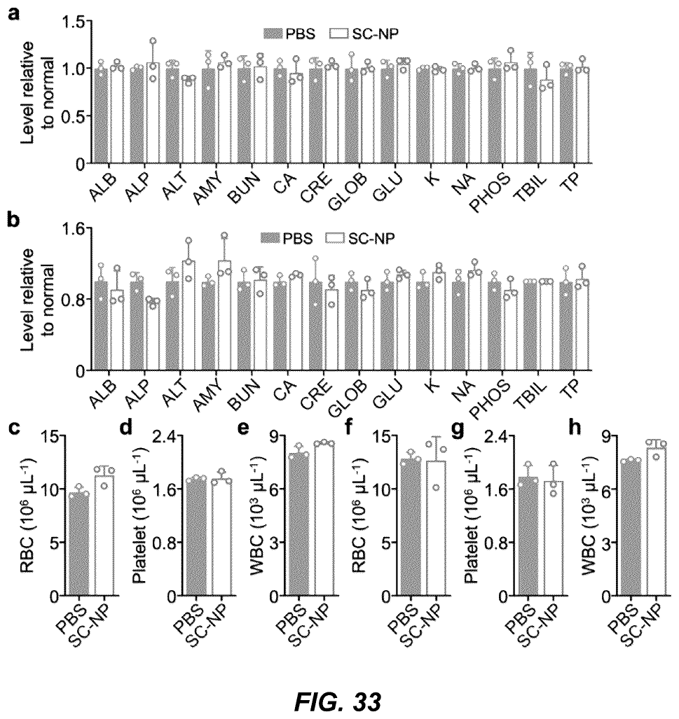

FIGS. 29-33 show images and data plots of example in vivo characterization results of modular functionalization on cell membrane-coated CNPs and target cell binding, in accordance with the present technology. FIG. 29 shows a panel of images showing in vivo tumor targeting, i.e., live fluorescent imaging of tumors at various timepoints after intravenous administration of SC-NPs, αmCherry-NPs, αEGFR-NPs, and αHER2-NPs. FIG. 30 shows a panel of images showing nanoparticle biodistribution in organs from an in vivo animal model, i.e., ex vivo fluorescent imaging of tumors and major organs collected 24 h after intravenous administration of SC-NPs, αmCherry-NPs, αEGFR-NPs, and αHER2-NPs. FIG. 31 shows data plots depicting the quantification of nanoparticle biodistribution, i.e., demonstrating the weight-normalized (a) and total fluorescence (b) of tumors and major organs collected 24 h after intravenous administration of SC-NPs, αmCherry-NPs, αEGFR-NPs, and αHER2-NPs (n=4, mean±SEM). FIG. 32 shows a data plot depicting results form body weight monitoring during therapeutic efficacy study, i.e., showing body weight over time of SKOV3 tumor-bearing mice treated intravenously with PBS, SC-[DTX]NPs, αEGFR-[DTX]NPs, and αHER2-[DTX]NPs (n=6, mean±SD). FIG. 33 shows data plots of example safety results of SC-NPs, where data plots (a) and (b) show serum biochemistry of immunocompetent mice on day 1 (a) and day 10 (b) after intravenous administration of PBS and SC-NPs, where injections were performed on days 0, 3, 6, and 9 (n=3, mean+SD), and where the abbreviations in FIG. 33 are: ALB: albumin, ALP: alkaline phosphatase, ALT: alanine transaminase, AMY: amylase, BUN blood urea nitrogen, CA: calcium, CRE: creatinine, GLOB: globulin (calculated), GLU: glucose, K: potassium, NA: sodium, PHOS: phosphorus, TBIL: total bilirubin, TP: total protein. Data plots (c)-(h) of FIG. 33 shows Red blood cell (RBC) (c, f), platelet (d, g), and white blood cell (WBC) (e, h) counts of immunocompetent mice on day 1 (panels (c)-(e)) and day 10 (panel (f)-(h)) after intravenous administration with PBS and SC-NPs; injections were performed on days 0, 3, 6, and 9 (n=3, mean+SD).

To establish the in vivo model, nude mice were subcutaneously implanted with SKOV3 cells. Fluorescently labeled αEGFR-NPs or αHER2-NPs were intravenously administered via the tail vein, and the animals were subjected to live imaging over 24 h (FIG. 28A and FIG. 29). For both targeted formulations, strong fluorescent signal was observed at the tumor site within 1 h, followed by a slight decay over time. In contrast, negligible targeting was observed for the non-targeted SC-NP formulation. The results were corroborated by ex vivo imaging and quantitative analysis of organ homogenates, where strong signal in the tumors was again observed only for mice receiving either αEGFR-NPs or αHER2-NPs (FIGS. 28B and 28C, and FIGS. 30 and 31). It was confirmed that nanoparticles functionalized with a mismatched targeting ligand did not show appreciable tumor accumulation (FIG. 31). On a per organ basis, the nanoparticles were predominantly found in the liver, as expected. Therapeutic efficacy was evaluated using DTX-loaded nanoformulations against the same SKOV3 model. Once tumor sizes reached an average of 70 mm², the nanoparticles were injected intravenously at a drug dosage of 3 mg kg⁻¹ every 3 days for a total of 4 administrations (FIGS. 28D and 28E). Both αEGFR-[DTX]NPs and αHER2-[DTX]NPs were able to significantly control tumor growth, whereas SC-[DTX]NPs had considerably less impact. Similar trends were observed when monitoring survival. While SC-[DTX]NPs were able to extend median survival from 24 days to 29 days after the start of treatment, αEGFR-[DTX]NPs and αHER2-[DTX]NPs further prolonged median survival to 63 days and 71 days, respectively.

Over the course of treatment, none of the drug-loaded nanoformulations had a significant impact on the body weight of the mice, suggesting a favorable safety profile and potential for dose sparing (FIG. 32). To further evaluate tolerability and potential off-target side effects, SC-NPs, SC-[DTX]NPs, αEGFR-[DTX]NPs, and αHER2-[DTX]NPs were administered into immunocompetent mice on the same schedule as the efficacy study, and samples were collected 1 and 10 days after the first injection for analysis (FIGS. 28F-28M and FIG. 33). In a blood chemistry panel, all parameters for mice receiving the drug-loaded nanoparticles were consistent with those for control mice. No differences were observed in blood counts, including for red blood cells, platelets, and white blood cells.

Example Materials and Method for the Example Implementations of Modular Functionalized CNPs with a SpyTag/SpyCatcher Ligand-Protein Binding System

Cell culture, engineering, and characterization. In the example implementations, all cells were maintained in Dulbecco's modified Eagle medium (DMEM; Corning) supplemented with 10% bovine calf serum (Hyclone) and 1% penicillin-streptomycin (Gibco). To generate the HEK293-SC cell line, a gene encoding for transferrin receptor (TfR)-anchored SpyCatcher003 fused to a myc-tag and sfGFP was isolated from pENTR4-TfR-sfGFP-myc tag-SpyCatcher003 (#133451, Addgene) by a polymerase chain reaction (PCR). The pQCXIH retroviral expression vector (Clontech) was linearized using the restriction enzymes BamHI-HF (New England BioLabs) and HindIII (New England BioLabs). The SpyCatcher003 genetic construct was then inserted into the pQCXIH vector using an In-Fusion HD cloning kit (TakaraBio). To generate HEK293T-mCherry cells, a gene encoding for mCherry fused with the transmembrane domain of platelet derived growth factor receptor β was synthesized using GeneArt (Invitrogen). The mCherry construct was inserted into a linearized pLenti-Puro-CMV lentiviral expression vector (Charles River Laboratories) using an In-Fusion HD cloning kit. Each constructed plasmid was transformed into DH5α Escherichia coli (New England BioLabs) for amplification according to the manufacturer's protocol. The completed SpyCatcher and mCherry plasmids were used to transfect AmphoPhoenix cells (National Gene Vector Biorepository) or HEK293T cells (CRL-3216, American Type Culture Collection), respectively. After 48 h, virus-containing cell culture supernatant was collected, followed by the addition of polybrene (Sigma-Aldrich) to the viral supernatant at a final concentration of 8 μg mL⁻¹. The mixtures were then incubated with wild-type HEK293 (CRL-1573, American Type Culture Collection) or HEK293T cells to produce HEK293-SC and HEK293T-mCherry, respectively. The viral transductions were facilitated by centrifuging the tissue culture plates at 800g for 90 min at 32° C. After 48 h, antibiotic selection was initiated using 500μg mL-1 of hygromycin B (InvivoGen) for HEK293-SC cells or 2 μg mL⁻¹ of puromycin (Gibco) for HEK293T-mCherry cells. Cells with high expression were sorted using a Becton Dickinson FACSAria Fusion flow cytometer. For HEK293-SC, a limiting dilution was performed for monoclonal selection. The final HEK293-SC and HEK293T-mCherry cell lines were maintained in media containing 300 μg mL⁻¹ of hygromycin B and 1 μg mL⁻¹ of puromycin, respectively.

SpyCatcher expression on HEK293-SC cells was confirmed by detecting the attached sfGFP, either by fluorescence microscopy or by flow cytometry using Alexa Fluor 647 anti-GFP (FM264G, BioLegend). mCherry expression was confirmed by directly detecting its fluorescence using flow cytometry. SKOV3 (HTB-77, American Type Culture Collection), MDA-MB-231 (CRM-HTB-26, American Type Culture Collection), and MDA-MB-453 (HTB-131, American Type Culture Collection) cells were evaluated for EGFR and HER2 expression by flow cytometry using Alexa Fluor 647 anti-human EGFR (AY13, BioLegend) and APC anti-human erbB2/HER2 (24D2, BioLegend), respectively. All data was collected using a Becton Dickinson LSR II flow cytometer, and analysis was performed using FlowJo software. Visualization was performed in a Nunc Lab-Tek II chamber slide (Thermo Scientific) using a Keyence BZ-X710 fluorescence microscope in VECTASHIELD antifade mounting medium with DAPI (Vector Laboratories).

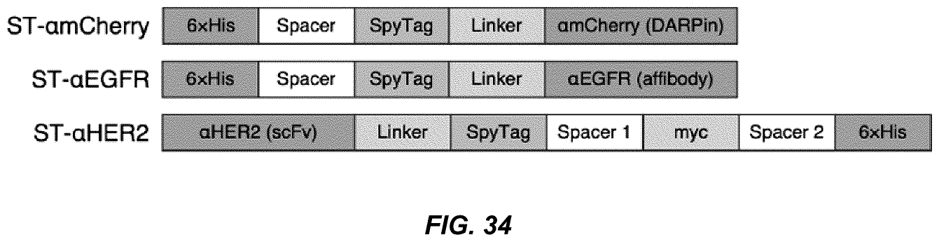

Production of SpyTag-labeled ligands. The plasmid for ST-mKate2 (#133452, Addgene) was transformed into BL21-CodonPlus (DE3)-RIPL competent cells (Agilent) using the manufacturer's protocol. To generate plasmids for ST-αmCherry and ST-αEGFR (FIG. 34), the genetic sequences for the DARPin-based αmCherry and affibody-based αEGFR were isolated respectively from pCASP-SptP120-3m160-HilA ( #153325, Addgene) and pET21-10XHis-GST-HRV-dHer1cys (#73218, Addgene) by PCR. These inserts were fused with the genetic sequence for SpyTag using NEBuilder HiFi DNA assembly (New England Biolabs).

FIG. 34 shows a schematic representation of the example engineered constructs of the example implementations. As shown in the schematic of FIG. 34, ST-αmCherry and ST-αEGFR includes a 6xHis tag, an amino acid spacer (SSGLVPRGSH), SpyTag, a glycine-serine linker (SGGGSG), and either an αmCherry DARPin or αEGFR affibody. As shown in the schematic of FIG. 34, ST-αHER2 is composed of an αHER2 scFv, a glycine-serine linker (GGGGS(SSSSG)6), SpyTag, an amino acid spacer (SGGGSGSRGGP), a myc tag, an amino acid spacer (NSAVD), and a 6xHis tag.

The expression plasmid pET28a (Sigma-Aldrich) was prepared using a double restriction digest with BamHI-HF and Ndel (New England BioLabs). The DNA inserts were then subcloned into the linearized pET28a using an In-Fusion HD cloning kit, followed by transformation into BL21-CodonPlus (DE3)-RIPL competent cells. Each construct contained a His-tag for downstream purification and detection purposes. For protein production, a single colony from a freshly streaked Luria-Bertani agar (Difco) plate of each transformed cell was used to inoculate 1 L of AIM LB broth base including trace elements (Formedium) supplemented with 100μg mL⁻¹ of kanamycin (Sigma-Aldrich). The cells were cultured at 30° C. for 24 h with shaking at 200 rpm, harvested, and lysed by probe sonication in 50 mM Tris-HCl at pH 7.5 (Invitrogen) and 300 mM NaCl (Fisher Scientific) containing a protease inhibitor cocktail (Sigma-Aldrich) using a Fisherbrand Model 120 Sonic Dismembrator. Cell lysates were centrifuged at 30,000 g for 25 min before the SpyTag-labeled ligands were purified from the supernatant using Ni-NTA agarose (Qiagen) using the manufacturer's standard procedures. An optimized wash buffer containing 30 mM imidazole (Sigma-Aldrich) was used for ST-mKate2 and ST-αEGFR, and 40 mM imidazole was used for ST-αmCherry. The plasmid for ST-αHER2 (FIG. 34) was generated by isolating the genetic sequence for the scFv-based αHER2 (#10794, Addgene) by PCR and fusing it to the genetic sequence for SpyTag using NEBuilder HiFi DNA assembly. The resulting DNA fragment was then inserted into the pSecTag2A plasmid (Invitrogen) with an In-Fusion HD cloning kit, followed by transformation into competent Dh5a E. coli. The construct contained a His-tag and myc-tag for downstream purification and/or detection purposes. Plasmid DNA was isolated from the bacteria cultured in Luria-Bertani broth (Difco) using a GenElute HP plasmid midiprep kit (Sigma-Aldrich), which was then used to transfect HEK293T cells with the help of Lipofectamine2000 (Invitrogen) according to the manufacturer's protocol. After 24 h, the transfected cells were switched to serum-free media (SFM4HEK293, Hyclone) and cultured for another 48 h. The supernatant was collected and purified using HisPur Ni-NTA resin (Thermo Scientific) under native conditions with 50 mM imidazole washes according to the manufacturer's protocol. Each eluted protein was buffer exchanged into water using an Amicon Ultra-15 centrifugal filter unit (Millipore) with the appropriate molecular weight cutoff (3 kDa for ST-αEGFR, 10 kDa for ST-αmCherry, 30 kDa for ST-mKate2 and ST-αHER2). The concentration of each product was measured using a Pierce Rapid Gold BCA protein assay kit (Thermo Scientific).

To confirm successful purification, untransformed bacteria and samples taken from throughout the production process were mixed with NuPAGE 4x LDS sample loading buffer (Invitrogen) and heated for 10 min at 70° C. Then, 20 L of each sample was loaded into 15-well Bolt 4 to 12% Bis-Tris mini protein gels (Invitrogen) and run at 165 V for 45 min in Bolt MOPS SDS running buffer (Invitrogen). Lysate, eluate, and purified samples were normalized to 1 mg mL-1, whereas flow-through and wash samples were used directly without dilution. SeeBlue Plus2 pre-stained protein standard (Invitrogen) was used as a ladder. Protein banding was visualized using Coomassie Blue (Expedeon) staining. For western blot analysis, proteins were transferred to a 0.45-μm nitrocellulose membrane (Pierce) in Bolt transfer buffer (Invitrogen) at 15 V for 30 min. The membranes were then stained with anti-6-His epitope tag (6-His, BioLegend), followed by horseradish peroxidase-conjugated anti-mouse IgG (Poly4053, BioLegend) as a secondary stain. Membranes were developed with ECL western blotting substrate (Pierce) and imaged using a Bio-Rad ChemiDoc MP imaging system.

SpyTag-labeled ligand binding and targeting. To evaluate the functionality of the SpyTag-labeled ligands, the solution for each ligand was adjusted to 1x PBS using a 20x PBS stock (Teknova), followed by addition to the appropriate target cells at varying concentrations on ice for 10 to 25 min. To confirm SpyTag function, each ligand was incubated with wild-type HEK293 or HEK293-SC cells. To confirm targeting functionality, ST-αmCherry was incubated with wild-type HEK293T or HEK293T-mCherry cells, ST-αEGFR was incubated with MDA-MB-453 or SKOV3 cells, and ST-αHER2 was incubated with MDA-MB-231 or SKOV3 cells. Using flow cytometry, mKate2 fluorescence was detected directly. ST-αmCherry and ST-αEGFR were detected by their attached His-tag using Alexa Fluor 647 anti-6-His epitope tag (6-His, BioLegend), and ST-αHER2 was detected by its attached myc-tag using Alexa Fluor 594 anti-c-myc (9E10, BioLegend). It was confirmed that anti-c-myc was unable to detect the myc-tag connecting SpyCatcher003 with sfGFP on the surface of HEK293-SC cells, likely due to steric hindrance. All data were collected using a Becton Dickinson LSR II flow cytometer, and analysis was performed using FlowJo software. To visualize ST-mKate2 binding to HEK293-SC, the ligand was added to the cells in a Nunc Lab-Tek II chamber slide at a concentration of 0.5 mg mL⁻¹ and allowed to incubate on ice for 15 min. After removing the supernatant, visualization was performed using a Keyence BZ-X710 fluorescence microscope in VECTASHIELD antifade mounting medium with DAPI.

To confirm targeting specificity in a heterogeneous population of cells, the SpyTag-labeled ligands were incubated with positive target cells and negative control cells co-cultured together. To visualize binding via fluorescent imaging, each ligand was added to co-cultures seeded overnight in Nunc Lab-Tek chambered coverglass (Thermo Scientific). Cells were blocked using 1% bovine serum albumin (BSA; Sigma-Aldrich) in PBS for 10 min on ice. ST-αmCherry was incubated with wild-type HEK293T and HEK293T-mCherry cells, ST-αEGFR was incubated with MDA-MB-453 and SKOV3 cells, and ST-αHER2 was incubated with MDA-MB-231 and SKOV3 cells. Each ligand was incubated with the cells for 15 to 20 min on ice in a solution of 1% BSA in PBS. Unbound SpyTag-labeled ligands were washed away with 1x PBS, and then detection was performed using the appropriate antibody incubated for 30 min on ice. ST-αmCherry was detected using FITC anti-6-His epitope tag (6-His, BioLegend), ST-αEGFR was detected using Alexa Fluor 647 anti-6-His epitope tag, and ST-αHER2 was detected using Alexa Fluor 488 anti-c-myc (9E10, BioLegend). Finally, the mCherry target protein was visualized directly, while EGFR was detected using Alexa Fluor 488 anti-human EGFR (AY13, BioLegend), and HER2 was detected using APC anti-erbB2/HER2. Cells were imaged in VECTASHIELD antifade mounting medium with DAPI using a Keyence BZ-X710 fluorescence microscope. For validation by flow cytometry, HEK293T-mCherry cells were detected directly, whereas SKOV3 cells were stained using 1,1′-dioctadecyl-3,3,3′,3′-tetramethylindodicarbocyanine, 4-chlorobenzenesulfonate salt (DiD; Invitrogen) at a final concentration of 5 μM for the EGFR targeting assay and 1,1′-dioctadecyl-3,3,3′,3′-tetramethylindocarbocyanine perchlorate (DiI; Invitrogen) at a final concentration of 5 μM for the HER2 targeting assay. The stained target cells and unstained negative control cells were mixed in equal concentrations, blocked in 1% BSA in PBS for 10 min on ice, and incubated with the appropriate SpyTag-labeled ligand in 1% BSA in PBS for 15 to 20 min on ice. ST-αmCherry and ST-αHER2 were detected using the same antibodies as above, while ST-αEGFR was detected using FITC anti-6-His epitope tag. All data were collected using a Becton Dickinson LSR II flow cytometer, and analysis was performed using FlowJo software.

Nanoparticle fabrication, functionalization, and characterization. HEK293-SC membrane was isolated. Briefly, HEK293-SC cells were harvested and stored at −20° C. in a 50:50 mixture of complete DMEM and HyCro crypreservation media (Hyclone) until membrane derivation. To derive the membrane, cells were first washed using 30 mM Tris-HCl at pH 7.0 (Quality Biological) with 75.9 mM sucrose (Sigma-Aldrich) and 225 mM D-mannitol (Sigma-Aldrich). After the addition of phosphatase and protease inhibitor cocktails (Sigma-Aldrich), the cells were mechanically disrupted using a Kinematica Polytron PT 10/35 probe homogenizer. The membrane was then isolated by a series of differential centrifugations and stored in 0.2 mM ethylenediaminetetraacetic acid (EDTA; USB Corporation) until further use. Polymeric nanoparticle cores were synthesized by precipitating 1 mL of 0.66 dL g⁻¹ carboxyl-terminated PLGA (LACTEL Absorbable Polymers) dissolved at 10 mg mL⁻¹ in acetone into 1 ml of UltraPure DNase/RNase-free distilled water (Invitrogen). The acetone was then removed by evaporation under a vacuum. The PLGA cores were then mixed with the cell membrane, followed by sonication in a Fisher Scientific FS30D bath sonicator for 2 min to form SC-NPs. Note that all nanoparticle concentrations were expressed in terms of PLGA polymer weight. To optimize coating efficiency, SC-NPs were fabricated at various membrane protein to PLGA core ratios. Size was measured by dynamic light scattering using a Malvern Zetasizer Lab Red Label before and after adjustment to 1x PBS using 20x PBS stock. To confirm membrane coverage, SC-NPs were fabricated at different membrane coating ratios using PLGA cores containing 5% biotinylated PLGA (BP-29254, BroadPharm). Size was measured by dynamic light scattering using a Malvern Zetasizer Lab Red Label before and after 1 h of incubation with 10μg mL⁻¹ streptavidin (Thermo Scientific).

To synthesize loaded nanoparticles, a similar procedure was followed, except 1 μL of 1 mg mL⁻¹ 3,3′-dioctadecyloxacarbocyanine perchlorate (DiO; Invitrogen) in acetone, 1 μL of DiD in acetone, 1 μuL of 1 mg mL⁻¹ 1,1′-dioctadecyl-3,3,3′, 3′-tetramethylindotricarbocyanine iodide (DiR; Invitrogen) in acetone, and/or 100 μL of 10 mg mL-1 DTX (MedChemExpress) in acetone was added to the organic phrase prior to precipitation into 1 mL of 1 mM Tris-HCl at pH 8.0 (Corning). The DTX loading into PLGA was measured using an Agilent 1220 Infinity II gradient liquid chromatography system equipped with a C18 analytical column (Brownlee) and determined to be 4 wt%. To fabricate mKate2-NPs, αmCherry-NPs, αEGFR-NPs, and αHER2-NPs, the corresponding soluble ligands were mixed with SC-NPs on ice for 30 min at final concentrations of 0.8 mg mL⁻¹ and 0.5 mg mL⁻¹, respectively. To remove excess ligands, the mixture was run through a column packed with Sepharose CL-4B (Sigma-Aldrich), and fractions positive for DiR signal, as measured using a TECAN Spark 20M multimode microplate reader, were collected together.

Membrane coating yield was measured by incubating 100 L of nanoparticle samples with 2 mL of Rapid Gold BCA working solution for 5 min at 37° C. The absorbance of the samples was measured using a TECAN Spark 20M plate reader. The size (diameter) and surface zeta potential of PLGA nanoparticles, wild-type HEK293 membrane vesicles, HEK293-SC membrane vesicles, WT-NPs, SC-NPs, and mKate2-NPs were measured by dynamic light scattering with a Malvern Zetasizer Lab Red Label. For TEM imaging, samples were first adsorbed onto a 400-mesh carbon film grid (Electron Microscopy Sciences) for 10 min. After washing with water 3 times for 2 min each, the grid was negatively stained with 1% (w/v) uranyl acetate (Electron Microscopy Sciences) for 15 s. Images were acquired with a JEOL 1200 EX II electron microscope. For immunogold TEM analysis, nanoparticle samples were first blocked with 1% BSA in water for 1 h, followed by incubation with a primary antibody stain in 1% BSA in water for 1 h. Anti-ATP1A1 (BS-9570R, Bioss) was used to detect the extracellular domain of Na⁺/K⁺-ATPase, anti-GFP (G10362, Invitrogen) was used to detect the SpyCatcher construct, and anti-6x His tag (RM146, Invitrogen) was used to detect ST-mKate2. After washing by centrifugation at 21,100g for 10 min, gold-conjugated anti-rabbit IgG (G7277, Sigma-Aldrich) in 1% BSA in water was incubated with the nanoparticles for 1 h as the secondary stain. The nanoparticles were washed again with water by centrifugation and adsorbed onto 400-mesh carbon film grid for 10 min. The grids were washed with water 6 times for 2 min each and imaged using a JEOL 1200 EX II electron microscope.

In order to confirm successful transfer of SpyCatcher onto the nanoparticles, cell lysate, cell membrane, and membrane-coated nanoparticle samples derived from wild-type HEK293 or HEK293-SC cells were normalized to 1 mg mL⁻¹ and analyzed by western blotting using anti-6-His epitope tag as the primary stain; the blots were developed on film in an ImageWorks Mini-Medical/90 Developer. SeeBlue Plus2 pre-stained protein standard was used as a ladder. To assess stability in solution, the nanoparticles were adjusted to 10% sucrose (Sigma-Aldrich) and kept at 4° C. with size measurements every other day. Binding of ST-mKate2 to SC-NPs or WT-NPs was first assessed by subjecting ST-mKate2, SC-NPs, mKate2-NPs (prior to removal of excess ligands), or a mixture of ST-mKate2 and WT-NPs to SEC using columns packed with Sepharose CL-4B. The fluorescence of mKate2 and DiR for each collected fraction was measured using a TECAN Spark 20M multimode microplate reader. Irreversible protein binding was assessed by western blotting analysis as described above. SC-NPs, the SpyTag-labeled ligands, and the modularly functionalized nanoparticles (prior to removal of excess ligands) were normalized to 1 mg mL-1. For the primary stain, anti-6-His epitope tag was used to detect ST-mKate2, anti-6x His tag (1B7G5, ProteinTech) was used to detect ST-αmCherry and ST-αEGFR, and anti-c-myc (9E10, BioLegend) was used to detect ST-αHER2.

In vitro targeting and cytotoxicity assays. The affinity of each targeted nanoformulation towards cells expressing its cognate receptor was evaluated in vitro. αmCherry-NPs were tested with HEK293T-mCherry as the positive cell line, and wild-type HEK293 cells served as the negative control; αEGFR-NPs and αHER2-NPs were both tested with SKOV3 as the positive cell line, while MDA-MB-453 and MDA-MB-231 cells served as the negative controls, respectively. To conduct the study, 1×10⁵ of each target cells were collected from culture by scraping and incubated with increasing concentrations of the corresponding targeted nanoformulations or SC-NPs as a negative control in 1% BSA in PBS for 15 to 20 min on ice. After washing away any unbound nanoparticles, the targeting effect was assessed using flow cytometry to quantify the DiR fluorescence associated with each cell. All data were collected using a Becton Dickinson LSR II flow cytometer, and analysis was performed using FlowJo software. For the blocking study, the target cells were preincubated with 1 mg mL⁻¹ of the corresponding soluble ligand prior to the addition of the nanoformulations. To confirm uptake, each formulation was incubated with the appropriate cells while rocking and treated with trypsin-EDTA (Gibco) to remove surface-bound nanoparticles prior to analysis.

To confirm targeting specificity in a heterogeneous population of cells, the modularly functionalized nanoparticles were incubated with positive target cells and negative control cells co-cultured together with rocking. To visualize binding via fluorescent imaging, each nanoparticle was added to co-cultures seeded overnight in Nunc Lab-Tek chambered coverglass. Following blocking with 1% BSA in PBS for 10 min on ice, DiO-labeled αmCherry-NP was incubated with wild-type HEK293T and HEK293T-mCherry cells, DiD-labeled αEGFR-NP was incubated with MDA-MB-453 and SKOV3 cells, and DiO-labeled αHER2-NP was incubated with MDA-MB-231 and SKOV3 cells. Each formulation was incubated with the cells for 15 to 20 min on ice in a solution of 1% BSA in PBS. Unbound nanoparticles were washed away with 1x PBS. The mCherry target protein was visualized directly, while EGFR was detected using Alexa Fluor 488 anti-human EGFR, and HER2 was detected using APC anti-erbB2/HER2. Cells were imaged in VECTASHIELD antifade mounting medium with DAPI using a Keyence BZ-X710 fluorescence microscope. For validation by flow cytometry, HEK293T-mCherry cells were detected directly, whereas SKOV3 cells were stained using DiO at a final concentration of 5 μM for the EGFR and HER2 targeting assays. The stained target cells and unstained negative control cells were mixed in equal concentrations, blocked in 1% BSA in PBS for 10 min on ice, and incubated with the appropriate dye-labeled nanoparticles in 1% BSA in PBS for 15 to 20 min on ice. αmCherry-NPs were labeled with DiO, αEGFR-NPs were labeled with Dil, and αHER2-NPs were labeled with DiD. All data were collected using a Becton Dickinson LSR II flow cytometer, and analysis was performed using FlowJo software.

To determine cytotoxicity, target cells were seeded into 96-well tissue culture plates at 6×10³ cells per well and allowed to adhere overnight. HEK293T-mCherry cells were used to evaluate αmCherry-[DTX]NPs, while SKOV3 cells were used to evaluate αEGFR-[DTX]NPs and αHER2-[DTX]NPs. The cells were incubated with increasing concentrations of the drug-loaded nanoformulations for 15 min with rocking at 4° C. Any unbound nanoparticles were then removed, and the cells were washed twice with 200 μL of PBS. Then, 200 μL of fresh media was added to each well, and the cells were cultured for 3 days before cell viability was determined using a CellTiter 96 AQueous One Solution cell proliferation assay (Promega) according to manufacturer's instructions. Free DTX, SC-[DTX]NPs, and unloaded αmCherry-NPs, αEGFR-NPs, or αHER2-NPs were used as controls. Untreated cells were used to determine 100% viability, and cells treated with 4 mg mL⁻¹ free DTX in 40% DMSO were used to determine 0% viability. The cytotoxicity results were confirmed using a lactate dehydrogenase assay (BioLegend). Cells were treated as described above. After 3 days of culture in fresh media, cell death was determined using untreated cells as a 100% viability control and cells lysed with the provided buffer as a 0% viability control.

DTX activity was validated by visualizing changes in microtubule structure and performing cell cycle analysis. First, PBS, SC-[DTX]NPs, αmCherry-[DTX]NPs, αEGFR-[DTX]NPs, and αHER2-[DTX]NPs were incubated with the appropriate target cells for 15 min at 4° C. with rocking. After fixation with 10% phosphate-buffered formalin (Fisher Scientific) for 15 min on ice, the cells were permeabilized by incubating in methanol on ice for 10 min. For microtubule visualization, detection was performed using Alexa Fluor 488 anti-α-tubulin (DM1A, Invitrogen). Cells were imaged in VECTASHIELD antifade mounting media with DAPI using a Nikon AIR Confocal microscope. For cell cycle analysis, FxCycle Violet stain (Invitrogen) was added to the cells after permeabilization. All data were collected using a Becton Dickinson Fortessa X-20 flow cytometer, and analysis was performed using FlowJo software.

Animal care. Male CD-1 mice (4 to 6 weeks) and female nu/nu (nude) mice (6 weeks) were purchased from Charles River Laboratories. All animals were housed in a facility at the University of California San Diego (UCSD) under federal, state, local, and National Institutes of Health (NIH) guidelines. All animal experiments were performed in accordance with NIH guidelines and approved by the UCSD Institutional Animal Care and Use Committee (IACUC).

In vivo biodistribution and tumor treatment efficacy. To generate the tumor model, 1×10⁶ SKOV3 cells in 50% Matrigel (Corning) were subcutaneously inoculated into the right flank of female nude mice and allowed to grow to a size of 70 mm2. For the biodistribution study, PBS, SC-NPs, αmCherry-NPs, αEGFR-NPs, or αHER2-NPs were intravenously administered, and the mice were imaged at 0, 1, 2, 4, 6, 8, 12, and 24 h using a Xenogen IVIS 200 system to detect DiR fluorescence. At 24 h, the mice were euthanized, and their tumors and major organs were excised for ex vivo imaging using a Xenogen IVIS 200 system. For quantification, the organs were homogenized using 2-mm zirconia beads (BioSpec) using a BioSpec Mini-BeadBeater-16. DiR fluorescence in the blood and homogenized organs was measured using a TECAN Spark 20M multimode microplate reader. For the tumor treatment study, PBS, SC-[DTX]NPs, αEGFR-[DTX]NPs, or αHER2-[DTX]NPs were intravenously administered to mice at a drug dosage of 3 mg kg⁻¹ every 3 days for a total of 4 treatments. Tumor area and body weight were measured over time. The experimental endpoint was predefined as either a single tumor dimension>18 mm or tumor area>200 mm².

In vivo safety studies. To evaluate safety, PBS, SC-NPs, SC-[DTX]NPs, αEGFR-[DTX]NPs, or αHER2-[DTX]NPs were intravenously administered into immunocompetent male CD-1 mice every 3 days at a drug dosage of 3 mg kg⁻¹ for a total of 4 administrations. At 24 h after the first injection and 24 h after the final injection, whole blood was collected into Microvette 100 potassium-EDTA blood collection tubes (Sarstedt) for cell counts. To evaluate serum chemistry, blood was collected without an anticoagulant and allowed to clot for 30 min at room temperature before centrifuging at 3000g to isolate the serum. All analyses, except that for creatinine levels, were performed by IDEXX Bioanalytics Services. Creatinine was assayed using a creatinine colorimetric assay kit (Cayman Chemicals).

### Summary of Example Implementations

In summary of the example results from the example implementations described above, it is shown that a scalable, modular, and effective CNP platform that can be readily modified with soluble ligands in a modular manner has been developed. For example, using the same base formulation, a variety of components can be attached onto the nanoparticle surface to introduce new functionalities that would otherwise be cumbersome to engineer by other approaches. These example implementations demonstrate the relatively rapid and adaptable nature of the CNP platform which provides flexibility through the lack of extensive post-processing steps and easily scalable for multiple applications. Further, this CNP platform is able to incorporate a massive variety of soluble targeting ligands without modification of the membrane-bound anchor protein or nanoparticle substrate. In the example implementations described above, a SpyCatcher-SpyTag binding pair was used, which spontaneously forms a covalent isopeptide bond upon coming into contact. To demonstrate these example embodiments of the disclosed technology, various classes of ligands, including a fluorescent protein and three different targeting moieties, were employed. Notably, the example modularly functionalized CNPs exhibited strong binding to cancer cells expressing the corresponding cognate receptor both in vitro and in vivo. This resulted in significant enhancement of the cytotoxic activity of a model chemotherapeutic payload, enabling improved control of tumor growth and a prolongment of survival in a murine model.

The disclosed modular functionalized CNPs are envisioned to meet the increased demand for effective strategies to rapidly custom-tailor their functionality in a scalable manner. The approach presented here builds upon advances in genetic engineering of CNPs. Conventionally, genetic engineering to anchor a soluble protein to a substrate requires considerable investment of time and resources with each new formulation, regardless of previous work on similar formulations. This limited the functionality of cell membrane-coated nanoparticles as engineering desirable soluble ligands in a membrane-bound form was time-intensive and cost-prohibitive. Yet now, the disclosed technology provides a modular functionalization approach to streamline the process and develop an adaptable, modular platform for producing functionalized cell membrane-coated nanoparticle systems targeted for the desired application. For instance, it is envisioned that a large library of modular components may be developed, e.g., enabling SpyCatcher-expressing CNPs from different cell membrane sources (e.g., macrophages, cancer cells, stem cells, etc.) and various functional ligands to be readily combined depending on specific needs. As an example, the disclosed technology can enable a large library of particular surface-bound anchor-modified cells, such as SpyCatcher-engineered cells, SnoopCatcher-engineered cells, and DogCatcher-engineered cells, or other genetically-engineered cells with an cell membrane-expressed anchor for a molecular binding complex disclosed herein, where each has their own unique native functionalities, as a way to formulate a wide range of modular functionalized nanoparticles in accordance with the present technology. As shown by the example implementations, for example, use of the SpyCatcher system can also be advantageous compared with other types of conjugation approaches for CNP functionalization, since the anchor is inherently expressed by the engineered cell. As such, SC-NPs do not have to go through additional processing or purification prior to attachment of a SpyTag-labeled ligand, after which scalable processes such as tangential flow filtration could be used to remove any unreacted components.

While the example implementations discussed above focused on cancer treatment, the disclosed technology is readily applicable to targeting cells associated with other disease conditions or therapeutic modalities. It is noted that the disclosed modular functionalized cell membrane-coated nanoparticles can employ other binding systems and nanostructure configurations. For example, cells can be engineered to express orthogonal functionalization binding pairs, such as SpyCatcher-SpyTag binding pairs, SnoopCatcher/SnoopTag binding pairs, and/or DogCatcher/DogTap binding pairs, such that multiple functionalities can be introduced at the same time, i.e., on the same base cellular nanoparticle. Suitable genetically-encoded binding pairs could allow for expression of multiple unique membrane-bound anchor proteins in the cellular membrane which could form irreversible isopeptide bonds with their corresponding unique soluble ligand, allowing for a modular, multi-functionalized, and rapidly scalable nanoparticle platform. Also, for example, while PLGA nanoparticles can be used for nanomedicine applications and were utilized in the example implementations described above to validate the modularity of the disclosed technology, various types of materials and cores of different sizes can also be employed. As CNP formulations are developed using the disclosed modular approach, there are some key factors that need to be considered. For example, for prolonged applications, the immune response generated against the cell membrane coatings and any non-native ligands will need to be considered. To address this challenge, as such, the disclosed modular functionalization NP platform is designed to carefully select soluble ligands that are lowly immunogenic and employ cell lines in which membrane markers such as major histocompatibility complexes are downregulated. Other factors such as the impact of the coating process on membrane integrity and the generalizability of the approach to ligands with different size, net charge, and hydrophobicity characteristics are considered by the disclosed modular functionalization NP platform. Overall, the disclosed modular functionalization NP platform is envisioned to be a next-generation platform with enhanced utility across a wide range of biomedical applications.

### Example Embodiments and Implementations of Modular Functionalized Cellular Nanodiscs

Some embodiments of the disclosed modular functionalized cell membrane-coated nanostructure technology include modular functionalized cellular nanodiscs that can be used in a variety of applications, including for targeted delivery of chemotherapeutics into tumors. Variations of the example embodiments and example implementations of the modular functionalized cellular nanodiscs are described below.

The effective delivery of chemotherapeutic drugs to tumor sites is critical for cancer treatment and remains a significant challenge. The advent of nanomedicine has provided additional avenues for altering the in vivo distribution of drug payloads and increasing tumor localization. More recently, cell-derived nanoparticles, with their biocompatibility and unique biointerfacing properties, have demonstrated considerable utility for drug delivery applications. Here, it was demonstrated that cell membrane-derived nanodiscs can be employed for tumor-targeted delivery. To bestow active targeting capabilities to the cellular nanodiscs, an example embodiment of the modular functionalization cell membrane nanostructure strategy was utilized using an exemplary SpyCatcher system. This enables the nanodiscs to be covalently modified with any targeting ligand labeled with a short SpyTag peptide sequence. As a proof-of-concept, a model chemotherapeutic doxorubicin is loaded into nanodiscs functionalized with an affibody targeting epidermal growth factor receptor. The resulting nanoformulation demonstrates strong tumor targeting both in vitro and in vivo, and it is able to significantly inhibit tumor growth in a murine breast cancer model.

Cancer is recognized as the second leading cause of death worldwide and presents a significant public health challenge. While newer treatment modalities such as targeted therapies, immunotherapies, and various combination approaches have proven to be effective against certain cancers, chemotherapy remains a common frontline treatment. In general, chemotherapeutics function by targeting rapidly dividing cancer cells, whether by affecting cell division, damaging DNA, or inducing apoptosis. Despite their effectiveness, such therapies are oftentimes encumbered by off-target toxicity, as their mechanisms of action are not specific to cancer cells. This considerably limits amount of drug that can be administered to patients, providing an avenue for tumors to develop drug resistance mechanisms that ultimately result in recurrence. For chemotherapies, their dose-limiting toxicities are largely an issue of nonspecific delivery, as systemic administration results in the unwanted exposure of healthy tissue to cytotoxic drugs. Further, it is well-known that many potent cancer therapeutics suffer from low bioavailability due to their hydrophobic nature. As such, drug delivery strategies that can localize large amounts of payload more specifically into tumor sites have been highly sought after.

The emergence of nanotechnology has helped to transform the field of drug delivery, enabling more precise control over the in vivo distribution of pharmaceuticals. This is facilitated by the unique properties of nanoparticles, including their small size, large surface area-to-volume ratio, ability to encapsulate various drug payloads, and amenability to surface modification. Currently, several nanodrugs have been approved for clinical use against cancers, including Doxil, a doxorubicin-loaded liposome, and Abraxane, a paclitaxel-bound albumin nanoparticle. In general, first generation nanomedicine platforms have relied on passive targeting mechanisms such as the enhanced permeability and retention effect to accumulate at the tumor site. To further improve efficiency, later generation nanodrugs have employed active targeting, which can be achieved through the use of ligands such as antibodies, peptides, aptamers, and nutrient mimetics. More recently, biomimetic nanoparticles have been gaining attention due to their ability to recapitulate functions commonly found in nature. Among these, cell membrane-coated nanoparticles (CNPs), composed of a nanosized core and a cell membrane shell, have emerged as a promising platform. As a result of their cell-mimicking properties, CNPs are capable of immune evasion, inflammation targeting, and cancer homing, making them valuable tools for tumor targeting.

Building upon the CNP concept, it is possible to fabricate cellular nanodiscs (CNDs) composed of cell membrane stabilized by an amphiphilic polymer. CNDs, characterized by their extremely small size and disc-shaped morphology, have emerged as a promising nanoplatform for various biomedical applications. For example, they can excel at antigen delivery and have been employed to elicit potent antitumor and antibacterial immunity. CNDs have also been utilized to successfully neutralize various bacterial toxins both in vitro and in vivo.

Example embodiments and implementations of the utility of CNDs as a broadly applicable platform for cancer drug delivery are disclosed. To achieve this, a modular strategy that allows the rapid generation of different targeted nanoformulations is leveraged. Cell membrane is genetically engineered to express SpyCatcher, enabling it to be rapidly and irreversibly modified with any targeting ligand fused to a short SpyTag peptide sequence. Using this membrane, tumor-targeting CNDs loaded with doxorubicin (DOX), a model chemotherapeutic, were successfully generated. Overall, the formulation exhibits elevated localization to tumors expressing the targeted receptor, resulting in strong antitumor efficacy in a murine breast cancer model.

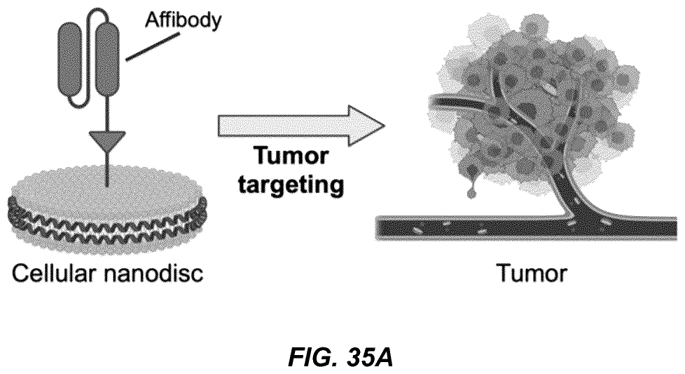

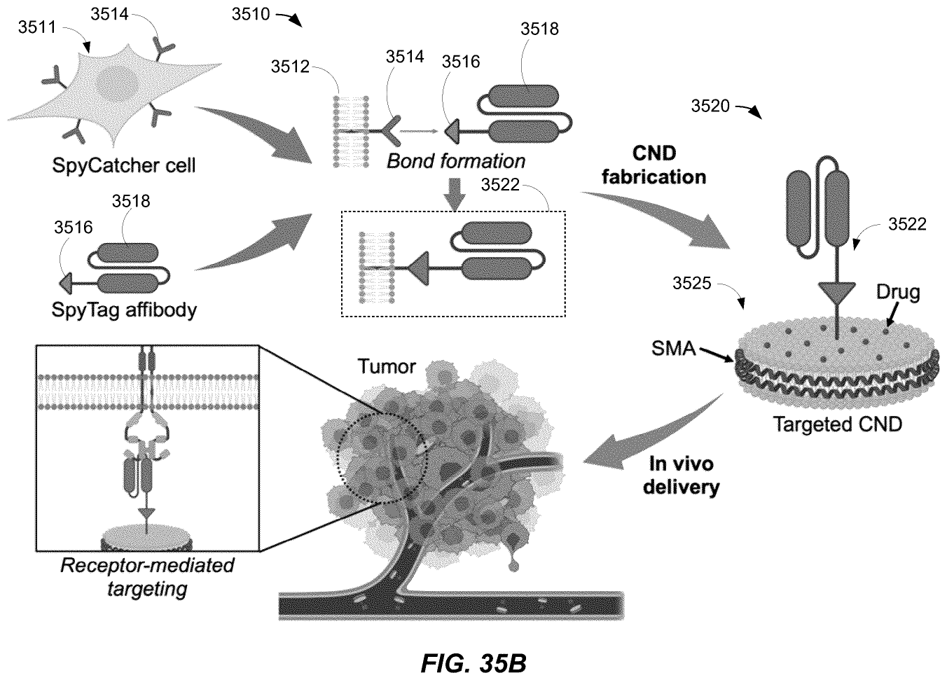

FIG. 35A shows a diagram depicting an example embodiment of a modular functionalized of cellular nanostructure for targeted tumor delivery, in accordance with the present technology.

FIG. 35B shows a diagram depicting an example embodiment of a method for modular functionalization of cellular nanodisc (CND) for targeted tumor delivery, in accordance with the present technology. The method includes a process 3510 where source cells 3511 are modified to express a binding protein 3514 (e.g., SpyCatcher) on their surface, which enables their cell membrane 3512 to be covalently functionalized with a targeting ligand 3518 fused to a binding ligand 3516 (e.g., the SpyTag peptide) that has binding affinity to the binding protein 3514. The method includes a process 3520 where the modified cell membrane 3522 (i.e., cell membrane 3512 with integrated binding proteins 3514 dispersed therein that binds the binding ligand 3516 that is covalently linked to the targeting ligand 3518) from the cells 3511 are synthesized with nanostructures (e.g., nanodiscs) to fabricate a modular functionalized cell membrane coated nanoparticle 3525, which, in some embodiments, can include a payload (e.g., drug). In FIG. 35B, the example modular, payload-loaded functionalized cell membrane coated nanoparticle 3525 includes a drug-loaded modular functionalized CND, e.g., which is fabricated with the help of a styrene-maleic acid (SMA) copolymer.

In some aspects in accordance with the present technology, a method of use of the modular, payload-loaded functionalized cell membrane coated nanoparticle 3525, e.g., a targeted modular functionalized CND, can include providing targeted modular functionalized CNDs in a living organism (e.g., patient) to target tumors positive for the cognate receptor of the affibody.

### Example Materials and Methods of Example Implementations

### Example Cell Culture and Protein Production Techniques

The MDA-MB-231 cell line was acquired from the American Type Culture Collection (CRM-HTB-26) and cultured in Dulbecco's modified Eagle medium (DMEM; Corning) supplemented with 10% fetal bovine serum (Gibco) and penicillin-streptomycin (Gibco). The SpyCatcher-expressing HEK293 cell line (denoted scCell) was constructed and cultured in complete DMEM supplemented with 300 μg/mL of hygromycin B (Invivogen).

An affibody targeting epidermal growth factor receptor (EGFR) was genetically fused to the SpyTag003 peptide and subcloned into an expression plasmid. To produce the resulting ligand (denoted STα), the plasmid was transformed into BL21-CodonPlus (DE3)-RIPL competent cells (Agilent) and plated onto Luria-Bertani (LB) agar (Difco). A single colony was picked and cultured in 1 L of auto-induction media LB broth base including trace elements (Formedium) with 100μg/mL kanamycin (Sigma-Aldrich) at 30° C. with shaking at 200 rpm. After 24 h, the bacteria were collected, resuspended in a buffer containing 300 mM NaCl (Fisher Scientific), 50 mM Tris-HCl (pH 7.5, Invitrogen), and a protease inhibitor cocktail (Sigma-Aldrich), and lysed using a Fisherbrand Model 120 Sonic Dismembrator. The cell lysate was then centrifuged at 30,000 g for 25 min, and the supernatant was collected to purify out the STα via an attached His-tag using nickel-nitrilotriacetic acid agarose (Qiagen) according to the manufacturer's procedure.

### Example CND Preparation

The cell membrane of scCell (denoted scMem) was derived. Briefly, the cells were washed 3 times with a buffer including 225 mM D-mannitol (Sigma-Aldrich), 76 mM sucrose (Sigma-Aldrich), and 30 mM Tris-HCl (pH 7.5, Quality Biological). After being resuspended in the same buffer supplemented with a protease inhibitor cocktail and a phosphatase inhibitor cocktail, the cells were disrupted using a Kinematica Polytron PT 10/35 probe homogenizer at 70% power for 20 passes. The cell homogenate was then centrifuged at 10,000 g for 25 min. The resulting supernatant was collected and centrifuged twice at 150,000 g for 35 min using a Beckman Coulter Optima XPN-80 ultracentrifuge. The pellet containing membrane material was resuspended in 0.2 mM ethylenediaminetetraacetic acid (EDTA; Invitrogen) in water and stored at −80° C. for further use.

To prepare affibody-functionalized and drug-loaded CNDs (denoted αND[DOX]), scMem was resuspended in water and incubated with STα at the appropriate weight ratios to form affibody-conjugated membrane (denoted αMem); the final membrane protein concentration was fixed at 1 mg/mL. The mixture was then vortexed at 300 rpm using a Fisher Scientific digital vortex mixer at 4° C. for 1 h. Doxorubicin hydrochloride (DOX; TCI Chemicals) was dissolved in water to create a 10 mg/mL stock solution, which was then added to αMem at a DOX to membrane protein weight ratio of 1:2, and the solution was sonicated for 5 s at 70% amplitude using a Fisher Scientific 150E Digital Sonic Dismembrator. An equal volume of styrene-maleic acid (SMA; SMALP 200, Cube Biotech) at 50 mg/mL was then added, and the solution was vortexed again at 300 rpm at 4° C. for 16 h. Non-solubilized lipid, protein, and free DOX were pelleted after ultracentrifugation at 150,000 g for 30 min. The CNDs in the supernatant were further purified and concentrated using Amicon Ultra centrifugal filters (30 kDa MWCO, Millipore Sigma).

CNDs labeled with the fluorescent dye Alexa Fluor 647 were prepared. For non-functionalized CNDs with or without DOX (denoted scND[DOX] or scND, respectively), as well as targeted CNDs without DOX (denoted αND), the same process was applied replacing STα and/or DOX with the same volume of buffer.

### Example Confirmation Techniques of Affibody Conjugation

To determine the dose-dependent conjugation of STα, 1 mg/mL of scMem was mixed with STα at different weight ratios and vortexed at 300 rpm at 4° C. for 1 h. The mixtures were then prepared in NuPAGE LDS sample buffer (Invitrogen). The samples were loaded into a Bolt 4-12% Bis-Tris 15-well mini protein gel (Invitrogen) and run at 165 V for 45 min in Bolt MOPS SDS running buffer (Invitrogen). Proteins in the gel were then transferred onto a 0.45-μm nitrocellulose membrane (Thermo Scientific) in Bolt transfer buffer (Invitrogen) at 15 V for 30 min. After blocking with 1% bovine serum albumin (BSA; Sigma-Aldrich) and 5% nonfat milk (Apex Bioresearch) in phosphate buffered saline (PBS; Corning) containing 0.05% (v/v) Tween 20 (National Scientific), the blots were immunostained with anti-6-His epitope tag (6-His, BioLegend) as the primary stain, followed by horseradish-peroxidase-conjugated anti-mouse IgG (Poly4053, BioLegend) as the secondary stain. Membranes were developed with ECL western blotting substrate (Pierce) and imaged using a Bio-Rad ChemiDoc MP imaging system. Free scMem and STα were used as controls.

### Example Characterization Techniques of CNDs

For size and zeta potential measurements, samples were resuspended in PBS and analyzed using a Malvern Zetasizer Lab Red Label. To assess stability, the nanoparticles were stored at 4° C., and their sizes were measured every 2 days for a total of 10 days. To visualize morphology, the samples were deposited onto a 400-mesh carbon film grid (Electron Microscopy Sciences), negatively stained with 1% uranyl acetate (Electron Microscopy Sciences), and then imaged using a JEOL JEM-1400Plus transmission electron microscope. The presence of STα in αMem and αND was confirmed by western blotting as described above. To determine drug encapsulation efficiency, scND[DOX] and αND[DOX] were disrupted in 80% (v/v) dimethyl sulfoxide (DMSO; Fisher Scientific). The fluorescent signal of DOX (excitation/emission =485/595 nm) was measured using a TECAN Spark 20M microplate multimode reader. To measure the release kinetics of DOX, 200 μL of scND[DOX] or αND[DOX] at a concentration of 1 mg/mL in PBS was added into Slide-A-Lyzer MINI dialysis devices (10K MWCO, Thermo Scientific) and floated on top of 300 mL of PBS at 37° C. At predetermined timepoints, 10 uL of sample was taken out for fluorescence measurement. Free DOX was used as a control.

### In Vitro Targeting

For imaging, 3×10⁵ MDA-MB-231 cells were seeded overnight into a 30 x 10 mm glass bottom tissue culture-treated dish (CELLTREAT). Dye-labeled scND or αND were added to the dish at a final membrane protein concentration of 5 μg/mL, followed by incubation for 2 h. For blocking, cells were preincubated with STα at a concentration of 1 mg/mL for 2 h. Imaging was performed using a Keyence BZ-X710 fluorescence microscope. For quantitative analysis, cells were seeded into a 24-well tissue culture plate (GenClone) at a density of 1×10⁵ per well. After overnight incubation, the cells were collected with 1 mM EDTA in PBS. For affibody blocking, cells were incubated with STα at a concentration of 1 mg/mL for 2 h. Afterwards, the cells were incubated with dye-labeled scND or αND at a membrane concentration of 5 g/mL for 2 h. Data was collected using a Becton Dickinson LSR-II flow cytometer, and analysis was performed using FlowJo software.

To explore the uptake kinetics of DOX, MDA-MB-231 cells were seeded into a 24-well tissue culture plate at a density of 1×105 per well, followed by incubation with free DOX, scND[DOX], and αND[DOX] at a drug concentration of 100 ng/ml. At predetermined timepoints, cells were collected with 1 mM EDTA in PBS, blocked with 1% BSA in PBS for 30 min at 4° C., and data was collected using a Becton Dickinson LSR-II flow cytometer. Data analysis was performed using FlowJo software. To visualize uptake, 3×10⁵ MDA-MB-231 cells were seeded overnight into a 30×10 mm glass bottom tissue culture-treated dish, followed by incubation with free DOX, scND[DOX], and αND[DOX] at a drug concentration of 100 ng/ml for 2 h. The cell nuclei were stained with Hoechst 33342 (Thermo Scientific) for 10 min prior to imaging, which was performed using a Keyence BZ-X710 fluorescence microscope.

### Example Cytotoxicity Assay

To evaluate cytotoxicity, 7.5×10³ MDA-MB-231 cells were seeded overnight into a 96-well tissue culture plate (GenClone). Cells were then treated with free DOX, scND[DOX], and αND[DOX] at increasing drug concentrations for 4 h, followed by replacement of the medium with fresh complete DMEM. After another 48 h of incubation, cells were washed once with PBS and incubated with 3-[4,5-dimethylthiazol-2-y1]-2,5-diphenyltetrazolium bromide (Sigma-Aldrich) for 1.5 h. Subsequently, the medium was removed, and 100 μL of DMSO was added to dissolve the formazan crystals. The absorbance at 570 nm was measured using a TECAN Spark 20M microplate multimode reader. Untreated cells were used as a 100% viability control, and those treated with 1% Triton X-100 (Sigma-Aldrich) were used as a 0% viability control.

### Animal Care in Example Implementations

Six-week-old male CD1 mice were obtained from Envigo. Six-week-old female nu/nu nude mice were obtained from Charles River Laboratories. All mice were housed in an animal facility at the University of California San Diego (UCSD) under federal, state, and local guidelines. All animal experiments were performed in accordance with National Institutes of Health guidelines and approved by the Institutional Animal Care and Use Committee (IACUC) of UCSD.

### Pharmacokinetics and Biodistribution

For the pharmacokinetics study, nu/nu mice were injected with 200 μL of dye-labeled scND and αND at a protein concentration of 0.66 mg/mL. At predetermined timepoints, a drop of blood was collected via submandibular puncture into microtubes coated with sodium heparin (Sigma-Aldrich). Then, 10 μL of each blood sample was diluted with 90 μL of PBS, and the fluorescent signal (excitation/emission=650/671 nm) of the diluted blood was measured using a TECAN Spark 20M microplate multimode reader. The percentage relative signal was calculated as (Fe-Fo)/(Fmax-Fo), where Fo represents the baseline fluorescence in untreated mice, Ft represents the fluorescence at time point t, and Fmax represents the fluorescence at 1 min.

To evaluate tumor targeting and biodistribution, nu/nu mice were inoculated intradermally with 5×10⁶ MDA-MB-231 cells in 50% Matrigel (Corning) on the left flank. Once the average tumor area reached ˜70 mm2, the mice were intravenously injected with 200 μL of dye-labeled scND and αND at a protein concentration of 0.66 mg/mL and imaged using a Xenogen IVIS 200 system at predetermined timepoints. After the final imaging at 24 h, the mice were euthanized, and their main organs and tumors were collected for ex vivo imaging using a Xenogen IVIS 200 system. To quantify the total fluorescence, tissue samples were homogenized with 2-mm zirconia beads (BioSpec) using a BioSpec Mini-BeadBeater-16, and the signals were measured using a TECAN Spark 20M microplate multimode reader.

### Example Implementation of an Antitumor Efficacy Study

The tumor model was constructed as described above. Once the average tumor area reached ˜35 mm², the mice were intravenously administered with free DOX, scND[DOX], or αND[DOX] at a drug concentration of 2.5 mg/kg via the tail vein every 3 days for a total of 4 injections. Tumor size and body weight were measured every other day. The experimental endpoint was predefined as tumor area>200 mm².

### Biosafety Study

CD1 mice were intravenously administered with free DOX, scND[DOX], or αND[DOX] at a drug concentration of 2.5 mg/kg. After 24 h, blood samples were collected via submandibular puncture and allowed to clot, after which the serum was derived by centrifugation at 3,000 g for 5 min. The samples were then sent to the UCSD Animal Care Program Diagnostic Services Laboratory for complete blood chemistry analysis.

### Example Results and Discussion of Example Implementations

Example Preparation of CNDs To fabricate the modular CNDs, wild-type HEK293 cells were first transduced to express a membrane-bound version of SpyCatcher003, which rapidly forms a covalent bond with the SpyTag003 peptide, and the resulting cell line was denoted scCell. An anti-epidermal growth factor receptor (EGFR) affibody was selected as a model targeting ligand and genetically fused with SpyTag. The modified affibody, denoted STα, was expressed in Escherichia coli and isolated using nickel-nitrilotriacetic acid affinity chromatography by leveraging its His-tag. Next, the plasma membrane from scCell (denoted scMem) was isolated and incubated with STα to form affibody-functionalized membrane (denoted αMem).

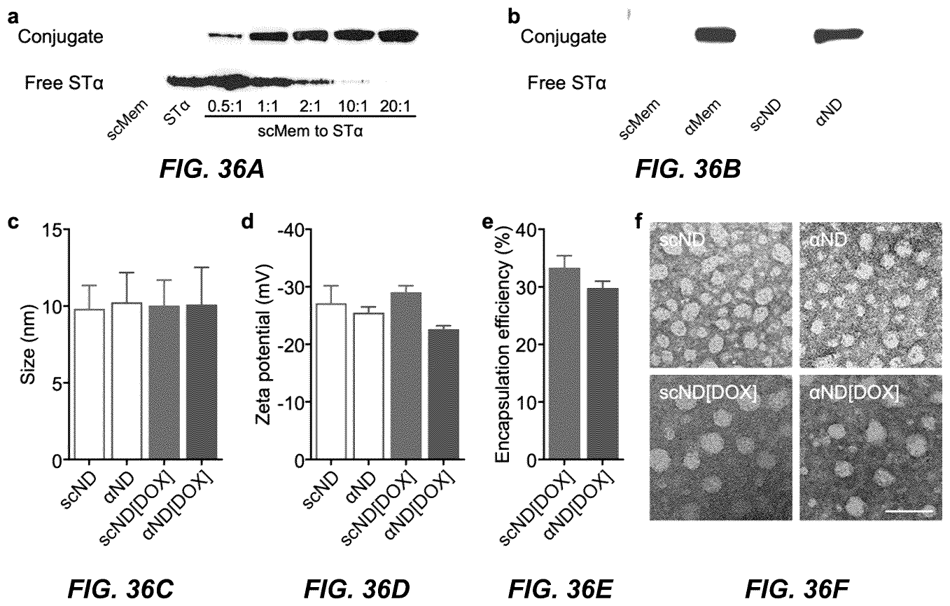

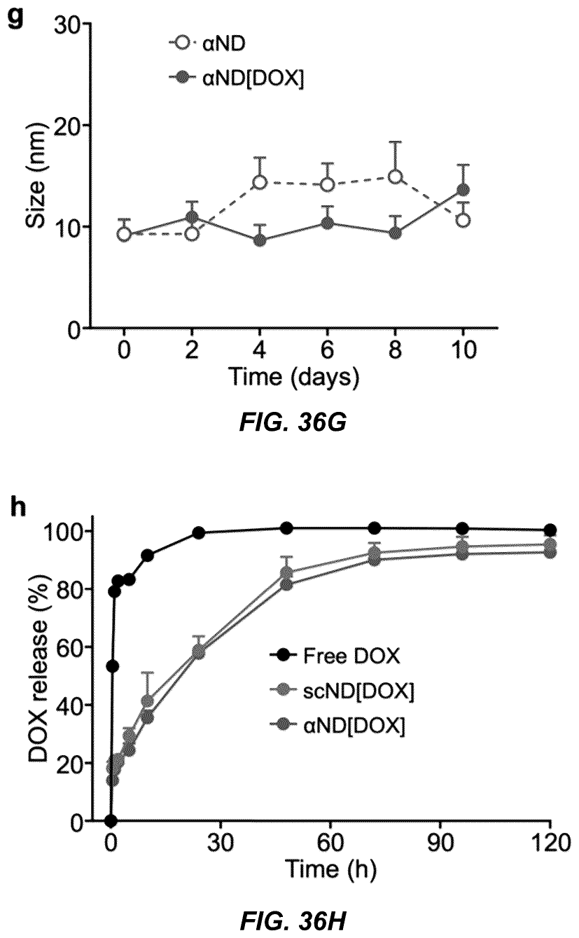

FIGS. 36A-36H show data images and data plots depicting an example fabrication implementation of example embodiments of CNDs and characterization of the fabricated CNDs. FIG. 36A shows western blot evaluating the conjugation of a SpyTag-labeled anti-EGFR affibody (STα) to SpyCatcher-expressing HEK293 cell membrane (scMem) at different protein weight ratios. FIG. 36B shows western blot probing for the presence of STα on scMem, affibody-functionalized membrane (αMem), nanodiscs fabricated from scMem (scND), and nanodiscs fabricated from ≢Mem (αND). FIGS. 36C and 36D show data plots showing size (FIG. 36C) and zeta potential (FIG. 36D) of scND, αND, DOX-loaded scND (scND[DOX]), and DOX-loaded αND (αND[DOX]) (n=3, mean+SD). FIG. 36E shows a data plot depicting encapsulation efficiency of DOX in scND[DOX] and αND[DOX] (n=3, mean+SD). FIG. 36F shows TEM images of scND, αND, scND[DOX], and αND[DOX] negatively stained with uranyl acetate; scale bar=50 nm. FIG. 36G shows a data plot depicting stability of αND and αND[DOX] in 1xX phosphate-buffered saline (PBS) at 4° C. (n=3, mean+SD). FIG. 36H shows a data plot depicting drug release kinetics for free DOX, scND[DOX], and αND[DOX] (n=3, mean+SD).

The irreversible binding between SpyCatcher and SpyTag was evaluated by incubating scMem with STα at various ratios (FIG. 36A). At a cell membrane to affibody protein weight ratio of approximately 10:1, nearly all STα was bound, and this ratio was selected for further experiments. CNDs were fabricated by incubating either scMem or αMem with a SMA copolymer. After fabrication, the successful functionalization of the final targeted CND formulation (denoted αND) was confirmed by western blotting (FIG. 36B). As expected, scNDs fabricated using scMem were not positive for STα. DOX was selected as a model anticancer drug for investigating the potential of the αND for nanodelivery. To encapsulate DOX within the final formulation, the drug was premixed with αMem, followed by the addition of SMA. Through the interaction between DOX and the cell membrane, the final drug-loaded and affibody-conjugated CND formulation (denoted αND[DOX]) was successfully formed. As a control, a corresponding non-functionalized drug-loaded formulation (denoted scND[DOX]) was generated.

### Characterization of the CND Formulations

The size of all formulations, including scND, αND, and their DOX-loaded counterparts, exhibited near-identical average sizes of approximately 10 nm when measured by dynamic light scattering (FIG. 36C). Their zeta potentials were also within a similar range, from around-22 to −27 mV (FIG. 36D). In terms of drug encapsulation efficiency, the drug incorporated into scND[DOX] and αND[DOX] equally well at approximately 30% (FIG. 36E). The morphology of both the empty and drug-loaded formulations was visualized using transmission electron microscopy (FIG. 36F). Consistent with previous reports, each exhibited a uniform circular shape, indicating that the physical form of the CNDs was minimally affected by the presence of DOX. Furthermore, when stored in 1x PBS solution at 4° C., the sizes of both αND and αND[DOX] were stable over a period of 10 days (FIG. 36G). Finally, the drug release from scND[DOX] and αND[DOX] was assessed in vitro, and it was revealed that approximately 60% of DOX was released within 24 h, and approximately 80% was released within 48 h, after which the kinetics plateaued (FIG. 36H).

### Example In Vitro Characterization

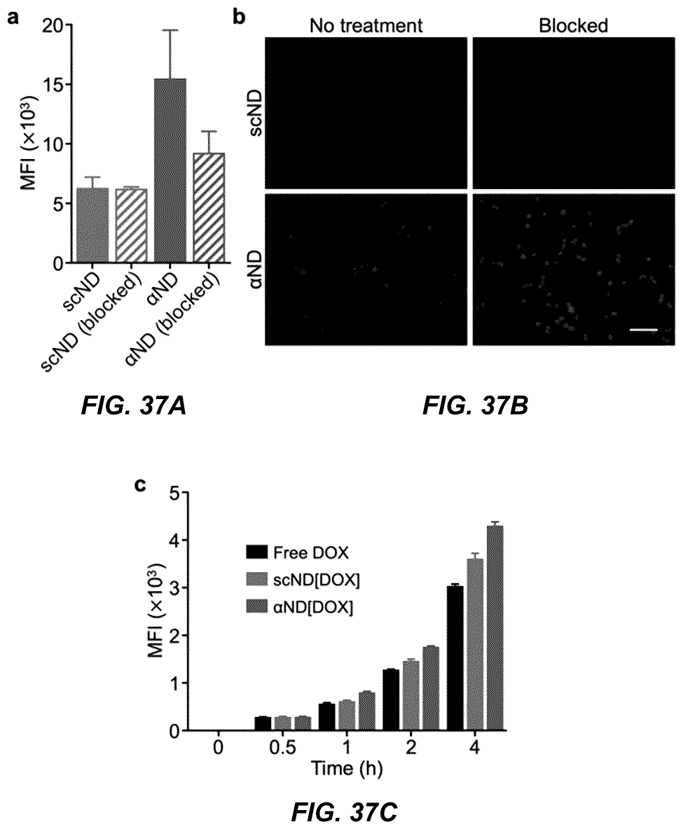

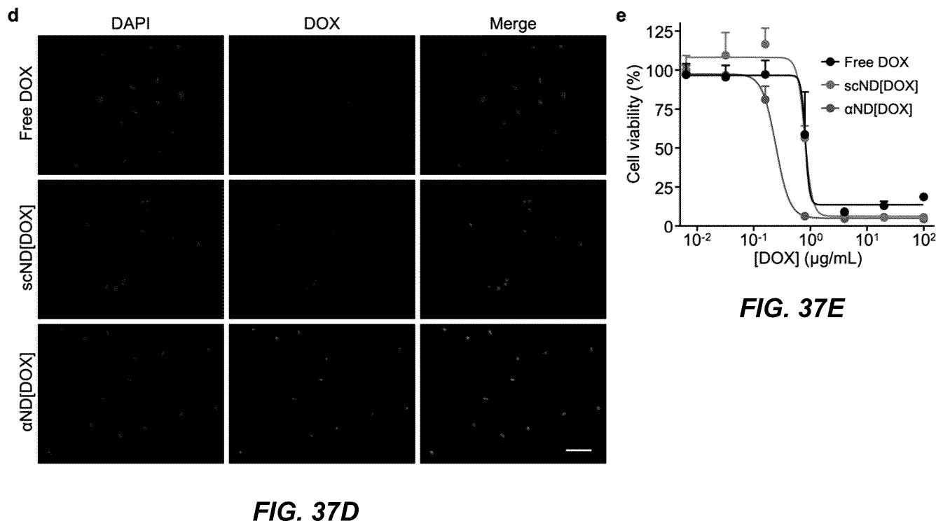

To evaluate if affibody conjugation could enhance the targeting ability of the CNDs, MDA-MB-231 cells positive for EGFR were incubated with dye-labeled scNDs or αNDs (FIGS. 37A-37B).

FIGS. 37A-37E show data images and data plots depicting an example in vitro characterization of example embodiments of CNDs. FIG. 37A shows a data plot depicting binding of dye-labeled scND and αND after 1 h of incubation with MD-MB-231 cells as measured by flow cytometry (n=3, mean+SD); free STα was used to block specific targeting. FIG. 37B shows representative images visualizing the binding of dye-labeled scND and αND to MD-MB-231 cells after 1 h of incubation; free STα was used to block specific targeting. MFI: mean fluorescence intensity; red: dye-labeled CND; scale bar=50 μm. FIG. 37C shows a data plot depicting time-dependent uptake of DOX delivered in free form, scND[DOX], and αND[DOX] by MDA-MB-231 cells (n=3, mean+SD). FIG. 37D shows representative images visualizing DOX uptake after 2 h of incubation in free form, scND[DOX], and αND[DOX]; blue: nuclei, red: DOX; scale bar=50 μm. FIG. 37E shows a data plot depicting a dose-dependent killing of MDA-MB-231 cells after 4 h of exposure to free DOX, scND[DOX], and αND[DOX], followed by another 48 h of incubation (n=6, mean+SD).

Compared to the scND group, the cells receiving αND showed significantly increased fluorescent signal when analyzing both by flow cytometry and by microscopy. The level of interaction was significantly reduced when the cells were pretreated with free STα as a blocking agent, confirming that the specific targeting ability of αND was mediated by the affibody functionalization. Next, it was evaluated if this enhanced targeting could improve the delivery of a drug payload in vitro. MDA-MB-231 cells were incubated with free DOX, scND[DOX], or αND[DOX], and the level of fluorescent signal from the drug was measured at increasing time intervals by flow cytometry (FIG. 37C). For all samples, there was a time-dependent increase in drug uptake; delivery was the most effective for αND[DOX], with considerable differentiation observed compared with the other groups at 2 h and 4 h. This was corroborated when analyzing by fluorescence microscopy, where cells incubated with αND[DOX] exhibited the strongest signal after 2 h of incubation (FIG. 37D). The cytotoxicity of αND[DOX] after incubating with MDA-MB-231 cells (e.g., for 4 h) was further assessed, followed by replacement with fresh media and incubation for another 48 h (FIG. 37E). Compared to free DOX and scND[DOX], which exhibited IC₅₀ values of 0.81μg/mL and 0.80 μg/mL, respectively, αND[DOX] was more active with an IC₅₀ value of 0.25 μg/mL. The data confirmed that, with its enhanced affinity for EGFR, αND was able to better deliver incorporated payloads to improve activity against cancer cells overexpressing the tumor marker.

### Example In Vivo Characterization

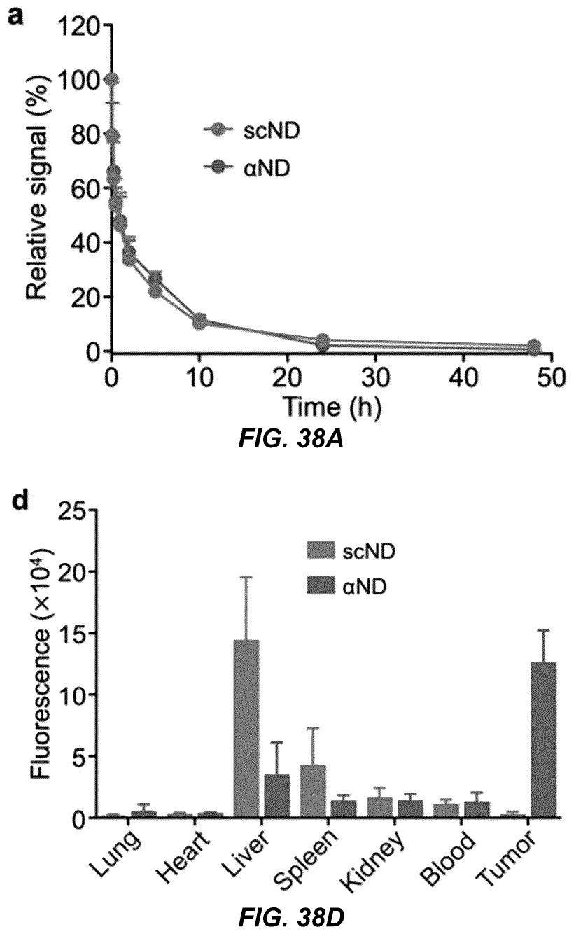

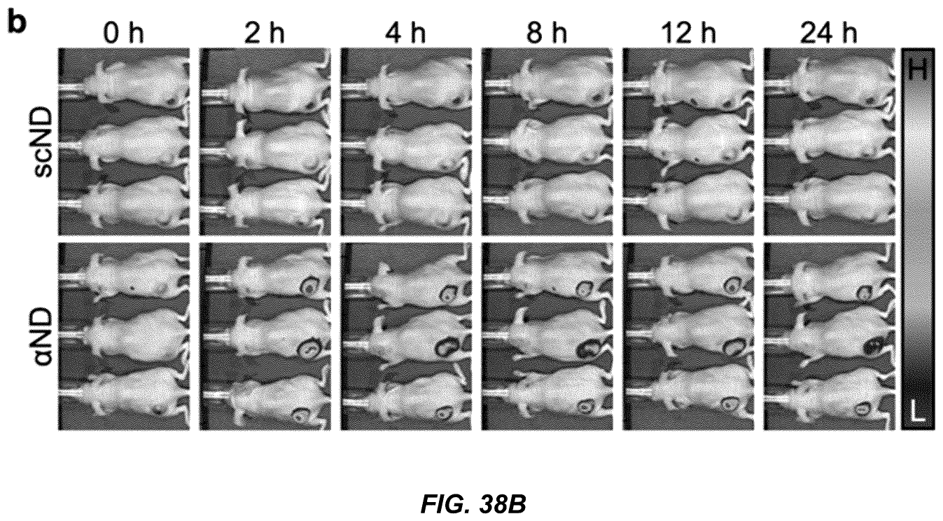

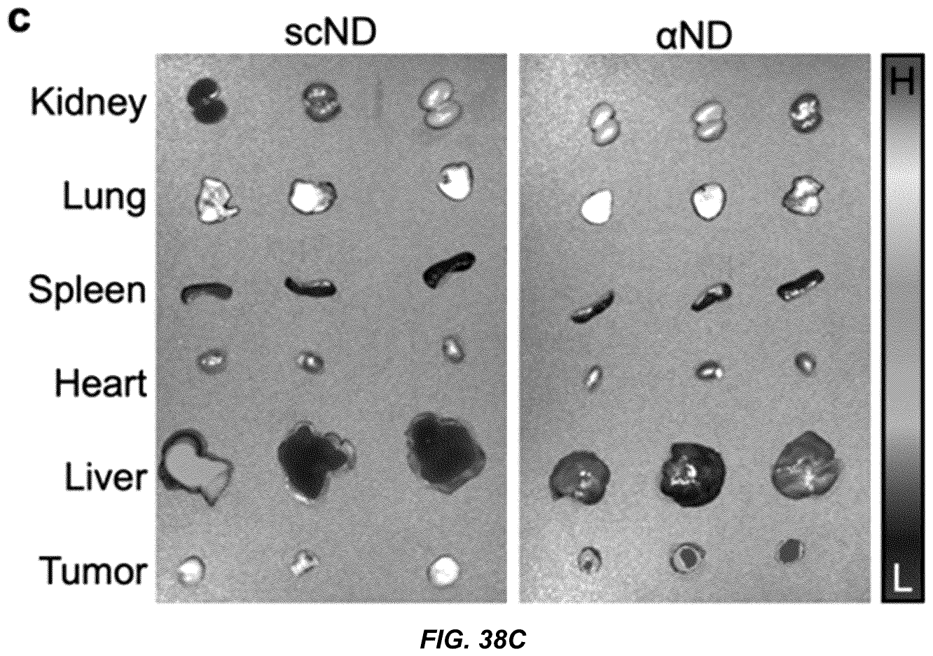

After in vitro characterization of the αND formulation, its in vivo performance was subsequently assessed. To evaluate pharmacokinetics, dye-labeled scND and αND were once again prepared. When administered intravenously, the pharmacokinetic profile of the αND formulation was near-identical its nonfunctionalized counterpart, indicating that the affibody conjugation did not have a pronounced effect on circulation (FIG. 38A).

FIGS. 38A-38C show data plots and data images depicting an example in vivo characterization of example embodiments of the CNDs. FIG. 38A shows a data plot depicting pharmacokinetic profiles of dye-labeled scND and αND after intravenous administration (n=3, mean+SD). FIG. 38B shows an image panel of live imaging of MDA-MB-231 tumor-bearing nu/nu mice after intravenous administration of dye-labeled scND and αND (H: high signal, L: low signal). FIG. 38C shows an image panel of ex vivo imaging of organs collected from MDA-MB-231 tumor-bearing nu/nu mice at 24 h after intravenous administration of dye-labeled scND and αND (H: high signal, L: low signal). FIG. 38D shows a data plot depicting total fluorescence of organ homogenates from MDA-MB-231 tumor-bearing nu/nu mice at 24 h after intravenous administration of dye-labeled scND and αND (n=3, mean+SD).

In vivo targeting was evaluated using a murine xenograft tumor model in which MDA-MB-231 cells were intradermally implanted into nude mice. The fluorescent signal of the nanoparticles was visualized over time using in vivo imaging, and significant localization of αNDs at the tumor site was observed as early as 2 h and up to 24 h after intravenous injection (FIG. 38B). In contrast, little to no tumor accumulation was observed for the scND formulation, confirming the exceptional targeting effect mediated by the affibody ligand. The tumor targeting was further confirmed at 24 h post injection, when the mice were euthanized, and their major organs were imaged ex vivo (FIG. 38C). While most of the signal was associated with the tumors for the αND group, the majority of the signal was found in the liver for scND. Finally, the organs and tumors were homogenized to measure their total fluorescent signal, which further supported the affinity of αND for the EGFR⁺ tumors (FIG. 38D).

### Example Antitumor Efficacy and in Vivo Safety

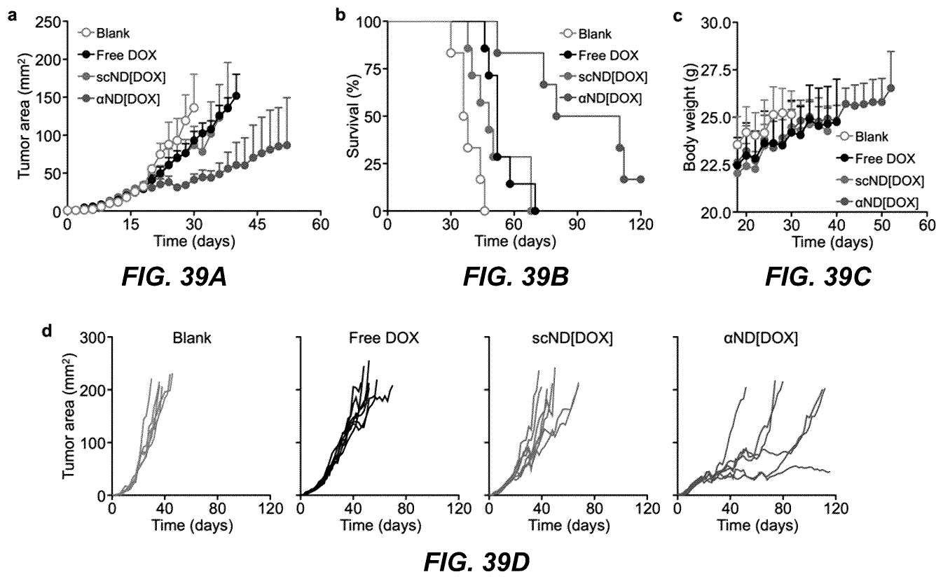

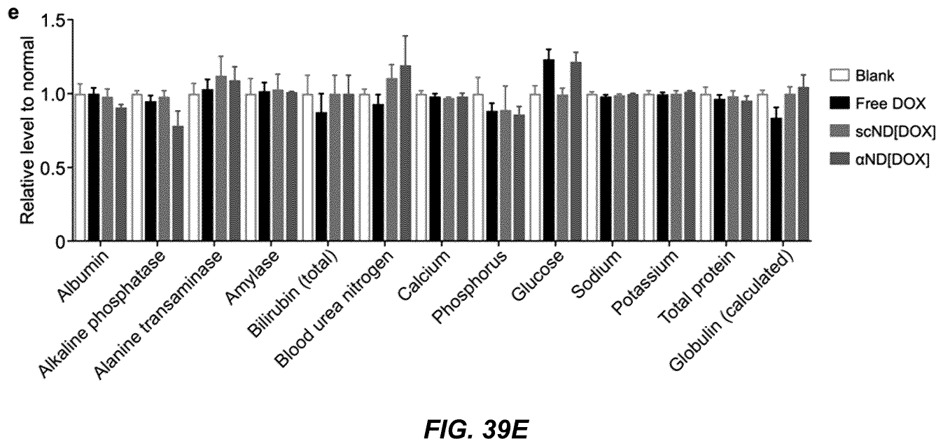

FIGS. 39A-39E show data plots depicting example results of example implementations to evaluate therapeutic efficacy and in vivo safety of example embodiments of CNDs. FIGS. 39A-39E show data plots of the average tumor size (FIG. 39A), survival (FIG. 39B), body weight (FIG. 39C), and individual tumor kinetics (FIG. 39D) of MDA-MB-231 tumor-bearing nu/nu mice treated with free DOX, scND[DOX], and αND[DOX] every 3 days for a total of 4 treatments after the tumor area reached 35 mm2 (n=6 for blank and αND[DOX], n=7 for free DOX and scND[DOX], mean+SD). FIG. 39E shows a data plot of serum biochemistry of immunocompetent mice at 24 h after receiving free DOX, scND[DOX], and αND[DOX].

As before, a xenograft tumor model was established by intradermal injection of MDA-MB-231 cells into nude mice. When the tumor area reached ˜35 mm², mice were treated intravenously with free DOX, scND[DOX], or αND[DOX] at a drug dosage of 2.5 mg/kg every three days for a total of four injections (FIG. 39A-39D). Compared to untreated mice, both free DOX and scND[DOX] had a moderate impact on tumor growth, prolonging median survival from 37 days to 52 and 48 days, respectively. Treatment with αND[DOX] further slowed the tumor growth kinetics and prolonged survival time to 95 days. In terms of safety, none of the treatments had a significant impact on body weight over the course of the study. Additionally, healthy and immunocompetent CD1 mice were treated with free DOX, scND[DOX], or αND[DOX] at a drug dosage of 2.5 mg/kg. Sera were collected 24 h later for comprehensive biochemistry analysis (FIG. 39E). Compared to untreated healthy mice, no major differences were observed among the tested parameters for all treatment groups, suggesting good biosafety at the drug dosage that was employed.

As demonstrated by the example implementations, for example, it is shown that the modularly functionalized cellular nanodisc platform in accordance with the present technology is capable of delivering chemotherapeutics more specifically to tumor sites. Source cells were modified to express SpyCatcher on their surface, which enabled CNDs fabricated from their plasma membrane to be covalently functionalized with a SpyTag-labeled affibody targeting EGFR. This enabled the resulting affibody-modified CND formulation to exhibit enhanced affinity to cancer cells overexpressing the tumor marker. It was further demonstrated that the nanodiscs could be loaded with a model chemotherapeutic, which improved the drug's cytotoxic activity in vitro against EGFR⁺ cells. In vivo, the targeted CNDs were better at localizing to tumors versus an untargeted control, allowing for better DOX delivery to inhibit tumor growth in a murine xenograft model. Overall, the example implementations validate the ability of CNDs to be rapidly modified using a modular functionalization approach, which can enable the platform to be generalized to many other targets.

### EXAMPLES

In some embodiments in accordance with the present technology (example A1), a modular functionalized nanoparticle includes a nanoparticle core; a cell membrane coating on the nanoparticle core; and a molecular binding complex natively bound to the cell membrane coating, the molecular binding complex comprising an anchor compound integrated with the cell membrane coating, and a tag molecule having a specific binding affinity to the anchor compound and bound to the anchor compound, wherein the tag molecule is configured to couple a functional substance to the molecular binding complex so as to facilitate one or more of targeting, attachment, or payload delivery by the modular functionalized nanoparticle on a target cell.

Example A2 includes the nanoparticle of example A1 or any of examples A1-A18, wherein the tag molecule is bound to the anchor compound through an iso-peptide covalent bond.

Example A3 includes the nanoparticle of example A1 or any of examples A1-A18, wherein the tag molecule is bound to the anchor compound through a coiled coil peptide interaction.

Example A4 includes the nanoparticle of example Al or any of examples A1-A18, wherein the specific binding affinity between the tag molecule and the anchor compound is exclusive by excluding off-target binding of other species to the anchor compound.

Example A5 includes the nanoparticle of example A1 or any of examples A1-A18, wherein the molecular binding complex includes a catcher/tag protein-ligand binding system or includes a coiled-coil peptide binding system.

Example A6 includes the nanoparticle of example A5 or any of examples A1-A18, wherein the molecular binding complex includes the catcher/tag protein-ligand binding system, and wherein the anchor compound includes SpyCatcher protein, and the tag molecule includes SpyTag peptide tag; or the anchor compound includes SnoopCatcher protein, and the tag molecule includes SnoopTag peptide tag; or the anchor compound includes DogCatcher protein, and the tag molecule includes DogTag peptide tag.

Example A7 includes the nanoparticle of example A5 or any of examples A1-A18, wherein the molecular binding complex includes the catcher/tag protein-ligand binding system, and wherein the molecular binding complex comprises a variety of different anchor compounds and a variety of different corresponding tag molecules, wherein the variety of different anchor compounds includes at least two of SpyCatcher protein, SnoopCatcher protein, or DogCatcher protein, and wherein the variety of different tag molecules includes at least two of SpyTag peptide tag, SnoopTag peptide tag, or DogTag peptide tag, respectively.

Example A8 includes the nanoparticle of example A5 or any of examples A1-A18, wherein the molecular binding complex includes the coiled-coil peptide binding system, wherein the anchor compound includes a protein with a leucine zipper motif, and the tag molecule includes a protein or peptide having a corresponding leucine zipper motif.

Example A9 includes the nanoparticle of example A1 or any of examples A1-A18, wherein the cell membrane coating is derived from a cell membrane of a cell genetically-engineered to express the anchor compound that is natively bound to the cell membrane.

Example A10 includes the nanoparticle of example A1 or any of examples A1-A18, wherein the functional substance includes a target ligand having a particular binding affinity to a receptor of the target cell.

Example A11 includes the nanoparticle of example A10 or any of examples A1-A18, wherein the functional substance includes a functional soluble ligand selected from a group consisting of a small molecule, a peptide, an aptamer, a protein, a nucleic acid, and a nucleotide, or derivative of any thereof.

Example A12 includes the nanoparticle of example A10 or any of examples A1-A18, further comprising a payload molecule attached to a portion of or incorporated within the modular functionalized nanoparticle, wherein the payload molecule is able to be uptaken by the target cell based on the attachment or uptake of the modular functionalized nanoparticle to or by the target cell.

Example A13 includes the nanoparticle of example A12 or any of examples A1-A18, wherein the payload molecule includes a drug, a prodrug, a nucleic acid substance, a protein substance, or a lipid substance.

Example A13a includes the nanoparticle of example A13 or any of examples A1-A18, wherein the nucleic acid substance includes one or more of a nucleotide, an oligonucleotide, an oligonucleotide-based aptamer, a deoxyribonucleic acid (DNA) or portion thereof, or a ribonucleic acid (RNA) or portion thereof.

Example A13b includes the nanoparticle of example A13 or any of examples A1-A18, wherein the protein substance includes one or more of an amino acid, a peptide, a peptide-based aptamer, an enzyme, an antibody, or a hormone.

Example A13c includes the nanoparticle of example A13 or any of examples A1-A18, wherein the lipid substance includes one or more of a liposome, a glyceride, a fatty acid, a steroid, or a phospholipid.

Example A14 includes the nanoparticle of example A1 or any of examples A1-A18, wherein the functional substance includes a payload substance to be uptaken by the target cell.

Example A15 includes the nanoparticle of example A14 or any of examples A1-A18, wherein the payload substance includes a drug, a prodrug, a nucleic acid substance, a protein substance, or a lipid substance.

Example A15a includes the nanoparticle of example A15 or any of examples A1-A18, wherein the nucleic acid substance includes one or more of a nucleotide, an oligonucleotide, an oligonucleotide-based aptamer, a deoxyribonucleic acid (DNA) or portion thereof, or a ribonucleic acid (RNA) or portion thereof.

Example A15b includes the nanoparticle of example A15 or any of examples A1-A18, wherein the protein substance includes one or more of an amino acid, a peptide, a peptide-based aptamer, an enzyme, an antibody, or a hormone.

Example A15c includes the nanoparticle of example A15 or any of examples A1-A18, wherein the lipid substance includes one or more of a liposome, a glyceride, a fatty acid, a steroid, or a phospholipid.

Example A16 includes the nanoparticle of example A1 or any of examples A1-A18, wherein the nanoparticle core includes a nanosphere, a nanodisc, a nanorod, or a nanocone.

Example A17 includes the nanoparticle of example A1 or any of examples A1-A18, wherein the nanoparticle core includes at least one of a polymeric nanoparticle, an inorganic nanoparticle, a ceramic nanoparticle, a carbon nanoparticle, a lipid nanoparticle, a protein nanoparticle, or a hydrogel nanoparticle.

Example A18 includes the nanoparticle of example A1 or any of examples A1-A17, wherein the nanoparticle core has at least one size dimension in a range of 30 nm to 1 μm.

In some embodiments in accordance with the present technology (example A19), a method for fabricating a modular functionalized nanoparticle includes forming a cell membrane coated nanoparticle, wherein the cell membrane coated nanoparticle comprises a nanoparticle core and a cell membrane coating on the nanoparticle core having an anchor compound natively bound to the cell membrane coating; forming a modular molecular linkage complex, wherein the modular molecular linkage complex comprises (i) a tag molecule having a specific binding affinity to the anchor compound, and (ii) a functional substance coupled to the tag molecule; and producing the modular functionalized nanoparticle by functionalizing the cell membrane coated nanoparticle with the modular molecular linkage complex.

Example A20 includes the method of example A19 or any of examples A19-A26, wherein the producing the modular functionalized nanoparticle comprises a spontaneous, exclusive, high binding affinity molecular interaction between the anchor compound and the tag molecule coupled to the functional substance.

Example A21 includes the method of example A19 or any of examples A19-A26, wherein the functional substance provides a binding affinity to a receptor of a target cell for enabling attachment or uptake of the modular functionalized nanoparticle to or by the target cell.

Example A22 includes the method of example A19 or any of examples A19-A26, wherein the forming the molecular linkage complex comprises conjugating a peptide tag as the tag molecule with a functional soluble ligand as the functional substance.

Example A23 includes the method of example A19 or any of examples A19-A26, further comprising attaching a payload to the modular functionalized nanoparticle.

Example A24 includes the method of example A19 or any of examples A19-A26, further comprising genetically-engineering a cell to express the anchor protein integrated within the cell membrane of the cell.

Example A25 includes the method of example A19 or any of examples A19-A26, further comprising selecting the functional substance based on a property of the functional substance that selectively targets, binds, or otherwise interacts with a receptor, lipid, or other unique molecular identifier of a target cell to enable attachment, interaction, or uptake of the modular functionalized nanoparticle to or by the target cell.

Example A26 includes the method of example A19 or any of examples A19-A25, wherein the produced modular functionalized nanoparticle includes the modular functionalized nanoparticle of any of examples A1-A18.

In some embodiments in accordance with the present technology (example B1), a modular functionalized nanoparticle includes a nanoparticle core; a cell membrane coating on the nanoparticle core; a molecular binding complex natively bound to the cell membrane coating, the molecular binding complex comprising an anchor protein integrated with a lipid bilayer of the cell membrane coating, and a peptide tag having a binding affinity to an exposed region of the anchor protein from the cell membrane coating; and a target ligand coupled to the peptide tag of the molecular binding complex, wherein the target ligand has a binding affinity to a receptor of a target cell for enabling attachment or uptake of the modular functionalized nanoparticle to or by the target cell.

Example B2 includes the nanoparticle of example B1, wherein the modular functionalized nanoparticle includes one or more features of the modular functionalized nanoparticle of any of examples A1-A18.

In some embodiments in accordance with the present technology (example C1), a modular functionalized nanoparticle includes a nanoparticle core; a cell membrane coating on the nanoparticle core; an anchor protein natively bound to the cell membrane coating; and a modular linkage complex conjugated to the anchor protein, the modular linkage complex comprising a peptide tag having a binding affinity to a region of the anchor protein exposed from the cell membrane coating, and a target ligand coupled to the peptide tag, wherein the target ligand has a binding affinity to a receptor of a target cell for enabling attachment or uptake of the modular functionalized nanoparticle to or by the target cell.

Example C2 includes the nanoparticle of example C1, wherein the modular functionalized nanoparticle includes one or more features of the modular functionalized nanoparticle of any of examples A1-A18.

In some embodiments in accordance with the present technology (example D1), a modular functionalized nanoparticle includes a nanoparticle core; a cell membrane coating on the nanoparticle core; an anchor protein natively bound to the cell membrane coating; a peptide tag having a binding affinity to a region of the anchor protein exposed from the cell membrane coating; and a target ligand coupled to the peptide tag, wherein the target ligand has a binding affinity to a receptor of a target cell for enabling attachment or uptake of the modular functionalized nanoparticle to or by the target cell.

Example D2 includes the nanoparticle of example D1, wherein the modular functionalized nanoparticle includes one or more features of the modular functionalized nanoparticle of any of examples A1-A18.

### CONCLUSION

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A modular functionalized nanoparticle, comprising:
a nanoparticle core;
a cell membrane coating on the nanoparticle core; and
a molecular binding complex natively bound to the cell membrane coating, the molecular binding complex comprising an anchor compound integrated with the cell membrane coating, and a tag molecule having a specific binding affinity to the anchor compound and bound to the anchor compound,
wherein the tag molecule is configured to couple a functional substance to the molecular binding complex so as to facilitate one or more of targeting, attachment, or payload delivery by the modular functionalized nanoparticle on a target cell.

2. The nanoparticle of claim 1, wherein the tag molecule is bound to the anchor compound through an iso-peptide covalent bond.

3. The nanoparticle of claim 1, wherein the tag molecule is bound to the anchor compound through a coiled coil peptide interaction.

4. The nanoparticle of claim 1, wherein the specific binding affinity between the tag molecule and the anchor compound is exclusive by excluding off-target binding of other species to the anchor compound.

5. The nanoparticle of claim 1, wherein the molecular binding complex includes a catcher/tag protein-ligand binding system or includes a coiled-coil peptide binding system.

6. The nanoparticle of claim 5, wherein the molecular binding complex includes the catcher/tag protein-ligand binding system, and wherein
the anchor compound includes SpyCatcher protein, and the tag molecule includes SpyTag peptide tag; or
the anchor compound includes SnoopCatcher protein, and the tag molecule includes SnoopTag peptide tag; or
the anchor compound includes DogCatcher protein, and the tag molecule includes DogTag peptide tag.

7. The nanoparticle of claim 5, wherein the molecular binding complex includes the catcher/tag protein-ligand binding system, and wherein the catcher/tag protein-ligand binding system comprises a variety of different anchor compounds and a variety of different corresponding tag molecules, wherein the variety of different anchor compounds includes at least two of SpyCatcher protein, SnoopCatcher protein, or DogCatcher protein, and wherein the variety of different tag molecules includes at least two of SpyTag peptide tag, SnoopTag peptide tag, or DogTag peptide tag, respectively.

8. The nanoparticle of claim 5, wherein the molecular binding complex includes the coiled-coil peptide binding system, wherein the anchor compound includes a protein with a leucine zipper motif, and the tag molecule includes a protein or peptide having a corresponding leucine zipper motif.

9. The nanoparticle of claim 1, wherein the cell membrane coating is derived from a cell membrane of a cell genetically-engineered to express the anchor compound that is natively bound to the cell membrane.

10. The nanoparticle of claim 1, wherein the functional substance includes a target ligand having a particular binding affinity to a receptor of the target cell.

11. The nanoparticle of claim 10, wherein the functional substance includes a functional soluble ligand selected from a group consisting of a small molecule, a peptide, an aptamer, a protein, a nucleic acid, and a nucleotide.

12. The nanoparticle of claim 10, further comprising:
a payload molecule attached to a portion of or incorporated within the modular functionalized nanoparticle, wherein the payload molecule is able to be uptaken by the target cell based on the attachment or uptake of the modular functionalized nanoparticle to or by the target cell.

13. The nanoparticle of claim 12, wherein the payload molecule includes a drug, a prodrug, a nucleic acid substance, a protein substance, or a lipid substance.

14. The nanoparticle of claim 1, wherein the functional substance includes a payload substance to be uptaken by the target cell.

15. The nanoparticle of claim 14, wherein the payload substance includes a drug, a prodrug, a nucleic acid substance, a protein substance, or a lipid substance.

16. The nanoparticle of claim 1, wherein the nanoparticle core includes a nanosphere, a nanodisc, a nanorod, or a nanocone.

17. The nanoparticle of claim 1, wherein the nanoparticle core includes at least one of a polymeric nanoparticle, an inorganic nanoparticle, a ceramic nanoparticle, a carbon nanoparticle, a lipid nanoparticle, a protein nanoparticle, or a hydrogel nanoparticle.

18. The nanoparticle of claim 1, wherein the nanoparticle core has at least one size dimension in a range of 30 nm to 1 μm.

19. A method for fabricating a modular functionalized nanoparticle, the method comprising:
forming a cell membrane coated nanoparticle, wherein the cell membrane coated nanoparticle comprises a nanoparticle core and a cell membrane coating on the nanoparticle core having an anchor compound natively bound to the cell membrane coating;
forming a modular molecular linkage complex, wherein the modular molecular linkage complex comprises (i) a tag molecule having a specific binding affinity to the anchor compound, and (ii) a functional substance coupled to the tag molecule; and
producing the modular functionalized nanoparticle by functionalizing the cell membrane coated nanoparticle with the modular molecular linkage complex.

20. The method of claim 19, wherein the producing the modular functionalized nanoparticle comprises a spontaneous, exclusive, high binding affinity molecular interaction between the anchor compound and the tag molecule coupled to the functional substance.

21. The method of claim 19, wherein the functional substance provides a binding affinity to a receptor of a target cell for enabling attachment or uptake of the modular functionalized nanoparticle to or by the target cell.

22. The method of claim 19, wherein the forming the molecular linkage complex comprises conjugating a peptide tag as the tag molecule with a functional soluble ligand as the functional substance.

23. The method of claim 19, further comprising:
attaching a payload to the modular functionalized nanoparticle.

24. The method of claim 19, further comprising:
genetically-engineering a cell to express the anchor protein integrated within the cell membrane of the cell.

25. The method of claim 19, further comprising:
selecting the functional substance based on a property of the functional substance that selectively targets, binds, or otherwise interacts with a receptor, lipid, or other unique molecular identifier of a target cell to enable attachment, interaction, or uptake of the modular functionalized nanoparticle to or by the target cell.
